(19) United States
(12) Patent Application Publication
(10) Pub. No.: US 2018/0030080 A1
(43) Pub. Date: Feb. 1, 2018

(54) Phosphoramidate nucleoside prodrug for treating viral diseases and cancer, processes for their preparation and their use

(71) Applicants: Alexandre Vasilievich Ivachtchenko, Hallandale, FL (US); Andrey Alexandrovich Ivashchenko, Moscow (RU); Nikolay Filippovich Savchuk, Rancho Santa Fe, CA (US); Alena Alexandrovna Ivachtchenko, Hallandale, FL (US); Oleg Dmitrievich Mitkin, Khimki (RU); ASAVI, LLC, Hallandale Beach, FL (US)

(72) Inventors: Alexandre Vasilievich Ivachtchenko, Hallandale, FL (US); Oleg Dmitrievich Mitkin, Khimki (RU); Andrey Alexandrovich Ivashchenko, Moscow (RU); Alena Alexandrovna Ivachtchenko, Hallandale, FL (US); Nikolay Filippovich Savchuk, Rancho Santa Fe, CA (US)

(21) Appl. No.: 15/221,613

(22) Filed: Jul. 28, 2016

(51) Int. Cl. C07H 19/16; C07H 19/06; C07F 9/6558; C07F 9/6561

(52) U.S. Cl. CPC C07H 19/16; C07F 9/65616; C07H 19/06; C07F 9/65586

(57) ABSTRACT

The present invention pertains to chemotherapeutic agents and their use for treating viral and cancerous diseases. These compounds are inhibitors of HCV NS5B polymerase, HBV DNA polymerase and, HIV-1 reverse transcriptase (RT) inhibitor, and for treatment of hepatitis B and C infection in mammals. These compounds are also of interest for the treatment of cancer.

The phosphoramidate nucleoside prodrug of the general formula 1, a stereoisomer, isotope-enriched analogue, pharmaceutically acceptable salt, hydrate, solvate, or crystalline or polymorphic form thereof,

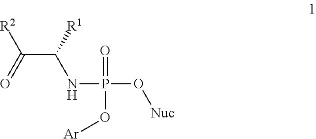

wherein: Ar is aryl or hetaryl; R¹ is H or CH₃; R² is the substituent selected from OCH₂CH═CH₂, OCH₂CH≡CH, OCH₂CH₂CH₂OCH₃,

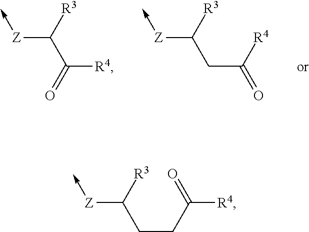

R³ is H or CH₃; R⁴ is OH, OR⁵, NR⁶R⁷; R⁵ is C₁-C₄-alkyl; R⁶ and R⁷ are not necessarily the same substituents selected from H or CH₃; Z═O, or NH; an arrow (→) indicates the place of substituent connection;
Nuc is

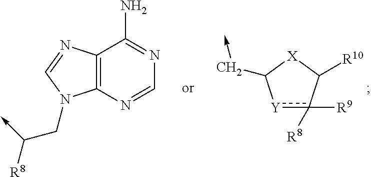

R⁸ and R⁹ are not necessarily the same substituents selected from H, F, Cl, CH₃ or OH provided when continuous line and its accompanying dotted line ( [symbol] ) together are the single carbon-carbon (C—C) bond or R⁸ and R⁹ are hydrogen provided when continuous line and its accompanying dotted line ( [symbol] ) together are the double carbon-carbon bond (C═C);
R¹⁰ is the substituent selected from R^{10.1}-R^{10.5};

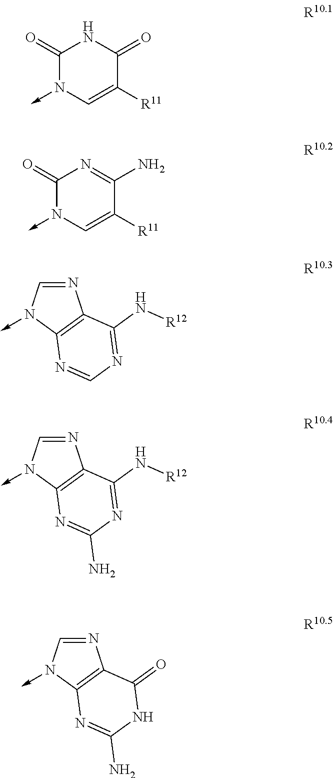

R¹¹ is the substituent selected from H, F, Cl, CH₃, or CF₃;
R¹² is hydrogen, C₁-C₄-alkyl or C₃-C₆-cycloalkyl;
X is oxygen or ethanediyl-1,1 (C═CH₂);
Y is O, S, CH₂, or HO—CH group provided when continuous line and its accompanying dotted line ( [symbol] ) together are the single carbon-carbon (C—C) bond or Y is CH group provided when continuous line and its accompanying dotted line ( [symbol] ) together are the double carbon-carbon bond (C═C), and compound of the general formula 1, stereoisomers, isotope-enriched analogues, pharmaceutically acceptable salts, hydrates, solvates, or crystalline or polymorphic forms thereof, wherein:
Ar is aryl or hetaryl;
R¹ is H or CH₃;
R is isopropyl;
Nuc is

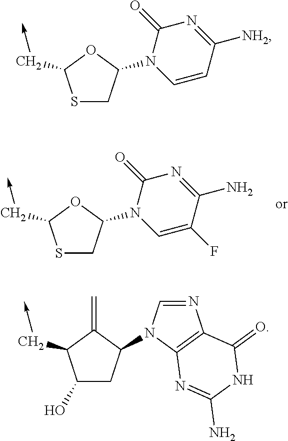

### FIELD OF INVENTION

The present invention pertains to chemotherapeutic agents and their use for treating viral and cancerous diseases. These compounds are inhibitors of HCV NS5B polymerase, HBV DNA polymerase and, HIV-1 reverse transcriptase (RT) inhibitor, and for treatment of hepatitis B and C infection in mammals. These compounds are also of interest for the treatment of cancer.

### BACKGROUND

Nucleosides and nucleotides (nucleos(t)ides) have been in clinical use for almost 50 years and have become cornerstones of treatment for patients with viral infections or cancer. The approval of several additional drugs over the past decade demonstrates that this family still possesses strong potential. Therefore nucleosides (Nuc) and their analogs 2′-deoxy-L-uridine (Nuc1), 2′-deoxy-D-uridine (Nuc2), telbivudine (Nuc3), zidovudine (Nuc4), trifluridine (Nuc5), clevudine (Nuc6), PSI-6206 (Nuc7), 2′-(S)-2′-chloro-2′-deoxy-2′-fluorouridine (Nuc8), ND06954 (Nuc9), stavudine (Nuc10), festinavir (Nuc11), torcitabine (Nuc12), (−)-beta-D-(2R,4R)-dioxolane-thymine (Nuc13), 2-(6-amino-purin-9-yl)-ethanol (Nuc14), (R)-1-(6-amino-9H-purin-9-yl)propan-2-ol (Nuc15), 2′-C-methylcytidine (Nuc16), PSI-6130 (Nuc17), gemcitabine (Nuc18), 2′-chloro-2′-deoxy-2′-fluorocytidine (Nuc19), 2′,2′-dihloro-2′-deoxycytidine (Nuc20), lamivudine (Nuc21), emtricitabine (Nuc22), 2′-deoxyadenosine (Nuc23), 2′-deoxy-β-L-adenosine (Nuc24), 2′-deoxy-4′-C-ethynyl-2-fluoroadenosine (Nuc25), [(2R,4R)-4-(6-cyclopropylamino-purin-9-yl)-[1,3]dioxolan-2-yl]-methanol (Nuc26), amdoxovir (Nuc27), entecavir (Nuc28), FMCA (Nuc29), dioxolane-G (Nuc30), β-D-2′-deoxy-2′-(R)-fluoro-2′-β-C-methylguanosine (Nuc31), abacavir (Nuc32), didanosine (Nuc33), and others are of great interest as promising chemotherapeutic agents [M. J. Sofia. Nucleosides and Nucleotides for the treatment of viral diseases. In Annual Reports in Medicinal Chemistry 2014, Volume 49, Editor-in-Chief M. C. Desai, p 221-247. L. P. Jordheim et al. Advances in the development of nucleoside and nucleotide analogues for cancer and viral diseases. Nat. Rev. Drug. Discov. 2013 June; 12(6), 447-464.].

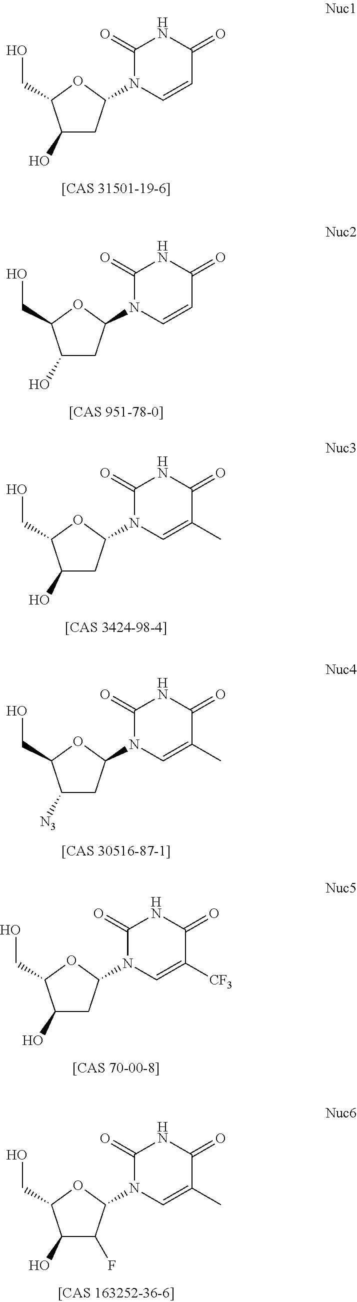 [SMILES: O=c1ccn([C@@H]2C[C@@H](O)[C@H](CO)O2)c(=O)[nH]1 ; O=c1ccn([C@H]2C[C@H](O)[C@@H](CO)O2)c(=O)[nH]1 ; Cc1cn([C@@H]2C[C@@H](O)[C@H](CO)O2)c(=O)[nH]c1=O ; Cc1cn([C@H]2C[C@H](N=[N+]=[N-])[C@@H](CO)O2)c(=O)[nH]c1=O ; O=c1[nH]c(=O)n([C@@H]2C[C@@H](O)[C@H](CO)O2)cc1C(F)(F)F ; Cc1cn([C@H]2O[C@@H](CO)[C@H](O)C2F)c(=O)[nH]c1=O]

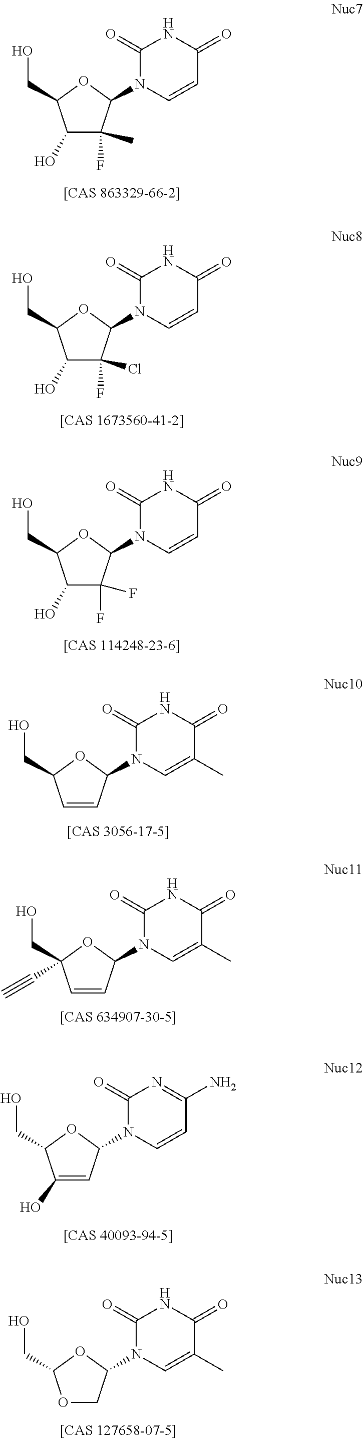

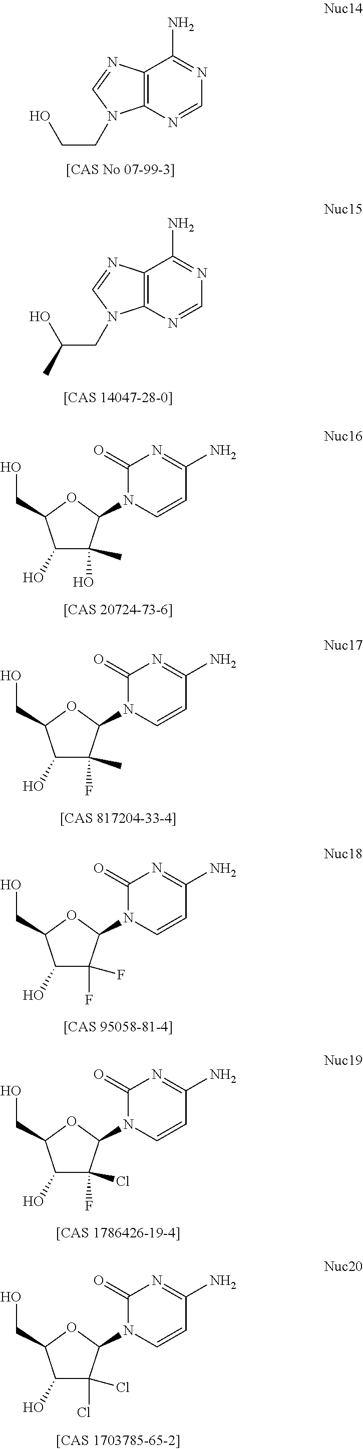

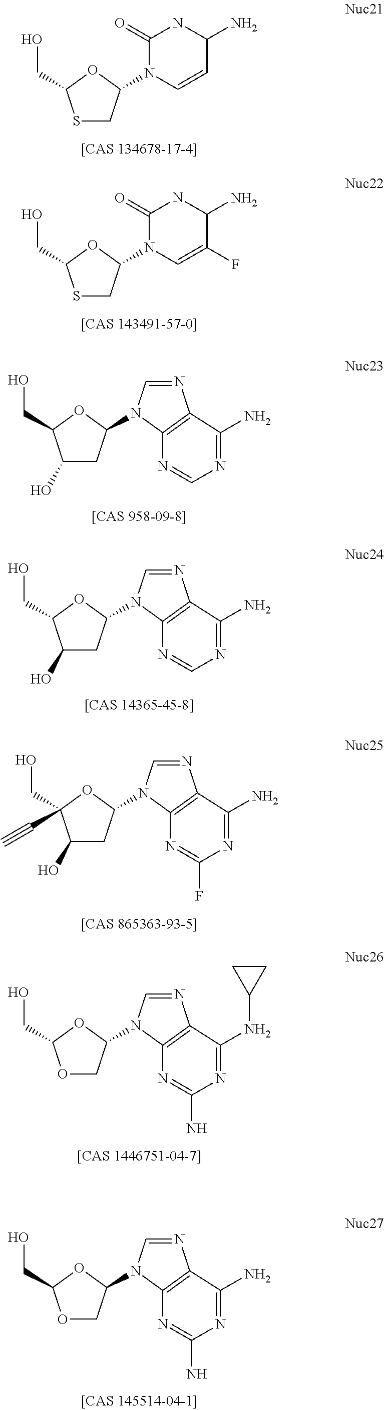

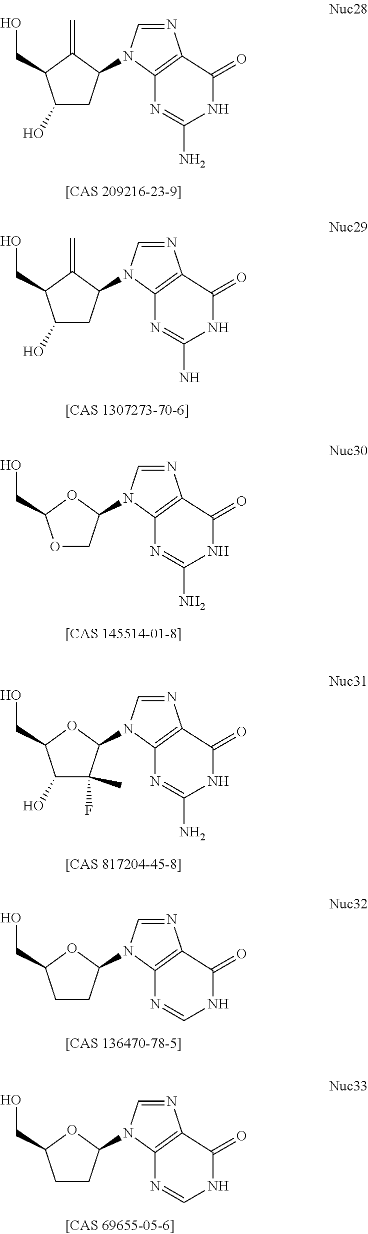 [SMILES: C=C1[C@H](CO)[C@@H](O)C[C@@H]1n1cnc2c(=O)[nH]c(N)nc21 ; C=C1[C@H](CO)[C@@H](O)C[C@@H]1n1cnc2c(=O)[nH]c(N)nc21 ; Nc1nc2c(ncn2[C@H]2CO[C@@H](CO)O2)c(=O)[nH]1 ; C[C@@]1(F)[C@H](O)[C@@H](CO)O[C@H]1n1cnc2c(=O)[nH]c(N)nc21 ; O=c1[nH]cnc2c1ncn2[C@H]1CC[C@@H](CO)O1 ; O=c1[nH]cnc2c1ncn2[C@H]1CC[C@@H](CO)O1]

Nucleos(t)ides have played an integral role in the treatment of viral diseases. For patients with human immunodeficiency virus (HIV) they have proven to be the backbone in number of combination regimens. Currently, nucleos(t)ides are the preferred option and standard of care for treating patients infected with hepatitis B virus (HBV) and they are emerging as a key component in therapies to treat hepatitis C virus (HCV) infection. They also play a central role in the management of other viral infections such as those caused by herpes viruses (HSV-1 and HSV-2), varicella zoster virus, Epstein-Barr virus, and cytomegalovirus [E. De Clercg. Ed. Antiviral Agents 2013, Vol. 67: Academic Press: New York. 2013. L. P. Jordheim et al. Advances in the development of nucleoside and nucleotide analogues for cancer and viral diseases. Nal. Rev. 2013, 12, 447-464.]. The attractiveness of a nucleos(t)ides strategy in the development of therapeutics for vital diseases caused the fact that all viruses require a polymerase for either DNA or RNA replication.

Another factor that must be considered when developing a nucleos(t)ide inhibitor pertains to nucleos(t)ide metabolic activation. It is the nucleotide triphosphate analog, as the functional substrate for the viral polymerase, that becomes incorporated into the growing RNA or DNA chain, typically leading to a chain termination event and ultimately an end to viral replication. Consequently, the efficiency by which a nucleos(t)ide gets converted to the active triphosphate and the concentration and half-life of the triphosphate within the cell are important factors in how effective the nucleos(t)ide is as an inhibitor of viral replication. In general, the first phosphorylation step is the most discriminating among the three needed to generate the active triphosphate. In cases where the nucleoside itself is not a good substrate for the kinase involved in the initial phosphorylation step, delivery of the monophosphate is desired, but this typically requires the use of prodrug technology to mask the unfavorable characteristics of the phosphate group and facilitate permeability. Consequently, nucleotide prodrug strategics have seen much use in the development of nucleotides to treat viral and cancer diseases.

HIV is a retrovirus that infects approximately 35 million individuals worldwide. HIV requires a RNA-dependent DNA polymerase or reverse transcriptase (RT) for replication of the viral genre. A number of nucleos(t)ide RT inhibitors have been approved for the treatment of HIV infection [R. F. Shinazi et al. Pharmacology of current and promising nucleosides for the treatment of human immunodeficiency viruses. J. Antiviral Res. 2006, 71, 322-334. E. De Clereq. he nucleoside reverse transcriptase inhibitors, nonnucleoside reverse transcriptase inhibitors, and protease inhibitors in the treatment of HIV infections (AIDS). Adva Pharmacol. 2013, 67, 317-358]. Several have been co-formulated into fixed-dose combinations and in some cases co formulated with other HIV replication inhibitors affording convenient therapeutic regimens that have become the standard in highly active antiretroviral therapy (HAART). These fixed-dose combinations include Combivir®, Trizivir®, Epzicom®, Truvadas, Atriple, Stribile, and Compleras. Tnwade, Atriple, Complete, and Stribile include the nucleoside emtricitabine and the acyclic nucleotide tenofovir diisoproxil fumarate (TDF) while Comnbivir™, Trizivie, and Epzicoms comprise a two- or three-drug combination comprising the nucleosides zidovudine (AZT), lamivudine (3 TC), and/or abacavir (ABC) [R. F. Shinazi et al. Pharmacology of current and promising nucleosides for the treatment of human immunodeficiency viruses. J. Antiviral Res. 2006, 71, 322-334.]. The success of HAART has made HIV a manageable disease and has dramatically increased the life expectancy of those infected with this once terminal illness. However, even with this success the search continues for new agents that address the needs of a chronically infected population where resistance, side effects due to long-term use, and drug-drug interactions are increasingly prevalent especially within an aging HIV population.

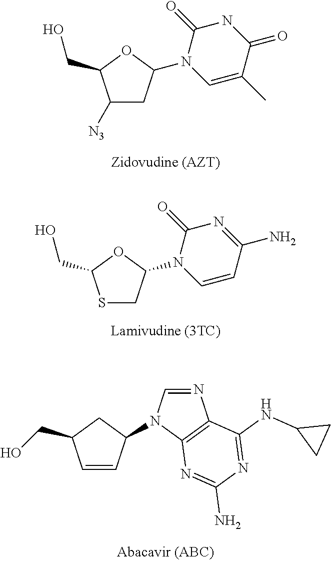 [SMILES: Cc1cn(C2CC(N=[N+]=[N-])[C@@H](CO)O2)c(=O)[nH]c1=O ; Nc1ccn([C@@H]2CS[C@H](CO)O2)c(=O)n1 ; Nc1nc(NC2CC2)c2ncn([C@H]3C=C[C@@H](CO)C3)c2n1]

In an effort to increase targeted exposure of tenofovir (TFV) in peripheral blood mononuclear cells (PBMCs) and consequently reduce the renal toxicity associated with TDF, a phosphoramidate prodrug GS-7340 ((S)-Isopropyl 2-(((S)-((((R)-1-(6-amino-9H-purin-9-yl)propan-2-yl)oxy)methyl)(phenoxy)-phosphoryl)amino)propanoate, tenofovir alafenamide, TAF), was developed. TAF was determined to be 400-fold more potent than TFV (1) in PBMCs and cleavage to TFV was mediated by lysosomal cathepsin A which is highly expressed in these cells. The use of this prodrug approach resulted in an enhanced exposure ratio of the parent nucleoside TFV in P13MCs relative to plasma and led to higher efficacy.

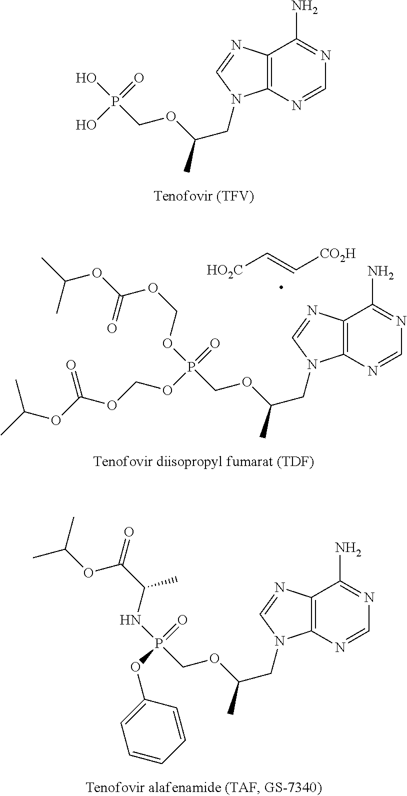

A search for a nucleotide phosphonate that would provide an improved resistance profile over existing nucleos(t)ides and that would exhibit a better safety profile relative to host DNA polymerases led to the 2′-F-2′,3¹-dideoxydidehydro-adenosine derivative GS-9148. This phosphonate nucleoside showed an improved resistance profile across a wide range of resistance mutations relative to all nucleos(t)ides in clinical use. In order to improve cell permeability properties and uptake into lymphoid cells, phosphoramidate prodrugs were evaluated as was previously demonstrated in the case of GS-9340. Ultimately, the in vivo profile and PBMC loading characteristics led to the identification of GS-9131 as a lead development candidate. In vitro and in vivo studies showed that GS-9131 had a reduced potential for renal accumulation relative to TDF and no significant renal findings were observed in 28-day toxicity studies in multiple species [M. J. Sofia. Nucleosides and Nucleotides for the treatment of viral diseases. In Annual Reports in Medicinal Chemistry 2014, Volume 49, Editor-in-Chief M. C. Desai, p 224.].

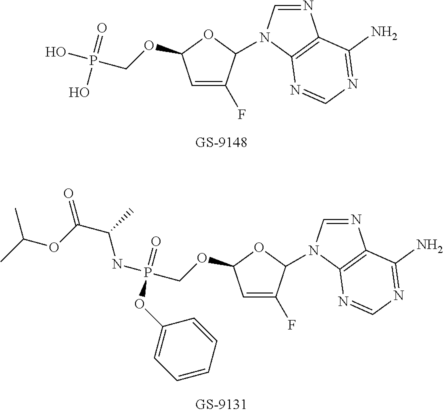 [SMILES: Nc1ncnc2c1ncn2C1O[C@H](OCP(=O)(O)O)C=C1F ; CC(C)OC(=O)[C@H](C)N[P@](=O)(CO[C@@H]1C=C(F)C(n2cnc3c(N)ncnc32)O1)Oc1ccccc1]

(−)-13-D-(2R,4R)-Dioxolane-thyznine (DOT, 23) is a dioxolane nucleoside thymidine mimetic that was shown in vitro to be active against both wild-type (EC₅₀=6.5 μM) and clinically significant nucleoside-resistant HIV-1 subtypes. However, DOT is a poor substrate in the first step of the phosphorylation cascade to the active triphosphate. An extensive SAR study was undertaken to assess phosphoramidate prodrugs identifying several such as D-dioxolane-thymine 5′-(4-bromophenyl ethyloxy-L-alanyl-phosphate) (DOT-phosphoramidate, ECₛₒ=0.23 pM) having submicromolar activity. The last in 75-fold more potency relative to DOT. Antiviral efficacy of the DOT phosphoramidates was far superior to that of the parent nucleoside. It has been shown that by phosphoramidate of DOT L-alanine is the preferred amino acid unit, that small alkyl, cycloalkyl or benzyl esters provide maximal potency and that simple phenyl phosphate esters are desired. [P. Wang et al. Phosphoramidate prodrugs of (−)-β-D-(2R,4R)-dioxolane-thymine (DOT) as potent anti-HIV agents. Antiviral Chem. Chemotherapy 2012, 22, 217-238].

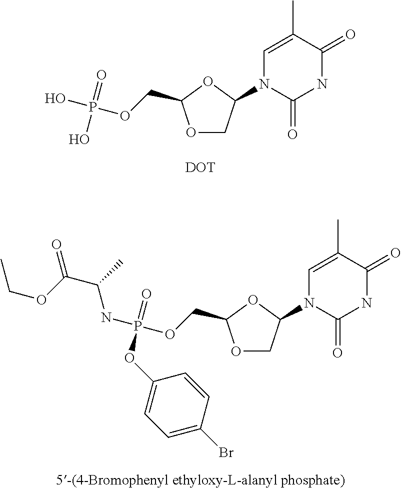

Similarly, phosphoramidate prodrugs of 6-substituted-2-H-purine dioxolanes were investigated as double prodrugs that would afford dioxolane-A mono-phosphate. The most potent and least cytotoxic analog was dioxolane-A mono-phosphoramidate EC₅₀=0.086 μM) [L. Bondada et al. Adenosine Dioxolane Nucleoside Phosphoramidates as Antiviral Agents for Human Immunodeficiency and Hepatitis B Viruses. ACS Med. Chem. Lett. 2013, 4, 747-751].

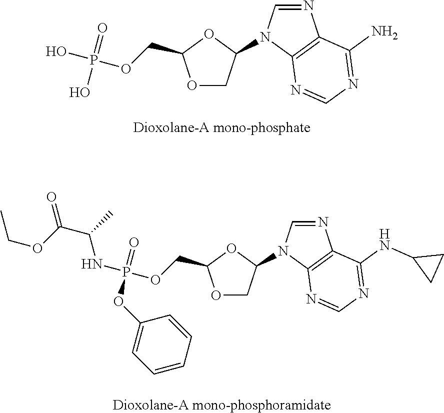 [SMILES: Nc1ncnc2c1ncn2[C@H]1CO[C@@H](COP(=O)(O)O)O1 ; "Dioxolane-A mono-phosphate" ; CCOC(=O)[C@H](C)N[P@](=O)(OC[C@@H]1OC[C@H](n2cnc3c(NC4CC4)ncnc32)O1)Oc1ccccc1 ; "Dioxolane-A mono-phosphoramidate"]

Recently it was shown that a phosphoramidate prodrug of 2′-deoxy-2′-α-fluoro-β-C-methyluridine-5′-monophosphate PSI-7851, demonstrates potent anti-hepatitis C virus (HCV) activity both in vitro and in vivo [E. Murakami et al. Mechanism of activation of PSI-7851 and its diastereoisomer PSI-7977. J. Biol. Chem. 2010, 285(45), 34337-34347]. PSI-7851 is a mixture of two diastereoisomers, PSI-7976 and PSI-7977, with PSI-7977 being the more active inhibitor of HCV RNA replication in the HCV replicon assay. To inhibit the HCV NS5B RNA-dependent RNA polymerase, PSI-7851 must be metabolized to the active triphosphate form.

The first step, hydrolysis of the carboxyl ester by human cathepsin A (CatA) and/or carboxylesterase 1 (CES1), is a stereospecific reaction. Hydrolysis of the ester is followed by a putative nucleophilic attack on the phosphorus by the carboxyl group resulting in the spontaneous elimination of phenol and the production of an alaninyl phosphate metabolite, PSI-352707, which is common to both isomers. The removal of the amino acid moiety of PSI-3 52707 is catalyzed by histidine triad nucleotide-binding protein 1 (Hint1) to give the 5′-monophosphate form PSI-7411. PSI-7411 is then consecutively phosphorylated to the diphosphate, PSI-7410, and to the active triphosphate metabolite, PSI-7409, by UMP-CMP kinase and nucleoside diphosphate kinase, respectively.

The scheme 1(a) of metabolism of PSI-7851 and its phosphoric diastereoisomers PSI-7976 and PSI-7977[E. Murakami et al. Mechanism of activation of PSI-7851 and its diastereoisomer PSI-7977. J. Biol. Chem. 2010, 285(45), 34337-34347].

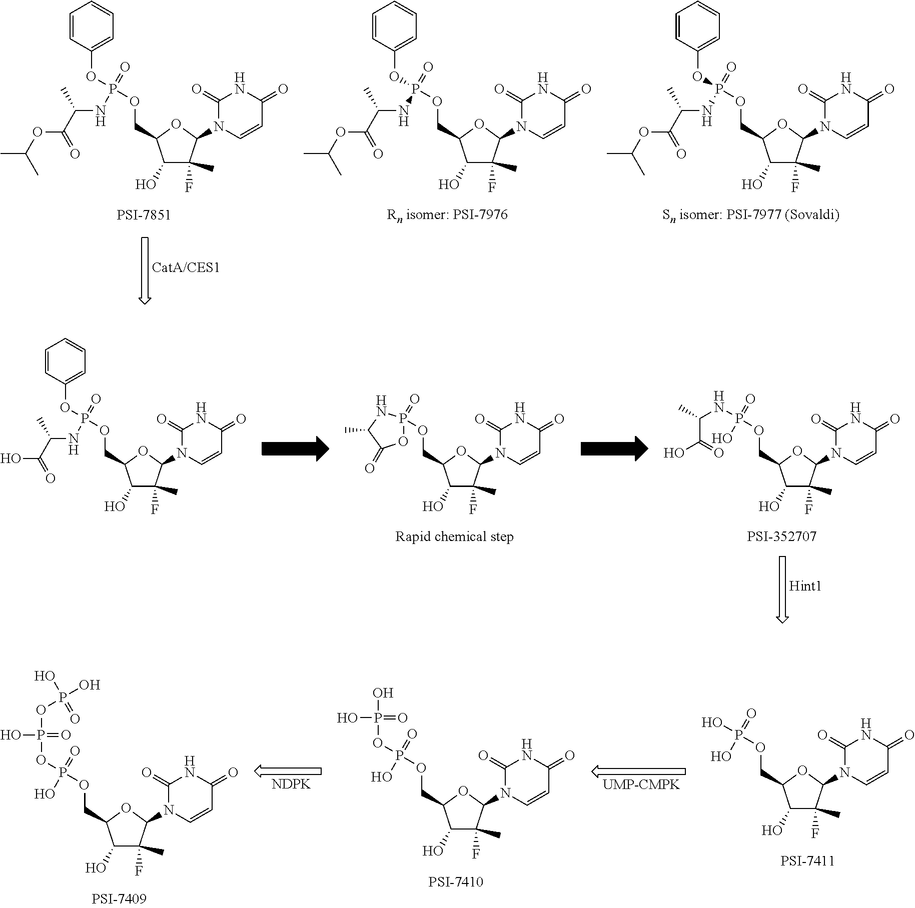

The uridine nucleotide prodrug PSI-7977 (sofosbuvir, Sovaldi®) [M. J. Sofia et al. Discovery of a β-D-20-Deoxy-20-r-fluoro-20-β-C-methyluridine Nucleotide Prodrug (PSI-7977) for the Treatment of Hepatitis C Virus. J. Med. Chem. 2010, 53, 7202-7218. M. J. Sofia et al. Nucleoside phosphoramidate prodrugs. U.S. Pat. No. 7,964,580 (2011).] became the first nucleos(t)ide approved by both the FDA and EU regulatory authorities for the treatment of HCV patients infected with genotype (gT) 1, 2, 3, and 4 HCV virus and in clinical trials it also showed efficacy against all relevant HCV gTs (1-6) [I. M. Jacobson et al. Sofosbuvir for hepatitis C genotype 2 or 3 in patients without treatment options. Engl. J. Med. 2013, 368, 1867-1877. E. Lewirz et al. Sofosbuvir for previously untreated chronic hepatitis C infection. Engl. J. Med. 2013, 368, 1878-1887]. Its approval marked the first introduction of an all-oral interferon (IFN)-free regimen to treat patients suffering from HCV infection.

Are also other known chemotherapeutic agents include phosphoramidate and nucleoside moieties to treat hepatitis C, including AVI-4201 [A. V. Ivachtchenko et al. Alkyl 2-{[(2r,3s,5r)-5-(4-amino-2-oxo-2H-pyrimidin-1-yl)-3-hydroxy-tetrahydro-furan-2-yl-methoxy]-phenoxy-phosphoryl-amino}-propionates, nucleoside inhibitors of HCV NS5B RNA-polymerase, and methods for producing and use thereof. WO2014148949, 2014], AVI-4203 [A. V. Ivachtchenko et al. Substituted (S)-(2R,3R,5R)-3-hydroxy-(5-pyrimidin-1-yl)-tetrahydrofuran-2-ylmethyl aryl phosphoramidate. U.S. Pat. No. 8,889,701, 2014] or CC-1845 [D. L Mayers. Development of Potent Novel Oral Pan-genotypic HCV Nucleotide, NS5A, NS5B non-nucleoside, and Helicase Inhibitots. 2015. https://www.informedhorizons.com].

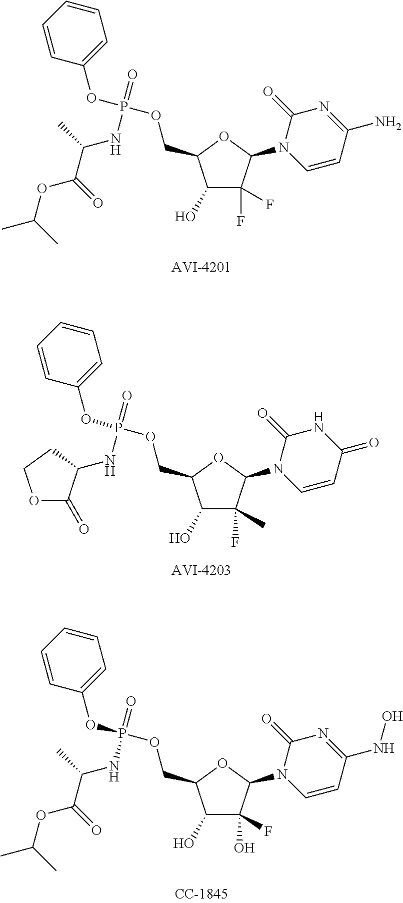

Should be noted that in this case the inhibitory activity of HCV NS5b RNA-polymerase phosphoramidate inhibitors Sovaldi®, AVI-4201 AVI-4203 and CC-1845 by orders of magnitude higher than that of the corresponding nucleoside: PSI-6206, Gemcitabine and 2′-C-Methylcytidine.

Other guanosine nucleotide prodrugs have been investigated, also employing the phosphoramidate prodrug moiety in an attempt to leverage liver targeting. Thus, PSI-353661 demonstrated potent inhibition in the replicon assay (EC₉₀=0.008 μM→1000-fold increase in potency compared to the guanosine analogue—β-D-2′-deoxy-2′-R-fluoro-2′-β-C-methylguanosine (Table 1)) and a novel resistance profile similar to PSI-3 52938, but was never progressed into clinical development [W. Clung et al. Discovery of PSI-353661, a Novel Purine Nucleotide. ACS Med. Chem. Lett. 2011. 2. 130-135.]. The structurally related pro-drugs IDX-184 (EC₅₀=0.4 μM) [X.-J. Zhou. Et al. Safety and Pharmacokinetics of IDX184, a Liver-Targeted Nucleotide Polymerase Inhibitor of Hepatitis C Virus, in Healthy Subjects Antimicrob. Agents Chemother. 2011, 55, 76-81. J. Lalezari, et al. Short-Term Monotherapy with IDX184, a Liver-Targeted Nucleotide Polymerase Inhibitor, in Patients with Chronic Hepatitis C Virus Infection. Antimicrob. Agents Chemother. 2012. 56, 6372-6378.] and INX-08189 (BMS-986094, EC₅₀=0.010 μM) [C. McGuigan et al. Phosphorodiamidates as a Promising New Phosphate Prodrug Motif for Antiviral Drug Discovery: Application to Anti-HCV Agents. J. Med. Chem. 2011, 54, 8632-8645. J. H. Vemachio et al. INX-08189, a phosphoramidate prodrug of 6-O-methyl-2′-C-methyl guanosine, is a potent inhibitor of hepatitis C virus replication with excellent pharmacokinetic and pharmacodynamic properties. Antimicrob. Agents Chemother. 2011. 55, 1843-1851.], each producing an identical triphosphate, were progressed into the clinic, but severe cardiovascular toxicity associated with INX-08189 resulted in discontinuation of development for both compounds [J. J. Arnold et al. Sensitivity of Mitochondrial Transcription and Resistance of RNA Polymerase II Dependent Nuclear Transcription to Antiviral Ribonucleosides. PLOS Pathog. 2012. 8, DOI: 10.1371/journal.ppat. 1003030.]. The severe nature of the cardiovascular toxicity seen with INX-08189 seems to have curtailed the interest in developing a guanosine nucleoside for treating HCV patients.

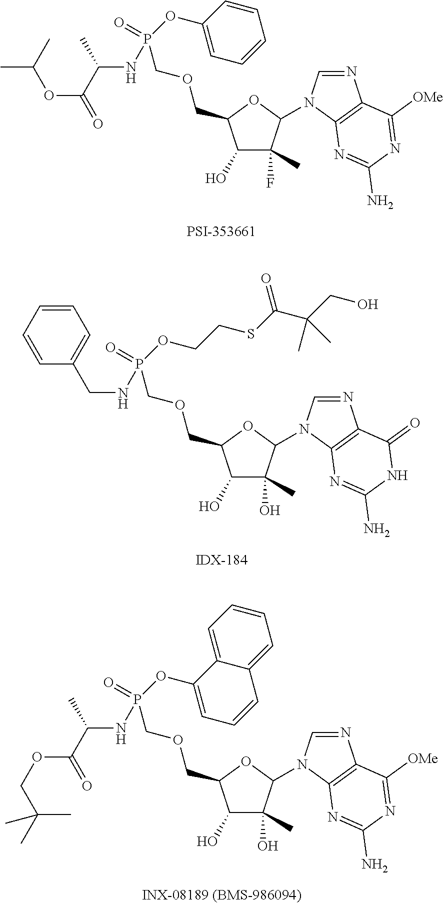 [SMILES: COc1nc(N)nc2c1ncn2C1O[C@H](COCP(=O)(N[C@@H](C)C(=O)OC(C)C)Oc2ccccc2)[C@@H](O)[C@@]1(C)F ; CC(C)(CO)C(=O)SCCOP(=O)(COC[C@H]1OC(n2cnc3c(=O)[nH]c(N)nc32)[C@](C)(O)[C@@H]1O)NCc1ccccc1 ; COc1nc(N)nc2c1ncn2C1O[C@H](COCP(=O)(N[C@@H](C)C(=O)OCC(C)(C)C)Oc2cccc3ccccc23)[C@@H](O)[C@@]1(C)O]

HBV is a DNA virus in the Hepadnaviridate family. It is estimated that 400 million individuals are infected with HBV worldwide. The current standard of care for treatment of HBV is long-term nucleos(t)ide therapy. The nucleos(t)ides approved for treating HBV infection include lamivudine, adefovir dipivoxil, entecavir, telbivudine, and TDF. Entecavir and TDF are the most widely prescribed of these agents. Long-term use of entecavir leads to resistance in a significant patient population and TDF is associated with nephrotoxicity and bone loss [D. Grimm et al. HBV life cycle and novel drug targets. Hepatol. Int. 2011. 5. 644-653. G. Borgia, I. Gentile. Treating chronic hepatitis B: today and tomorrow. Curr. Med. Chem. 2006. 13. 2839-2855.]. However, continued use of nucleos(t)ide therapy has been associated with reduction in liver fibrosis demonstrating that suppression of viral replication has positive long-term value [T. T. Chang et al. Long-term entecavir therapy results in the reversal of fibrosis/cirrhosis and continued histological improvement in patients with chronic hepatitis B. Hepatology 2010. 52, 886-893. P. Marcellin et al. Regression of cirrhosis during treatment with tenofovir disoproxil fumarate for chronic hepatitis B: a 5-year open-label follow-up study. Lancet 2013, 381, 468-475.].

Even with the success of existing nucleos(t)ide HBV therapy, work has continued in an effort to identify, novel inhibitors that may provide additional benefit relative to the existing agents, and several of the anti-HIV agents mentioned above have also been assessed for us in treating HBV infection [C. A. Geng et al. Small-molecule inhibitors for the treatment of hepatitis B virus documented in patents. Mini Rev. Med. Chem. 2013. 13, 749-776.].

Recently, preparation of the 2′-fluoro-6′-methylene-carbocyclic adenosine (FMCA) (EC₅₀=0.55 μM), which borrowed the 6′-methylene-carbocyclic nucleus of entecavir, led to a potent inhibitor of HBV replication that was also active against the lamivudine-entecavirresistant clone (L180M+M204V+S202G) [R. K. Rawal et al. 2′-Fluoro-6′-methylene-carbocyclic adenosine phosphoramidate (FMCAP) prodrug: In vitro anti-HBV activity against the lamivudine-entecavir resistant triple mutant and its mechanism of action. Bioorg. Med. Chem. Lett. 2013. 23, 503-506.]. Furthermore, preparation of the corresponding 5′-phosphoramidate of FMCA resulted in a compound that was 10-fold more potent than FMCA against both the wild-type (EC₅₀=0.62 μM) and resistant mutant (EC₅₀=0.054 μM) [R. K. Rawal et al. 2′-Fluoro-6′-methylene-carbocyclic adenosine phosphoramidate (FMCAP) prodrug: In vitro anti-HBV activity against the lamivudine-entecavir resistant triple mutant and its mechanism of action. Bioorg. Med. Chem. Lett. 2013, 23, 503-506.].

It is also known that the phosphoramidate conjugates of clevudine (EIDD-02173) retained potent anti-HBV activity in cell culture models of infection. The phosphoramidate moiety successfully delivered clevudine-5′-monophosphate to the liver while significantly decreasing non-liver organ exposure. Selective targeting of the liver could potentially lead to a decrease in the off-target effects related to clevudine in humans. [G. R. Bluemling et al. Targeted Delivery of Clevudine-5′-Monophosphate to the Liver After Oral Administration of a Clevudine-5′-Phosphoramidate Conjugate to Rats for the Treatment of HBV Infections. Global Antiviral Journal 2015, 11, Suppl. 3: HEP DART 2015: Abstr. 104, P. 97].

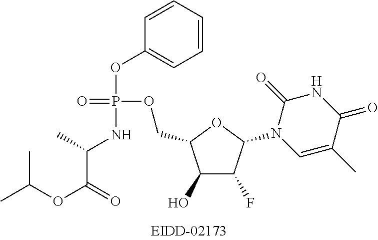 [SMILES: Cc1cn([C@H]2O[C@@H](COP(=O)(N[C@@H](C)C(=O)OC(C)C)Oc3ccccc3)[C@H](O)[C@H]2F)c(=O)[nH]c1=O]

Gemcitabin-5′-phosphoramidate (NUC-1031) [M. Slusarczyk et al. Application of ProTide Technology to Gemcitabine: A Successful Approach to Overcome the Key Cancer Resistance Mechanisms Leads to a New Agent (NUC-1031) in Clinical Development. J. Med. Chem. 2014, 57, 1531-1542] showed a high anti-cancer activity. In particular NUC-1031 significantly reduced tumor volume in vivo in xenograft models of human pancreatic cancer. Important to note that activation of NUC-1031 is much less dependent on the nucleoside transporters and deoxycytidine than gemcitabine. In addition, NUC-1031 is resistant to degradation cytidine deaminase unlike gemcitabine.

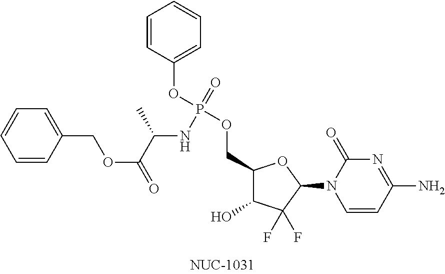 [SMILES: C[C@H](NP(=O)(OC[C@H]1O[C@@H](n2ccc(N)nc2=O)C(F)(F)[C@@H]1O)Oc1ccccc1)C(=O)OCc1ccccc1]

It should be noted that the structure of phosphoramidate moiety has a significant impact on the stability of phosphoramidate nucleosides in various media, their pharmacokinetics, bioavailability, distribution in body organs and the selectivity of their action [M. J. Sofia et al. 2010. P. Wang et al. Phosphoramidate prodrugs of (−)-β-D-(2R,4R)-dioxolane-thymine (DOT) as potent anti-HIV agents. Antiviral Chem. Chemotherapy 2012, 22, 217-238. L. Bondada et al. Adenosine Dioxolane Nucleoside Phosphoramidates as Antiviral Agents for Human Immunodeficiency and Hepatitis B Viruses. ACS Med. Chem. Lett. 2013, 4, 747-751. M. Slusarczyk et al. Application of ProTide Technology to Gemcitabine: A Successful Approach to Overcome the Key Cancer Resistance Mechanisms Leads to a New Agent (NUC-1031) in Clinical Development. J. Med. Chem. 2014, 57, 1531-1542.].

So far synthesis of new phosphoramidate nucleoside prodrugs and their use as chemotherapeutic agents for the treatment of viral diseases and cancer are highly relevant.

Is important to note also that until now were unknown nucleoside-containing macroheterocyclic phosphoramidates and their use for treating viral and cancerous diseases.

### SUMMARY OF THE INVENTION

The present invention is directed toward novel chemotherapeutic agents which are containing phosphoramidate and nucleoside moieties of the general formula 1

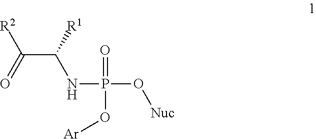

their stereoisomers, isotope-enriched analogues, pharmaceutically acceptable salts, hydrates, solvates, or crystalline or polymorphic forms thereof, wherein:
Ar is aryl or hetaryl;
R¹ is H or CH₃;
R² is the substituent selected from OCH₂CH═CH₂, OCH₂CH≡CH, OCH₂CH₂CH₂OCH₃,

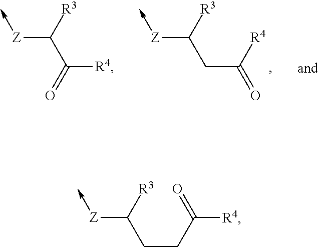

R³ is H or CH₃;
R⁴ is OH, OR⁵ or NR⁶R⁷;
R⁵ is C₁-C₄-alkyl;
R⁶ and R⁷ are not necessarily the same substituents selected from H or CH₃;

### Z═O or NH;

an arrow (→) indicates the place of substituent connection;

### Nuc is

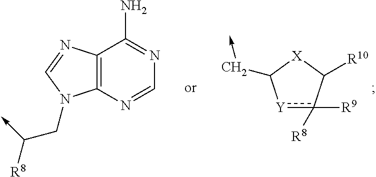

R⁸ and R⁹ are not necessarily the same substituents selected from H, F, Cl, CH₃, OH provided when continuous line and its accompanying dotted line ( [symbol] ) together are the single carbon-carbon (C—C) bond or R⁸ and R⁹ are hydrogen provided when continuous line and its accompanying dotted line ( [symbol] ) together are the double carbon-carbon bond (C═C);
R¹⁰ is the substituent selected from R^{10.1}-R^{10.5};

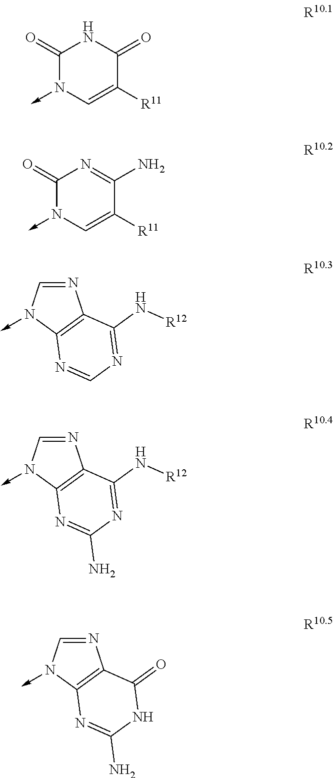

R¹¹ is the substituent selected from H, F, Cl, CH₃ or CF₃;
R¹² is hydrogen, C₁-C₄-alkyl or C₃-C₆-cycloalkyl;
X is oxygen or ethanediyl-1,1 (C═CH₂);
Y is O, S, CH₂, or HO—CH group provided when continuous line and its accompanying dotted line ( [symbol] ) together are the single carbon-carbon (C—C) bond or Y is CH group provided when continuous line and its accompanying dotted line ( [symbol] ) together are the double carbon-carbon bond (C═C); and compound of the general formula 1, stereoisomers, isotope-enriched analogues, pharmaceutically acceptable salts, hydrates, solvates, or crystalline or polymorphic forms thereof, wherein:
Ar is aryl or hetaryl;
R¹ is H or CH₃;
R² is isopropyl;

### Nuc is

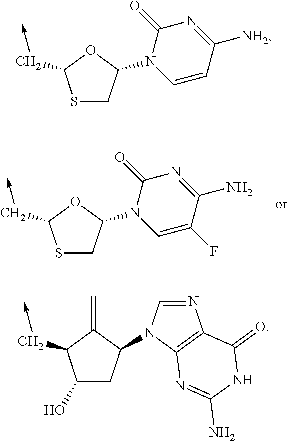

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, and unless otherwise specified, refers to substituted or unsubstituted phenyl (Ph), biphenyl, or naphthyl; preferably the term aryl refers to substituted or unsubstituted phenyl. The aryl group can be substituted with one or more moieties selected from among hydroxyl, F, Cl, Br, I, amino, alkylamino, arylamino, alkoxy, aryloxy, nitro, cyano, sulfonic acid, sulfate, phosphonic acid, phosphate, and phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999.

The term “heteroaryl”, as used herein, refers to a mono- or polycyclic aromatic radical having one or more ring atom selected from S, O and N; and the remaining ring atoms are carbon. Heteroaryl includes, but is not limited to, pyridinyl, pyrazinyl, pyrimidinyl, quinolinyl, isoquinolinyl, benzimidazolyl, benzooxazolyl, or quinoxalinyl.

The term “alkyl” as used herein, refers to saturated, straight- or branched-chain hydrocarbon radicals containing from one to six carbon atoms. The examples of C₁-C₆ alkyl radicals include, but are not limited to, methyl, ethyl, propyl, isopropyl, n-butyl, and tert-butyl.

“Lower alkyl” refers to an unbranched or branched alkyl chain comprising 1-4 carbon atoms.

The term “alkoxy” refers to an —O-alkyl group or an —O-cycloalkyl group, wherein alkyl and cycloalkyl are as defined above. Examples of —O-alkyl groups include, but are not limited to, methoxy, ethoxy, n-propyloxy, i-propyloxy, n-butyloxy, i-butyloxy, t-butyloxy. “Lower alkoxy” as used herein denotes an alkoxy group with a “lower alkyl” group as previously defined. “C₁₋₁₀ alkoxy” refers to an —O-alkyl wherein alkyl is C₁₋₁₀. Examples of —O-cycloalkyl groups include, but are not limited to, —O-c-propyl, —O-c-butyl, —O-c-pentyl, and —O-c-hexyl.

The term “cycloalkyl” as used herein, refers to carbocyclic ring system containing from 3 to six carbon atoms. The examples of C₃-C₆ cycloalkyl radicals include, but are not limited to, cyclopropyl, cyclobutyl, cyclopentyl, cyclohexyl.

The term “optionally substituted” means that the group referred to can be substituted at one or more positions by any one or any combination of the radicals.

The term “Active component” (drug substance) refers to a physiologically active compound of synthetic or other (biotechnological, vegetable, animal, microbicidal, and so on) origins exhibiting a pharmacological activity, which is an active ingredient of the pharmaceutical composition employed in production.

The term “Leaving group” as used herein refers to a weakly basic chemical entity that is readily released from carbon, and takes the pair of bonding electrons binding it with said carbon atom. Leaving groups are chemical functional groups that can be displaced from carbon atoms by nucleophilic substitution. Examples include, but are not limited to, alkylsulfonates, substituted alkylsulfonates, arylsulfonates, substituted arylsulfonates, heterocyclicsulfonates, trichloroacetimidate, alkoxide, and aryloxide groups. Preferred leaving groups include, but are not limited to, chloride, bromide, iodide, para-nitrobenzenesulfonate (nosylate), para-(2,4-dinitroanilino)benzenesulfonate, benzenesulfonate, methylsulfonate (mesylate), para-methylbenzenesulfonate (tosylate), para-bromobenzenesulfonate, trifluoromethylsulfonate, 2,2,2-trifluoroethanesulfonate, imidazolesulfonate, trichloroacetimidate, trifluoroacetate and other acylates, alkoxide, and aryloxide, i.e., 4-nitrophenoxide, pentafluorophenoxide, and 2,4,6-trichlorophenoxide.

The term “Protective Groups”—the synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include the 2-tetrahydropyranyl group, 2-(bisacetoxyethoxy)methyl group, trityl group, trichloroacetyl group, carbonate-type blocking groups such as benzyloxycarbonyl (Cbz), trialkylsilyl groups, examples of such being trimethylsilyl, tert-butyldimethylsilyl, tert-butyldiphenylsilyl, phenyldimethylsilyl, triisopropylsilyl and thexyldimethylsilyl, ester groups such as formyl, C₁-C₁₀ alkanoyl (optionally mono-, di- or tri-enriched with C₁-C₆ alkyl, C₁-C₆ alkoxy, halo, aryl, aryloxy or haloaryloxy), and the like, the aroyl group (including optionally mono-, di- or tri-enriched on the ring carbons with halo, C₁-C₆ alkyl, C₁-C₆ alkoxy wherein aryl is phenyl, 2-furyl, and the like), carbonates, sulfonates, and ethers such as benzyl, p-methoxybenzyl, methoxymethyl, 2-ethoxyethyl, benzyloxymethyl (BOM) group, and etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by [J. W. Barton, “Protective Groups In Organic Chemistry”, J. G. W. McOmie, Ed., Plenum Press, New York, N.Y., 1973, and G. M. Wuts, T. W. Greene, “Protective Groups in Organic Synthesis”, John Wiley & Sons Inc., Hoboken, N.J., 2007], which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include acetyl (Ac), trifluoroacetyl, benzoyl (Bz), Boc, Cbz, trityl, TBDMS, DMTr, and benzyl (Bn). See also [G. M. Wuts, T. W. Greene, “Protective Groups in Organic Synthesis”, John Wiley & Sons Inc., Hoboken, N.J., 2007], and related publications.

The reduction “Boc” as used herein, refers to the tert-butoxycarbonyl protecting group.

The reduction “Cbz” as used herein, refers to the benzyloxycarbonyl protecting group.

The reduction “TBDMS” as used herein, refers to the tert-butyldimethylsilyl protecting group.

“Medicament” is a compound (or a mixture of compounds as a pharmaceutical composition) and a preparation of medicaments in the form of tablets, capsules, injections, ointments, and other ready forms intended for restoration, improvement, or modification of physiological functions in humans and animals and for the treatment and prophylaxis of diseases, for diagnostics, anesthesia, contraception, cosmetology, and so on.

“Therapeutic cocktail” represents a simultaneously administered combination of two or more medicaments exhibiting a different mechanism of pharmacological action and directed to various biotargets taking part in the disease process.

“Pharmaceutical composition” means a composition comprising a compound of general formula 1 and at least one component selected from a group consisting of pharmaceutically acceptable and pharmacologically compatible fillers, solvents, diluents, carriers, auxiliaries, distributors and excipients, delivery agents, such as preservatives, stabilizers, fillers, disintegrators, moisteners, emulsifiers, suspending agents, thickeners, sweeteners, flavouring agents, aromatizing agents, antibacterial agents, fungicides, lubricants, and prolonged delivery controllers, choice and suitable proportions of which depend on the nature and way of administration and dosage. Examples of suitable suspending agents are ethoxylated isostearyl alcohol, polyoxyethene, sorbitol and sorbitol ether, microcrystalline cellulose, aluminum metahydroxide, bentonite, agar-agar and tragacanth, and mixtures thereof.

Protection against microorganisms can be provided by various antibacterial and antifungal agents, such as, for example, parabens, chlorobutanole, sorbic acid, and similar compounds. Composition may also comprise isotonic agents, such as, for example, sugar, sodium chloride, and similar compounds. The prolonged effect of a composition may be achieved by agents slowing down the absorption of the active ingredient, for example, aluminum monostearate or gelatine. Examples of suitable carriers, solvents, diluents, and delivery agents include water, ethanol, polyalcohols and mixtures thereof, natural oils (such as olive oil), and organic esters (such as ethyl oleate) for injections. Examples of fillers are lactose, milk sugar, sodium citrate, calcium carbonate, calcium phosphate, and the like. The examples of disintegrators and distributors are starch, alginic acid and its salts, and silicates.

The examples of suitable lubricants are magnesium stearate, sodium lauryl sulfate, talc, and high molecular weight polyethylene glycol. A pharmaceutical composition for peroral, sublingval, transdermal, intramuscular, intravenous, subcutaneous, and local or rectal administration of the active ingredient, alone or in combination with another active compound, may be administered to humans and animals in standard administration form, or in a mixture with traditional pharmaceutical carriers. Suitable standard administration forms include peroral forms such as tablets, gelatin capsules, pills, powders, granules, chewing gums, and peroral solutions or suspensions; sublingval and transbuccal administration forms; aerosols; implants; local, transdermal, subcutaneous, intramuscular, intravenous, intranasal or intraocular forms, and rectal administration forms.

The compounds or salts of the present invention may also be used in the form of prodrugs.

The compounds of the invention may comprise asymmetrically substituted carbon and phosphorus atoms known as chiral centers. These compounds may exist, without limitation, as single stereoisomers or racemic mixtures. Compounds identified herein as single stereoisomers are meant to describe compounds that are present in a form that is substantially free from other stereoisomers (e.g., substantially free from other enantiomers or diastereomers). By “substantially free” it is meant that at least 80% of the compound in a composition is the described stereoisomer; preferably, at least 90% of the compound in a composition is the described stereoisomer; and, more preferably, at least 95%, 96%, 97%, 98%, or 99% of the compound in a composition is the described stereoisomer. Where the stereochemistry of a chiral carbon is not specified in the chemical structure of a compound, the chemical structure is intended to encompass compounds containing either stereoisomer of the chiral center. Individual stereoisomers of the compounds of this invention can be prepared using a variety of methods known in the art. These methods include, but are not limited to, stereospecific synthesis, chromatographic separation of diastereomers, chromatographic resolution of enantiomers, conversion of enantiomers in an enantiomeric mixture to diastereomers followed by chromatographic separation of the diastereomers and regeneration of the individual enantiomers, and enzymatic resolution.

Stereospecific synthesis typically involves the use of appropriate optically pure (enantiomerically pure) or substantially optically pure materials and synthetic reactions that do not cause racemization or inversion of stereochemistry at the chiral centers. Mixtures of stereoisomers of compounds, including racemic mixtures, resulting from a synthetic reaction may be separated, for example, by chromatographic techniques as appreciated by those of ordinary skill in the art. Chromatographic resolution of enantiomers can be accomplished by using chiral chromatography resins, many of which are commercially available. In a non-limiting example, racemate is placed in a solution and loaded onto the column containing a chiral stationary phase. Enantiomers can then be separated by HPLC. The resolution of enantiomers can also be improved by converting enantiomers in a mixture to diastereomers by reaction with chiral auxiliaries. The resulting diastereomers can be separated by column chromatography or crystallization/re-crystallization. This technique is useful when the compounds to be separated contain a carboxyl, amino or hydroxyl group that will form a salt or a covalent bond with the chiral auxiliary. Non-limiting examples of suitable chiral auxiliaries include chirally pure amino acids, organic carboxylic acids, or organosulfonic acids. Once the diastereomers are separated by chromatography, individual enantiomers can be regenerated. Frequently, the chiral auxiliary can be recovered and used again.

Enzymes such as esterases, phosphatases, or lipases can be useful for the resolution of derivatives of enantiomers in an enantiomeric mixture. For example, an ester derivative of a carboxyl group in the compounds to be separated can be treated with an enzyme, which selectively hydrolyzes only one of the enantiomers in the mixture. The resulting enantiomerically pure acid can then be separated from the unhydrolyzed ester.

Alternatively, salts of enantiomers in a mixture can be prepared using any suitable method known in the art, including treating the carboxylic acid with a suitable optically pure base, such as alkaloids or phenethylamine, followed by precipitation or crystallization/re-crystallization of the enantiomerically pure salts. Methods suitable for the resolution/separation of a mixture of stereoisomers, including racemic mixtures, can be found in [Jacques et al., Enantiomers, racemates, and resolutions, 1981, John Wiley and Sons, New York, N.Y.].

In addition, where a compound exists in various tautomeric forms, a recited compound is not limited to any one specific tautomer, but rather is intended to encompass all tautomeric forms. The compounds of the invention may exist in different stable conformational forms, which may be separable. Torsional asymmetry due to restricted rotations about an asymmetric single bond, for example, because of steric hindrance or ring strain, may permit separation of different conformers. The invention encompasses each conformational isomer of these compounds and mixtures thereof.

The compounds of the present invention are generally described herein using standard nomenclature. For a recited compound having asymmetric center(s), it should be understood that all of the stereoisomers of the compound and mixtures thereof are encompassed in the present invention unless otherwise specified. Non-limiting examples of stereoisomers include enantiomers, diastereomers, and cis-transisomers. Where a recited compound exists in various tautomeric forms, the compound is intended to encompass all tautomeric forms.

The number of carbon atoms in a hydrocarbyl moiety can be indicated by the prefix “Cₓ-C_{y}” where x is the minimum and y is the maximum number of carbon atoms in the moiety. Thus, for example, “C₁-C₆alkyl” refers to an alkyl substituent containing from 1 to 6 carbon atoms. If a linking element in a depicted structure is a bond, then the element left to the linking element is joined directly to the element right to the linking element via a covalent bond. If two or more adjacent linking elements in a depicted structure are bonds, then the element left to these linking elements is joined directly to the element right to these linking elements via a covalent bond.

When a chemical formula is used to describe a moiety, the dash(es) indicates the portion of the moiety that has the free valence(s). If a moiety is described as being “optionally substituted”, the moiety may be either substituted or unsubstituted. If a moiety is described as being optionally substituted with up to a particular number of non-hydrogen radicals, said moiety may be either unsubstituted, or substituted by up to that particular number of non-hydrogen radicals, or by up to the maximum number of substitutable positions on the moiety, whichever is less. Thus, for example, if a moiety is described as a heterocycle optionally substituted with up to three non-hydrogen radicals, then any heterocycle with less than three substitutable positions will be optionally substituted by up to only as many non-hydrogen radicals as the heterocycle has substitutable positions.

The term “pharmaceutically acceptable” is used adjectivally to mean that the modified noun is appropriate for use as a pharmaceutical product or as a part of a pharmaceutical product.

The term “therapeutically effective amount” refers to the total amount of each active substance that is sufficient to show a meaningful patient benefit, e.g., a reduction in viral load.

The term “prodrug” refers to derivatives of the compounds of the invention which have chemically or metabolically cleavable groups and become, by solvolysis or under physiological conditions, the compounds of the invention, which are pharmaceutically active in vivo. A prodrug of a compound may be formed in a conventional manner by reaction of a functional group of the compound (such as an amino, hydroxy or carboxy group). Prodrugs often offer advantages of solubility, tissue compatibility, or delayed release in mammals (see, Bungard, H., Desing of products, pp. 7-9, 21-24, Elsevier, Amsterdam 1985). Prodrugs include acid derivatives well known to practitioners in the art, such as, for example, esters prepared by reaction of the parent acidic compound with a suitable alcohol, or amides prepared by reaction of the parent acid compound with a suitable amine. Examples of prodrugs include, but are not limited to, acetate, formate, benzoate or other acylated derivatives of alcohol or amine functional groups within the compounds of the invention.

The term “solvate” refers to a physical association of a compound of this invention with one or more solvent molecules, whether organic or inorganic. This physical association often includes hydrogen bonding. In certain instances, the solvate will be capable of isolation, for example, when one or more solvent molecules are incorporated in the crystal lattice of the crystalline solid. “Solvate” encompasses both solution-phase and isolable solvates. Exemplary solvates include, but are not limited to, hydrates, ethanolates, and methanolates.

The present disclosure will now be described in connection with certain embodiments, which are not intended to limit its scope. On the contrary, the present disclosure covers all alternatives, modifications, and equivalents as can be included within the scope of the claims. Thus, the following examples, which include specific 10 embodiments, will illustrate one practice of the present disclosure, it being understood that the examples are for the purposes of illustration of certain embodiments and are presented to provide what is believed to be the most useful and readily understood description of its procedures and conceptual aspects.

### DETAILED DESCRIPTION OF THE INVENTION

An aspect of the invention is phosphoramidate nucleoside prodrug of the general formula 1, a stereoisomer, isotope-enriched analogue, pharmaceutically acceptable salt, hydrate, solvate, or crystalline or polymorphic form thereof,

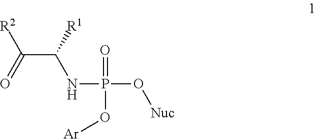

wherein:
Ar is aryl or hetaryl;
R¹ is H or CH₃;
R² is the substituent selected from OCH₂CH═CH₂, OCH₂CH≡CH, OCH₂CH₂CH₂OCH₃,

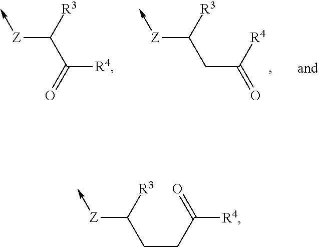

R³ is H or CH₃;
R⁴ is OH, OR⁵ or NR⁶R⁷;
R⁵ is C₁-C₄-alkyl;
R⁶ and R⁷ are not necessarily the same substituents selected from H or CH₃;

### Z═O, or NH;

an arrow (→) indicates the place of substituent connection;

### Nuc is

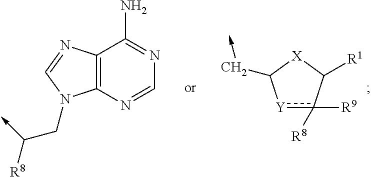

R⁸ and R⁹ are not necessarily the same substituents selected from H, F, Cl, CH₃, OH provided when continuous line and its accompanying dotted line ( [symbol] ) together are the single carbon-carbon (C—C) bond or R⁸ and R⁹ are hydrogen provided when continuous line and its accompanying dotted line ( [symbol] ) together are the double carbon-carbon bond (C═C);
R¹⁰ the substituent selected from R^{10.1}-R^{10.5};

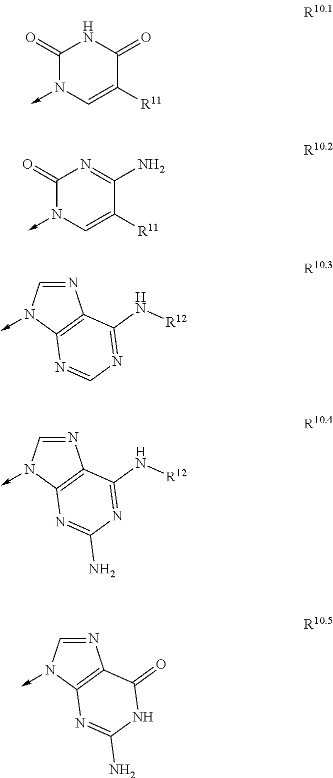

R¹¹ is the substituent selected from H, F, Cl, CH₃ or CF₃;
R¹² is hydrogen, C₁-C₄-alkyl or C₃-C₆-cycloalkyl;
X is oxygen or ethanediyl-1,1 (C═CH₂);
Y is O, S, CH₂, or HO—CH group provided when continuous line and its accompanying dotted line ( [symbol] ) together are the single carbon-carbon (C—C) bond or Y is CH group provided when continuous line and its accompanying dotted line ( [symbol] ) together are the double carbon-carbon bond (C═C). Preferred phosphoramidate is compound of general formula 1.1 and 1.2 a stereoisomer, isotope-enriched analogue, pharmaceutically acceptable salt, hydrate, solvate, or crystalline or polymorphic form thereof,

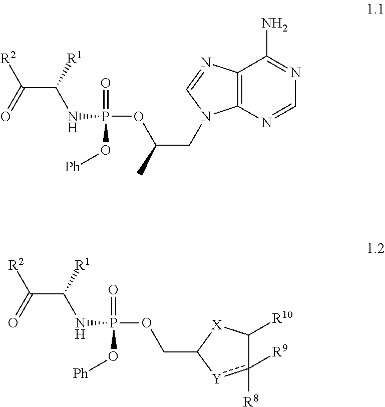

wherein: R¹, R², R⁸, R⁹, R¹⁰, X, Y, and continuous line and its accompanying dotted line ( [symbol] ) have the above mentioned meaning;
and compound of the general formula 1, stereoisomers, isotope-enriched analogues, pharmaceutically acceptable salts, hydrates, solvates, or crystalline or polymorphic forms thereof, wherein:
Ar is aryl or hetaryl;
R¹ is H or CH₃;
R² is isopropyl;

### Nuc is

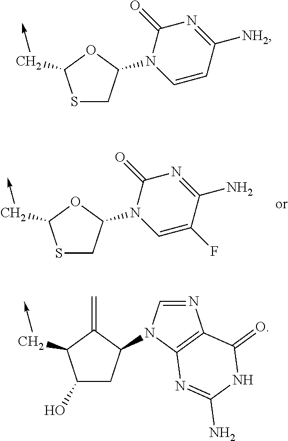

More preferred phosphoramidate are: allyl (S)-2-{[(R)-1-methyl-2-(6-amino-purin-9-yl)-ethoxy]-phenoxy-phosphorylamino}-propanoate (1.1(1)), 2-methoxy-ethyl (S)-2-{[(R)-1-methyl-2-(6-amino-purin-9-yl)-ethoxy]-phenoxy-phosphorylamino}-propanoate (1.1(2)), (S)-1-ethoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(1)), (S)-1-isopropyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(2)), (S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)), (S)-1-ethoxycarbonyl-cyclopropyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(4)), isopropyl (S)-2-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionylamino)-propanoate (1.2(5)), (S)-1-carboxy-ethyl (S)-2-{([(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino)}-propionate (1.2(6)), (S)-1-carbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(7)), (S)-1-dimethylcarbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(8)), allyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(9)), prop-2-ynyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(10)), 2-methoxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(11)), (S)-1-isopropoxycarbonyl-ethyl (S)-2-{(R)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(12)), (S)-1-isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13)), benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), 4-((S)-2-[(2R,3R,4R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butyric acid (1.2(15)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(16)), (S)-1-isopropoxycarbonyl-ethyl S)-2-{(S)-[(2R,3S,5R)-5-(2,4-dioxo-5-methyl-3,4-dihydro-2H-pyrimidin-1-yl)-3-hydroxy-tetrahydro-furan-2-ylmethoxy]-phenoxy-phosphorylamino}-propanoate (1.2(17)), (S)-1-isopropoxycarbonyl-ethyl S)-2-{(S)-[(2R,3S,5R)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(18)), (S)-1-isopropoxycarbonyl-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(19)), (S)-1-isopropoxycarbonyl-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(20)), (S)-1-isopropoxycarbonyl-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(21)), (S)-1-isopropoxycarbonyl-ethyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(22)), allyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(23)), prop-2-ynyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(24)), 2-methoxymethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(25)), allyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(26)), (S)-1-isopropoxycarbonyl-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(27)), (S)-1-isopropoxycarbonyl-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(28)), (S)-1-isopropoxycarbonyl-ethyl (S)-2-{(S)-[(2R,3R,4R,5R)-5-(2,4-doxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(29)), prop-2-ynyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(30)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(31)), prop-2-ynyl ester (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(32), 2-methoxy-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(33), allyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(34), allyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(35), prop-2-ynyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(36)), 2-methoxy-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(37)), allyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(38)), prop-2-ynyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(39)), 2-methoxy-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(40)), allyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(41)), prop-2-ynyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(42)), 2-methoxy-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(43)), allyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino)}-propanoate (1.2(44)), prop-2-ynyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(45)), 2-methoxy-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(46)), allyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(47)), prop-2-ynyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(48)), 2-methoxy-ethyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(49)), (S)-1-isopropoxycarbonyl-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(50)), allyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(51)), prop-2-ynyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(52)), 2-methoxy-ethyl(S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(53)), (S)-1-isopropoxycarbonyl-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(54)), (S)-1-isopropoxycarbonyl-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(55)), (S)-1-isopropoxycarbonyl-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(56)), (S)-1-isopropoxycarbonyl-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(57)), allyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(58)), prop-2-ynyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(59)), 2-methoxymethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(60)), allyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (11.2(61))), prop-2-ynyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(62)), 2-methoxy-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(63)), allyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(64)), prop-2-ynyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(65)), 2-methoxy-ethyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(66)), allyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(67)), prop-2-ynyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(68)), 2-methoxy-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(69)), allyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(70)), prop-2-ynyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(71)), 2-methoxy-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(72)), allyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(73)), prop-2-ynyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(74)), 2-methoxy-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(75)), (S)-1-isopropoxycarbonyl-ethyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(76)), allyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(77)), prop-2-ynyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(78)), 2-methoxy-ethyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(79)), (S)-1-isopropoxycarbonyl-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(80)), (S)-1-isopropoxycarbonyl-ethyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(81)), (S)-1-isopropoxycarbonyl-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(82)), allyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(83)), prop-2-ynyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(84)), 2-methoxy-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(85)), allyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(86)), prop-2-ynyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(87)), 2-methoxy-ethyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(88)), allyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(89)), prop-2-ynyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(90)), 2-methoxy-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(91)), (S)-1-isopropoxycarbonyl-ethyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1.2(92)), (S)-1-isopropoxycarbonyl-ethyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1.2(93)), allyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(94)), prop-2-ynyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(95)), 2-methoxy-ethyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(96)), allyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(97)), prop-2-ynyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(98)), 2-methoxy-ethyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(99)), isopropyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1(1)), isopropyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1(2)), isopropyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1(3)), their stereoisomers, isotope-enriched analogues, pharmaceutically acceptable salts, hydrates, solvates, or crystalline or polymorphic forms thereof

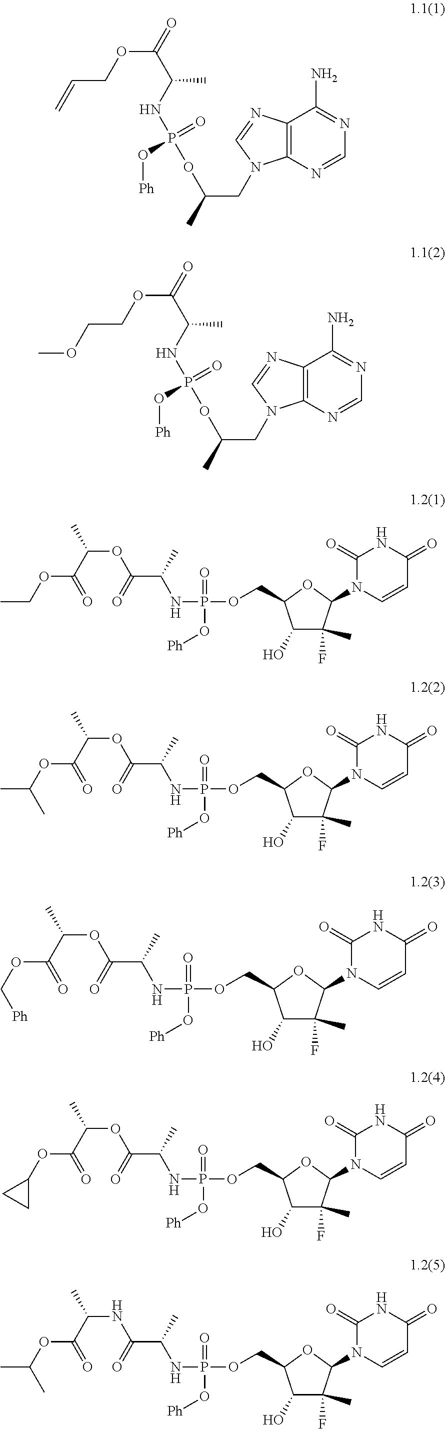

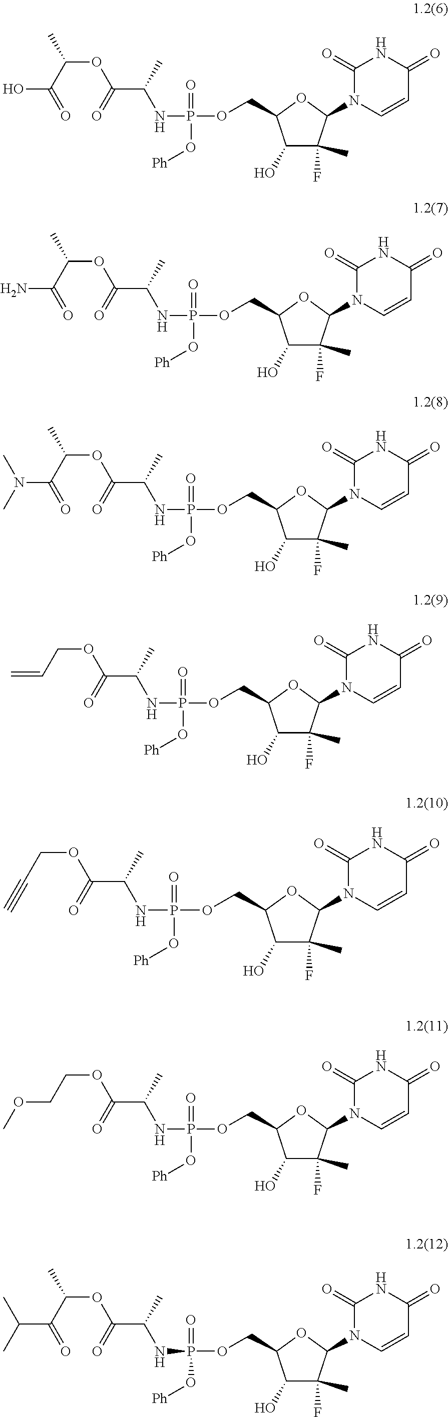

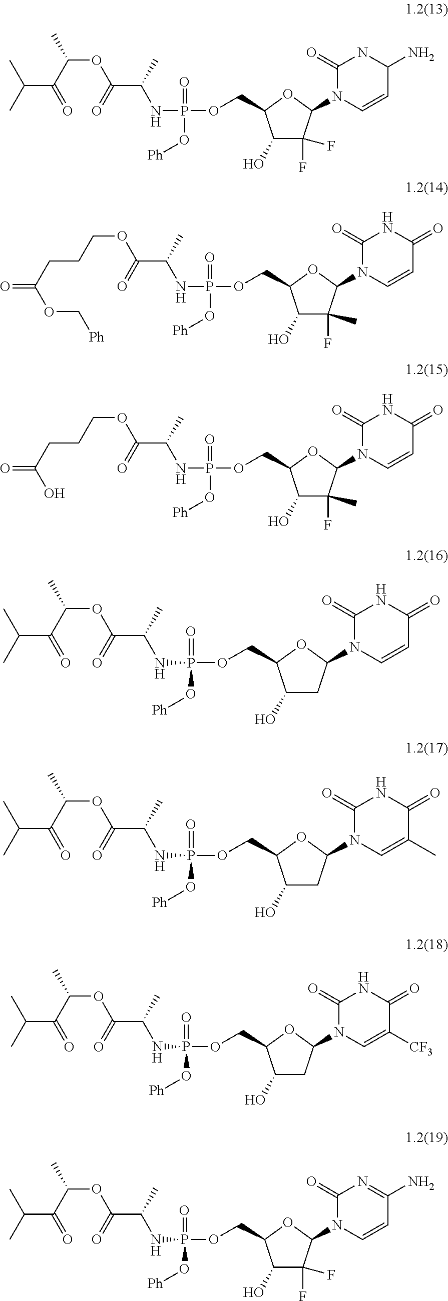

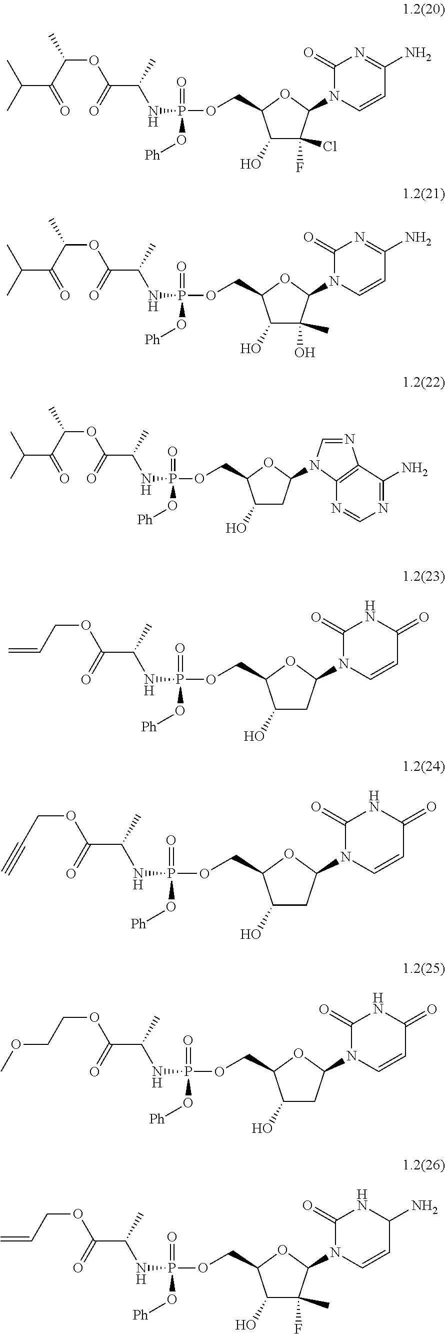

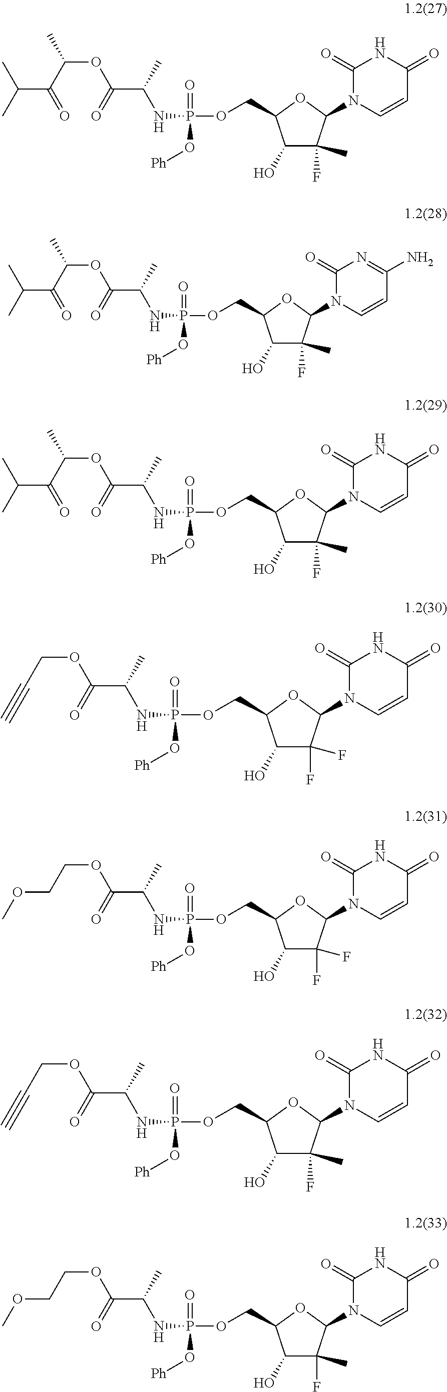

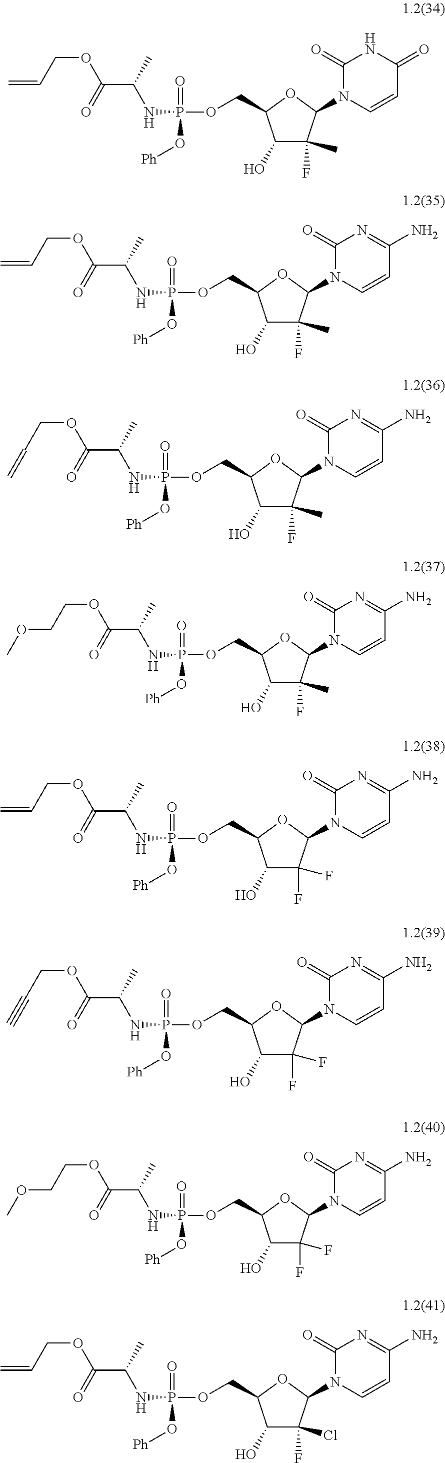

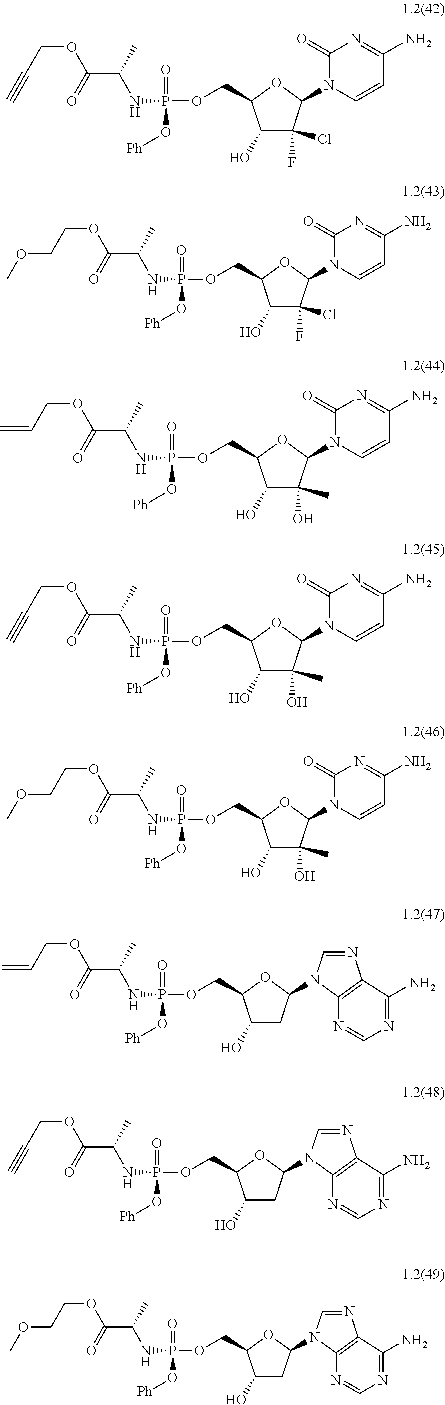

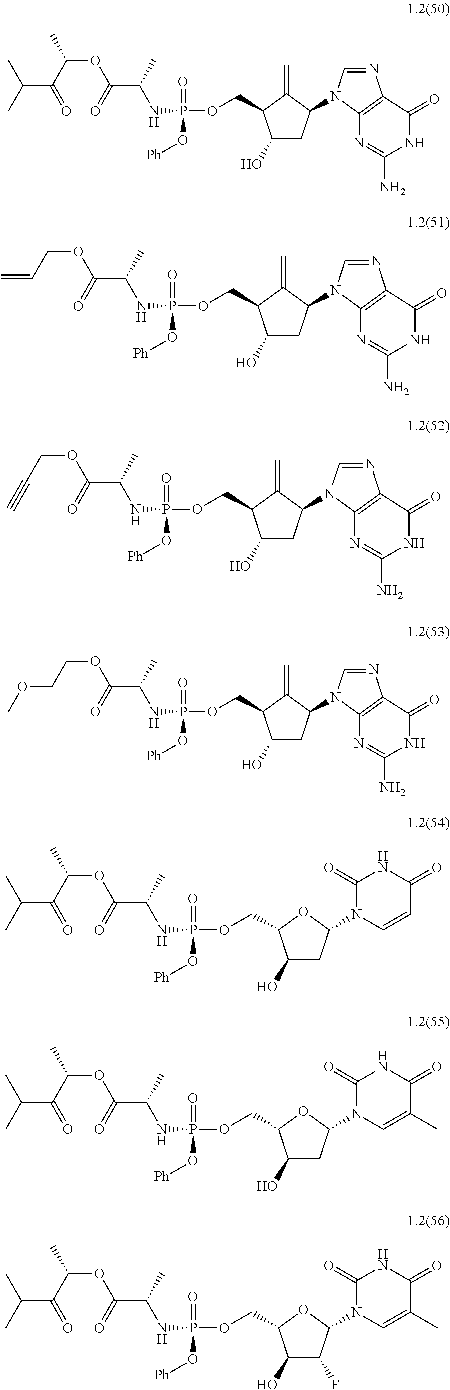

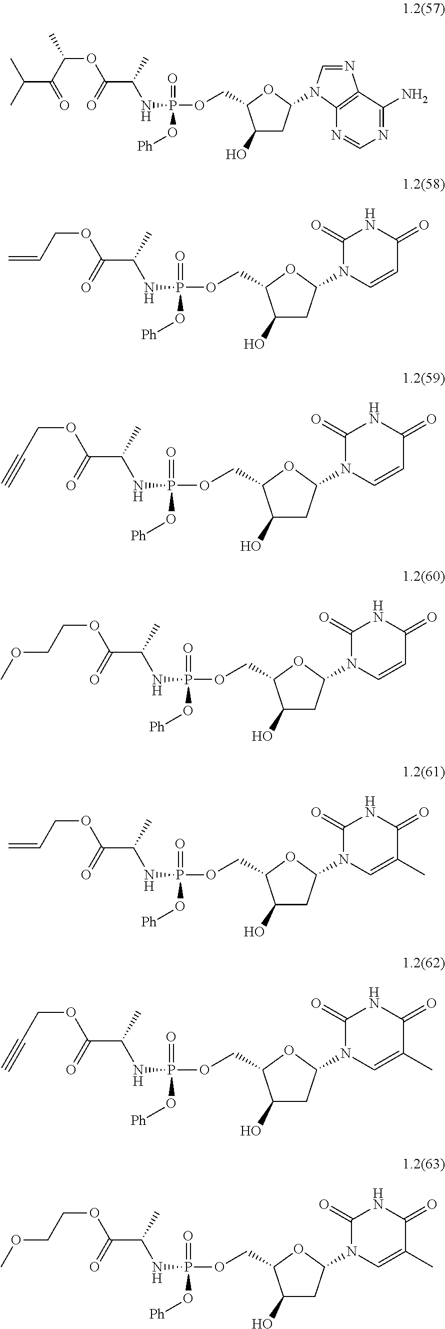

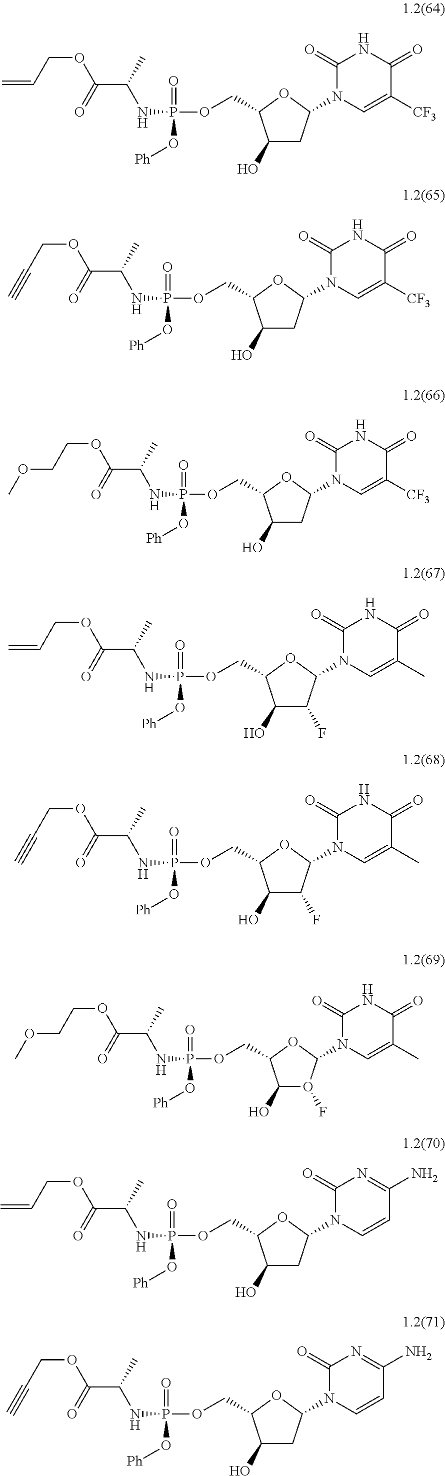

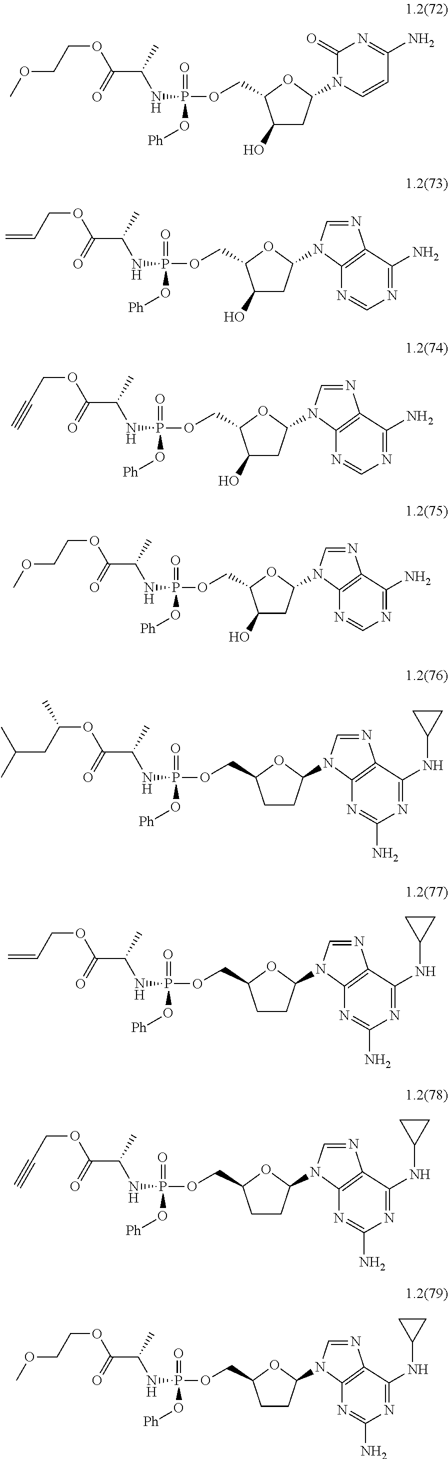

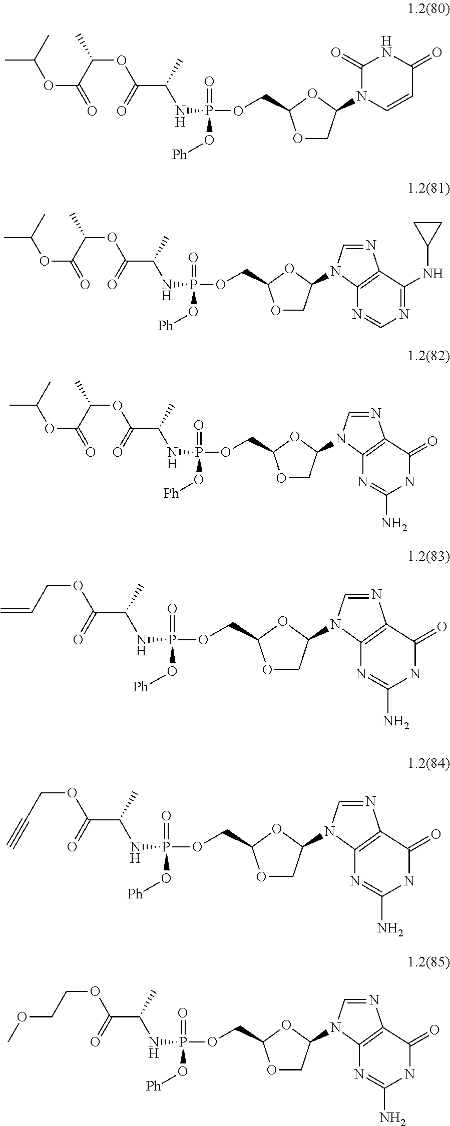

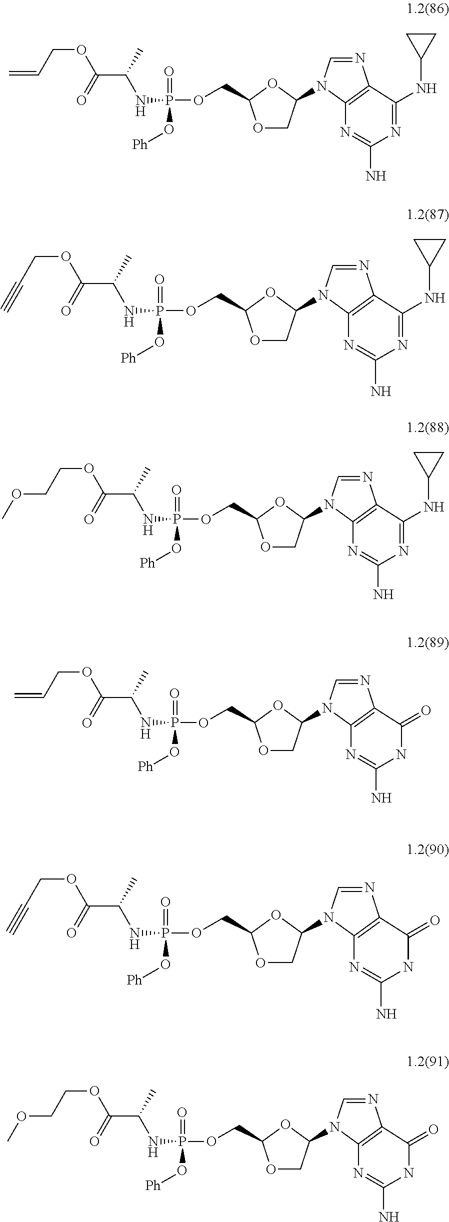

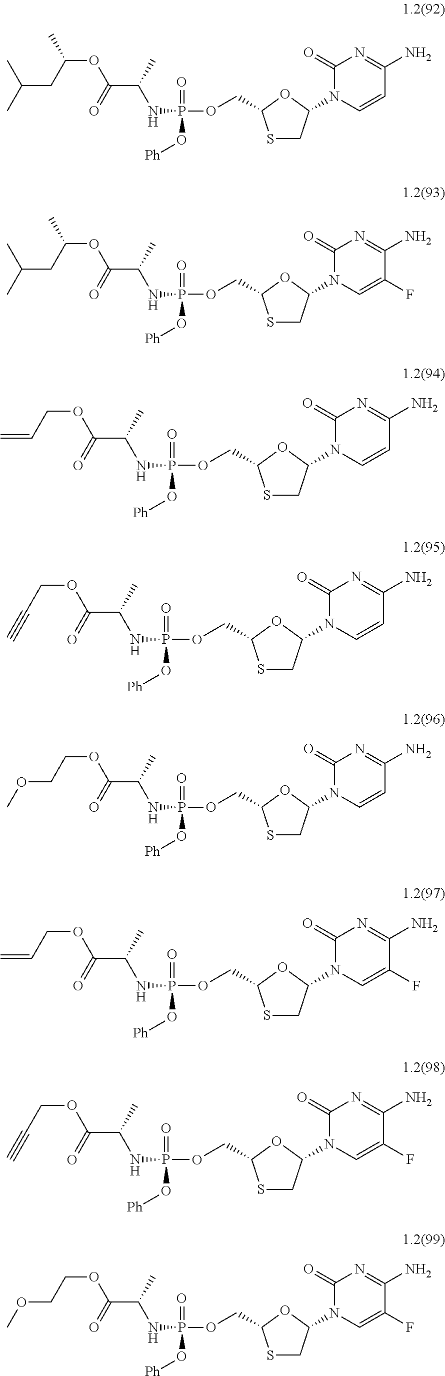

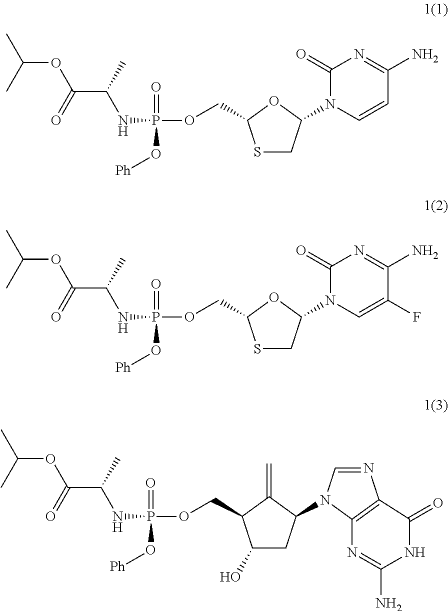 [SMILES: CC(C)OC(=O)[C@H](C)N[P@](=O)(OC[C@@H]1O[C@H](n2ccc(N)nc2=O)CS1)Oc1ccccc1 ; CC(C)OC(=O)[C@H](C)N[P@](=O)(OC[C@@H]1O[C@H](n2cc(F)c(N)nc2=O)CS1)Oc1ccccc1 ; C=C1[C@H](CO[P@@](=O)(N[C@@H](C)C(=O)OC(C)C)Oc2ccccc2)[C@@H](O)C[C@@H]1n1cnc2c(=O)[nH]c(N)nc21]

### Dosage, Administration, and Use

The subject of the present invention is a pharmaceutical composition comprising one or more of compounds of general formula 1, or stereoisomers, isotope-enriched analogues, pharmaceutically acceptable salts, hydrates, solvates, or crystalline or polymorphic forms thereof, optionally in combination with a pharmaceutically acceptable excipient, carrier, additive, diluent, and equivalent medium for the treatment of viral infections and/or neoplastic diseases in mammals.

The compounds of general formula 1 may be formulated in a wide variety of oral administration dosage forms and carriers, oral administration can be in the form of tablets, coated tablets, hard and soft gelatin capsules, solutions, emulsions, syrups, or suspensions. Compounds of the present invention are efficacious when administered by suppository administration, among other routes of administration. The most convenient manner of administration is generally oral using a convenient daily dosing regimen which can be adjusted according to the severity of the disease and the patient's response to the antiviral and anticancer medication.

A phosphoramidate nucleoside prodrug of general formula 1, its stereoisomers, isotope-enriched analogues, pharmaceutically acceptable salts, hydrates, solvates, or crystalline or polymorphic forms thereof together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as suspensions, emulsions, or filled capsules for oral use; or in the form of suppositories for rectal or vaginal administration. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the desired dose and pharmacokinetic parameters.

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

Solid form preparations include powders, tablets, pills, capsules, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs and aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous suspensions can be prepared by dispersing the finely divided active component in water with viscous materials such as natural or synthetic gums, resins, methylcellulose, sodium carboxymethylcellulose, and other well known suspending agents.

The phosphoramidate nucleoside prodrug of general formula 1, its stereoisomers, isotope-enriched analogues, pharmaceutically acceptable salts, hydrates, solvates, or crystalline or polymorphic forms thereof, may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The phosphoramidate nucleoside prodrug of general formula 1, its stereoisomers, isotope-enriched analogues, pharmaceutically acceptable salts, hydrates, solvates, or crystalline or polymorphic forms thereof, may be formulated for vaginal administration. Pessaries, tampons, creams, gels, pastes, foams or sprays containing in addition to the active ingredient such carriers as are known in the art to be appropriate.

The subject of the invention is directed to a use of the phosphoramidate nucleoside prodrug of general formula 1, its stereoisomers, isotope-enriched analogues, pharmaceutically acceptable salts, hydrates, solvates, or crystalline or polymorphic forms thereof, in the manufacture of a medicament for the treatment of viral and cancerous diseases. It is contemplated that the compound represented by general formula 1, its stereoisomers, isotope-enriched analogues, pharmaceutically acceptable salts, hydrates, solvates, or crystalline or polymorphic forms thereof, in the manufacture of a medicament for the treatment of any of the antiviral and anticancer conditions disclosed herein can be any of the compounds of formula (1.1) and (1.2), or (S)-2-{[(R)-1-methyl-2-(6-amino-purin-9-yl)-ethoxy]-phenoxy-phosphorylamino}-propanoate (1.1(1)), 2-methoxy-ethyl (S)-2-{[(R)-1-methyl-2-(6-amino-purin-9-yl)-ethoxy]-phenoxy-phosphorylamino}-propanoate (1.1(2)), (S)-1-ethoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(1)), (S)-1-isopropyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(2)), (S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)), (S)-1-ethoxycarbonyl-cyclopropyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(4)), isopropyl (S)-2-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionylamino)-propanoate (1.2(5)), (S)-1-carboxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionate (1.2(6)), (S)-1-carbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(7)), (S)-1-dimethylcarbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(8)), allyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(9)), prop-2-ynyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(10)), 2-methoxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(11)), (S)-1-isopropoxycarbonyl-ethyl (S)-2-{(R)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(12)), (S)-1-isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13)), benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), 4-((S)-2-[(2R,3R,4R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butyric acid (1.2(15)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(16)), (S)-1-isopropoxycarbonyl-ethyl S)-2-{(S)-[(2R,3S,5R)-5-(2,4-dioxo-5-methyl-3,4-dihydro-2H-pyrimidin-1-yl)-3-hydroxy-tetrahydro-furan-2-ylmethoxy]-phenoxy-phosphorylamino}-propanoate (1.2(17)), (S)-1-isopropoxycarbonyl-ethyl S)-2-{(S)-[(2R,3S,5R)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(18)), (S)-1-isopropoxycarbonyl-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(19)), (S)-1-isopropoxycarbonyl-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(20)), (S)-1-isopropoxycarbonyl-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(21)), (S)-1-isopropoxycarbonyl-ethyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(22)), allyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(23)), prop-2-ynyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(24)), 2-methoxymethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(25)), allyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(26)), (S)-1-isopropoxycarbonyl-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(27)), (S)-1-isopropoxycarbonyl-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(28)), (S)-1-isopropoxycarbonyl-ethyl (S)-2-{(S)-[(2R,3R,4R,5R)-5-(2,4-doxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(29)), prop-2-ynyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(30)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(31)), prop-2-ynyl ester (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(32), 2-methoxy-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(33), allyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(34), allyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(35), prop-2-ynyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(36)), 2-methoxy-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(37)), allyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(38)), prop-2-ynyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(39)), 2-methoxy-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(40)), allyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(41)), prop-2-ynyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(42)), 2-methoxy-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(43)), allyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(44)), prop-2-ynyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(45)), 2-methoxy-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(46)), allyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(47)), prop-2-ynyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(48)), 2-methoxy-ethyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(49)), (S)-1-isopropoxycarbonyl-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(50)), allyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(51)), prop-2-ynyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(52)), 2-methoxy-ethyl(S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(53)), (S)-1-isopropoxycarbonyl-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(54)), (S)-1-isopropoxycarbonyl-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(55)), (S)-1-isopropoxycarbonyl-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(56)), (S)-1-isopropoxycarbonyl-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(57)), allyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(58)), prop-2-ynyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(59)), 2-methoxymethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(60)), allyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (11.2(61))), prop-2-ynyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(62)), 2-methoxy-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(63)), allyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(64)), prop-2-ynyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(65)), 2-methoxy-ethyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(66)), allyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(67)), prop-2-ynyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(68)), 2-methoxy-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(69)), allyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(70)), prop-2-ynyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(71)), 2-methoxy-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(72)), allyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(73)), prop-2-ynyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(74)), 2-methoxy-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(75)), (S)-1-isopropoxycarbonyl-ethyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(76)), allyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(77)), prop-2-ynyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(78)), 2-methoxy-ethyl (S)-2-{(S)-[(1S,4R)-4-(2-amino-6-cyclopropylamino-purin-9-yl)-cyclopent-2-enylmethoxy]-phenoxy-phosphorylamino}-propanoate (1.2(79)), (S)-1-isopropoxycarbonyl-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(80)), (S)-1-isopropoxycarbonyl-ethyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(81)), (S)-1-isopropoxycarbonyl-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(82)), allyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(83)), prop-2-ynyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(84)), 2-methoxy-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(85)), allyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(86)), prop-2-ynyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(87)), 2-methoxy-ethyl (S)-2-{(S)-[(2R,4R)-4-(6-cyclopropylamino-purin-9-yl)-[1,3]dioxolan-2-ylmethoxy]-phenoxy-phosphorylamino}-propanoate (1.2(88)), allyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(89)), prop-2-ynyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(90)), 2-methoxy-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(91)), (S)-1-isopropoxycarbonyl-ethyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1.2(92)), (S)-1-isopropoxycarbonyl-ethyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1.2(93)), allyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(94)), prop-2-ynyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(95)), 2-methoxy-ethyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(96)), allyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(97)), prop-2-ynyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(98)), 2-methoxy-ethyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(99)), isopropyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1(1)), isopropyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1(2)), isopropyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1(3)), their stereoisomers, isotope-enriched analogues, pharmaceutically acceptable salts, hydrates, solvates, or crystalline or polymorphic forms thereof, either alone or in combination with another compound of the present invention. A medicament includes, but is not limited to, any one of the compositions contemplated of the present invention.

The term “medicament” means a substance used in a method of treatment and/or prophylaxis of a subject in need thereof, wherein the substance includes, but is not limited to, a composition, a formulation, a dosage form, and the like, comprising the phosphoramidate nucleoside prodrug of general formula 1.

The subject of the present invention is directed to a method of treatment and/or prophylaxis in a subject in need thereof, said method comprises administering a therapeutically effective amount of the phosphoramidate nucleoside prodrug represented by general formula 1, its stereoisomers, isotope-enriched analogues, pharmaceutically acceptable salts, hydrates, solvates, or crystalline or polymorphic forms thereof to the subject.

The subject of the present invention is also directed to a method of treatment and/or prophylaxis in a subject in need thereof, said method comprises administering a therapeutically effective of at least two or more different phosphoramidate nucleoside prodrugs of general formula 1, their stereoisomers, isotope-enriched analogues, pharmaceutically acceptable salts, hydrates, solvates, or crystalline or polymorphic forms thereof, falling within the scope of the compound represented by general formula 1 to the subject.

The subject of the present invention is also directed to a method of treatment and/or prophylaxis in a subject in need thereof, said method comprises alternatively or concurrently administering a therapeutically effective of at least two phosphoramidate nucleoside prodrugs of general formula 1, their stereoisomers, isotope-enriched analogues, pharmaceutically acceptable salts, hydrates, solvates, or crystalline or polymorphic forms thereof, to the subject.

The term “subject” means a mammal, which includes, but is not limited to cattle, pigs, sheep, chicken, turkey, buffalo, llama, ostrich, dogs, cats, and humans, preferably the subject is a human. It is contemplated that in the method of treating a subject thereof of the sixth embodiment can be any of the compounds contemplated in any of the aspects of the first, second, and third embodiments or those specifically recited in the tables above, either alone or in combination with another compound of the present invention.

The term “therapeutically effective amount” as used herein means an amount required to reduce symptoms of the disease in an individual. The dose will be adjusted to the individual requirements in each particular ease. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.1 and about 10 g, including all values in between, per day should be appropriate in monotherapy and/or in combination therapy. A preferred daily dosage is between about 0.1 and about 7 g per day, more preferred 0.2 and about 5.0 g per day. Generally, treatment is initiated with a large initial “loading dose” to rapidly reduce or eliminate the virus following by a decreasing the dose to a level sufficient to prevent resurgence of the infection. One of ordinary skill in treating diseases described herein will be able, without undue experimentation and in reliance on personal knowledge, experience and the disclosures of this application, to assertain a therapeutically effective amount of the compounds of the present invention for a given disease and patient.

The subject of the present invention is directed to a method of treatment and/or prophylaxis in a subject in need thereof, said method comprises administering to the subject a therapeutically effective amount of a compound represented by general formula 1, its stereoisomers, isotope-enriched analogues, pharmaceutically acceptable salts, hydrates, solvates, or crystalline or polymorphic forms thereof, and a therapeutically effective amount of another antiviral agent; wherein the administration is concurrent or alternative. It is understood that the time between alternative administration can range between 1-24 hours, which includes any sub-range.

Examples of “another antiviral agents” include, but are not limited to: HCV NS3 protease inhibitors Examples of “another antiviral agents” include, but are not limited to: HCV NS3 protease inhibitors, HCV NS4 inhibitors (see US US 20140296136, U.S. Pat. No. 8,987,195, U.S. Pat. No. 7,973,040, US 2012214783); HCV NS4 inhibitors (see EP 1497282); HCV NS3/NS4 inhibitors (EP 2364984); HCV NS5A inhibitors (see C. Wang et al. Hepatitis C virus RNA elimination and development of resistance in replicon cells treated with BMS-790052. Antimicrob. Agents Chemother. 2012, 56, 1350-1358. https://en.wikipedia.org/wiki/Daclatasvir; A. V. Ivachtchenko et al. Discovery of Novel Highly Potent Hepatitis C Virus NS5A Inhibitor (AV4025). J. Med. Chem. 2014, 57, 7716-7730; patent application U.S. Ser. No. 14/845,333); Toll-like receptor agonists (see WO 2015023958, WO 2012097012); and other inhibitors (see WO 2014106019, WO 2014033176, WO 2014033170, WO 2014033167, WO 2013006394, US 20090163545).

More preferred is a pharmaceutical composition, which together with the novel compounds by general formula 1, their stereoisomers, isotope-enriched analogues, pharmaceutically acceptable salts, hydrates, solvates, or crystalline or polymorphic forms thereof, further includes an antiviral or anticancer drug in therapeutically effective amounts.

More preferred is a pharmaceutical composition, which together with the novel compounds by general formula 1, their stereoisomers, isotope-enriched analogues, pharmaceutically acceptable salts, hydrates, solvates, or crystalline or polymorphic forms thereof, further comprises a therapeutically effective amount the HCV NS5A inhibitor the selected from the group of Daclatasvir (Daklinza, BMS790052) [C. Wang et al. 2012], AV-4025 [A. V. Ivachtchenko et al. 2014], AV-4067 and AV-4084 [patent application U.S. Ser. No. 14/845,333.], previously unknown pan-genotypic HCV NS5A ingibitors AVI-4056 and AVI-4058, which is also the subject of this invention.

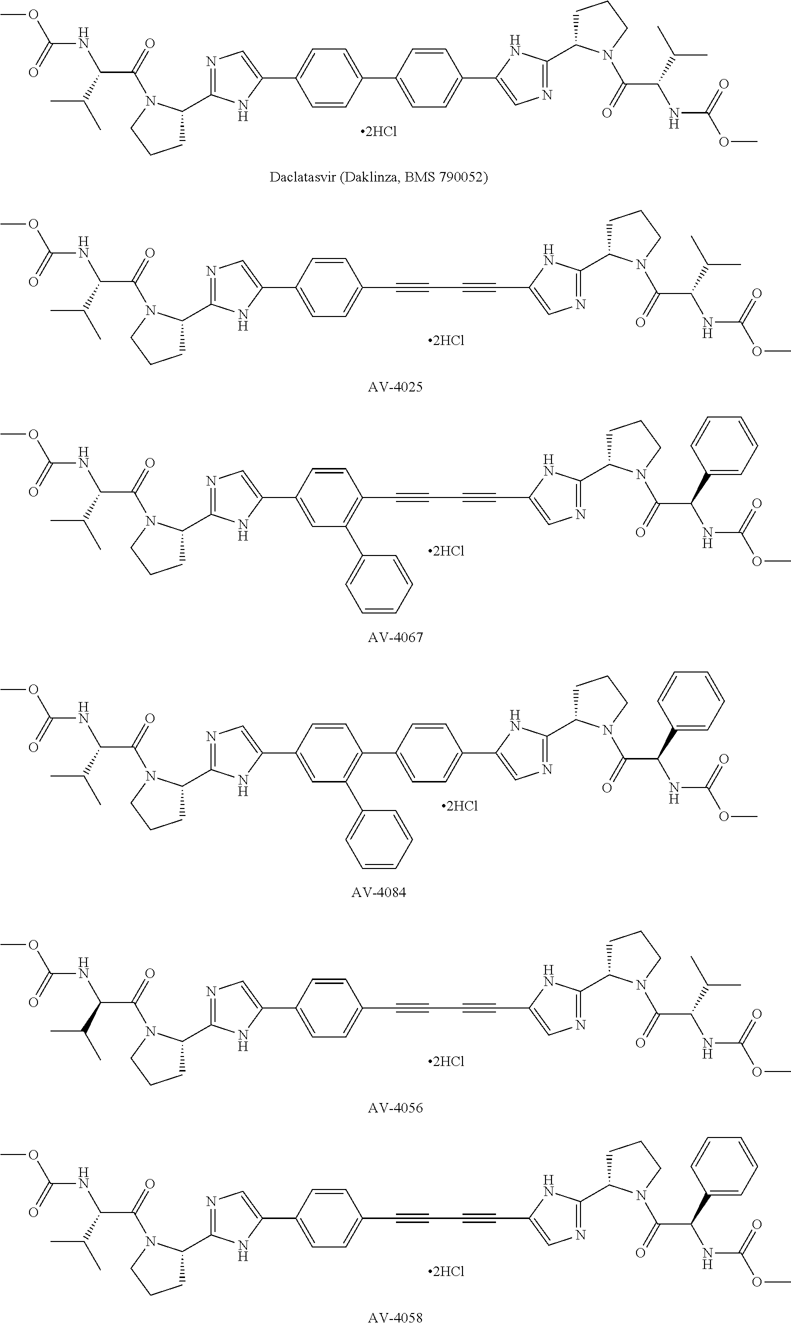

The subject of the present invention is directed to a method of treatment of viral and cancerous diseases in a subject in need thereof said method comprises alternatively or concurrently administering a therapeutically effective amount of a compound represented by general formula 1, its stereoisomers, isotope-enriched analogues, pharmaceutically acceptable salts, hydrates, solvates, or crystalline or polymorphic forms thereof, and another antiviral or anticancer agent to the subject. It is understood that the time between alternative administration can range between 1-24 hours, which includes any sub-range in between.

The subject of the present invention is directed to a method of treatment and/or prophylaxis in a subject in need thereof said method comprises administering to the subject a therapeutically effective of at least one compound represented by general formula 1, its stereoisomers, isotope-enriched analogues, pharmaceutically acceptable salts, hydrates, solvates, or crystalline or polymorphic forms thereof, and a therapeutically effective amount of another antiviral or anticancer agent; wherein the administration is concurrent or alternative. It is understood that the time between alternative administration can range between 1-24 hours, which includes any sub-range in between.

It is contemplated that the another antiviral agent includes, but is not limited to interferon-α, interferon-β, pegylated interferon-α, ribavirin, levovirin, viramidine, another nucleoside HCV polymerase inhibitor, a HCV non-nucleoside polymerase inhibitor, a HCV protease inhibitor, a HCV helicase inhibitor or a HCV fusion inhibitor, and a HBV DNA polymerase inhibitor and a HIV-1 reverse transcriptase (RT) inhibitor. When the active compound or its derivative or salt are administered in combination with another antiviral or anticancer agent the activity may be increased over the parent compound. When the treatment is combination therapy, such administration may be concurrent or sequential with respect to a compound represented by general formula 1 “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions. Furthermore, the term “treatment” of a viral infection, as used herein, also includes treatment or prophylaxis of a disease or a condition associated with or mediated by viral infection, or the clinical symptoms thereof.

### Process for Preparation

The present invention is a method for producing of the phosphoramidate nucleoside prodrug of general formula 1, its stereoisomers, isotope-enriched analogues, pharmaceutically acceptable salts, hydrates, solvates, or crystalline or polymorphic forms thereof, comprising the use of a compound of general formula 2 or a stereoisomer thereof and substituted alcohol of general formula 3 or stereoisomer thereof

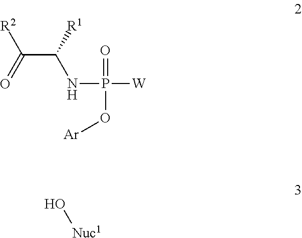

wherein: Ar, R¹ and R² have the above mentioned meaning; W is Cl or pentafluorophenyloxy; Nuc¹ is Nuc which has the above meaning and not necessarily having an O-protecting group or/and a N-protecting group.

The preferred reagents for producing of the phosphoramidate nucleoside prodrug of general formula 1, its stereoisomers, isotope-enriched analogues, pharmaceutically acceptable salts, hydrates, solvates, or crystalline or polymorphic forms thereof, are the compounds 2(1)-2(14)

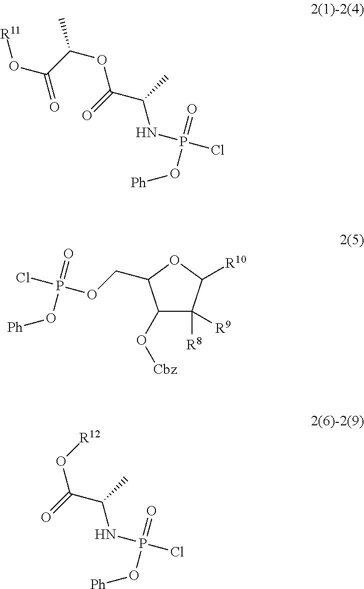

wherein: 2(1): R¹¹=Et. 2(2): R¹¹=i-Pr. 2(3): R¹¹═CH₂Ph. 2(4): R¹¹=c-Pr. 2(5): Cbz=protected group, X has the above mentioned meaning. 2(6): R¹²═CH₂—CH═CH₂. 2(7): R¹²═CH₂—C—CH. 2(8): R¹²═CH₂CH₂OCH₃. 2(9): R¹²═CH₂CH₂CH₂CO₂CH₂Ph

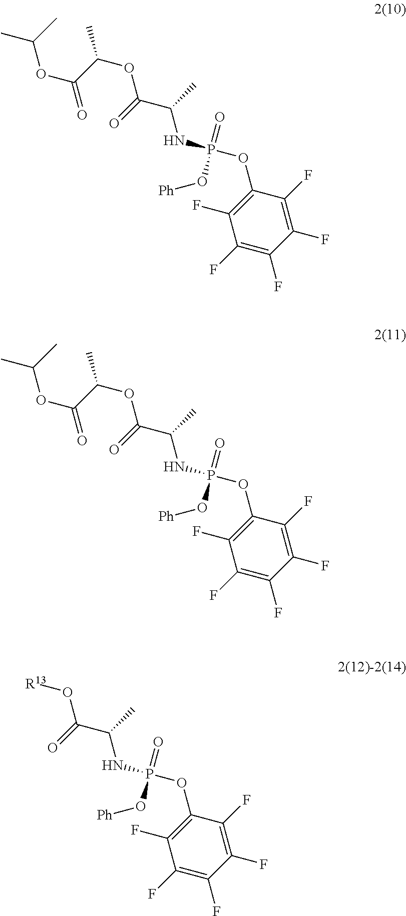

2(12): R¹³=allyl; 2(13): R¹³=prop-2-ynyl; 2(14): R¹³=2-methoxyethyl.

Synthesis of(S)-1-alkoxycarbonyl-ethyl (S)-2-(chloro-phenoxy-phosphorylamino)-propanoates 2(1)-2(4) was carried out according to the scheme 1:

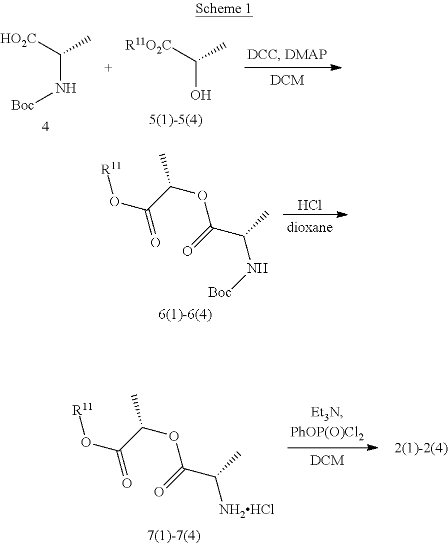

wherein: R¹¹ has the above mentioned meaning.

In accordance with Scheme 2 were obtained the reagents 2(5) and 5(7), which were used for the synthesis of the prodrugs of general formula 1.2(5)A, its stereoisomers, isotope-enriched analogues, pharmaceutically acceptable salts, hydrates, solvates, or crystalline or polymorphic forms thereof, including the prodrug 1.2(5):

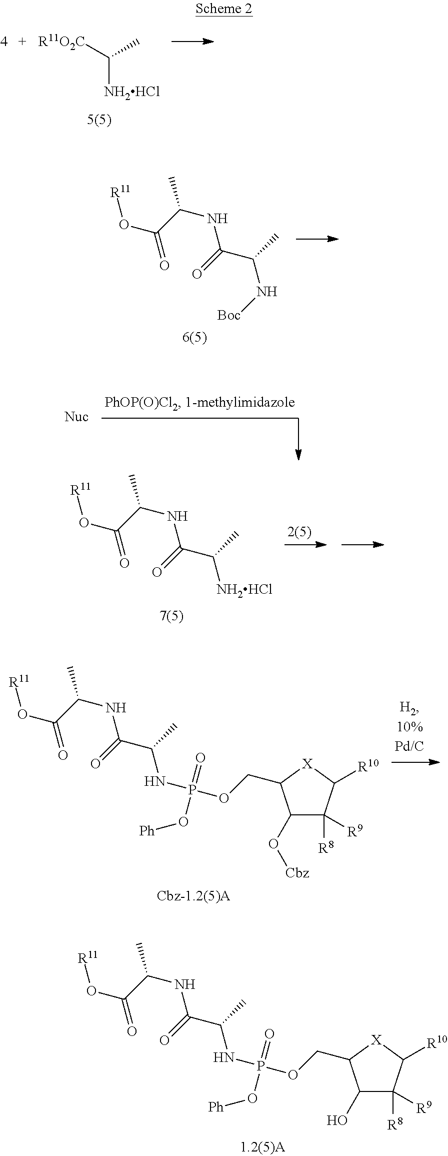

wherein: R⁸, R⁹, R¹⁰, R¹¹, and X have the above mentioned meaning.

Synthesis of the esters of (S)-2-(chloro-phenoxy-phosphorylamino)-propanoic acid 2(6)-2(9) were obtained by analogy with the synthesis of compounds 2(1)-2(4), described in example12, starting from (S)-2-(tert-butoxycarbonylamino)propanoic acid (4) and the corresponding alcohol 8 in accordance to the scheme 3:

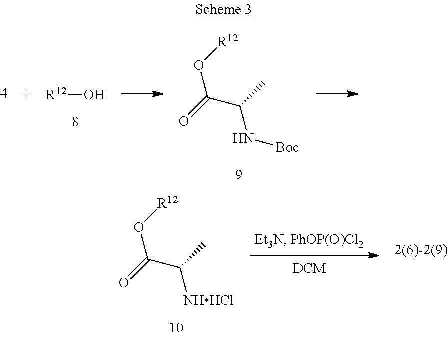

wherein: R¹² has the above mentioned meaning.

Synthesis of the reagents 2(10)-2(14) were prepared according to Scheme 4 starting from reagents 2(1)-2(4), 2(6)-2(9) and pentafluorophenol and followed by isolation of necessary phosphoric stereoisomers:

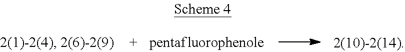

The subject of the invention is a process for preparing a compound of the general formula 1, its stereoisomers, isotope-enriched analogues, pharmaceutically acceptable salts, hydrates, solvates, or crystalline or polymorphic forms thereof, and its stereoisomer, wherein R¹═NR⁴R⁵, a Ar, R², R⁴, R⁵, X and Y are as defined above, reacting a compound of general formula 1 or its stereoisomer, in wherein R¹═OH, a Ar, R², X and Y are as defined above, or its activated derivative with an amine of general formula R⁴R⁵NH, wherein R⁴ and R⁵ are as defined above.

New phosphoramidite nucleoside prodrugs of general formula 1 are effective chemotherapeutic agents. For example, the prodrugs 1.2(2) and 1.2(9) are the more effective pan-genotypic inhibitors of HCV than the prototype PSI-7851 (Table 1.).

TABLE 1
Activity of the new pan-genotypic inhibitors HCV 1.2(2) and
1.2(9) and the prototype PSI-7851.

| | HCV Genotype | | | | | | |
|---|---|---|---|---|---|---|---|
| | gT1b | gT1b | | | | | |
| | 10% FBS | 40% NHS | gT1a | gT2a | gT3a | gT4a | gT5a |
| Prodrug | mean-min-max (IC₅₀, nM) | | | | | | |
| The | 61 | 80 | 103 | 39 | 152 | 350 | 395 |
| prototype | | | | | | | |
| PSI-7851 | | | | | | | |
| 1.2(2) | 18 | 34 | 57 | 24 | 69 | 80 | 185 |
| 1.2(9) | 29 | 34 | 54 | 23 | 51 | 100 | 146 |

Indeed inhibitory activity measured under comparable conditions of the new prodrugs and PSI-7851 indicates that the prodrugs 1.2(2) and 1.2(9) are 2-3 folders more effectively inhibit gT1b (10% FBS), than PSI-7851. ICgT1b50 these prodrugs has an average value (mean) 61 nM (for PSI-7851) 18 nM (for 1.2(2)) and 29 nM (for 1.2(9)).

The prodrugs of general formula 1 are also the highly effective inhibitors against HBV (Tabl 1b).

Test results of new inhibitors of the general formula 1 show their high activity and low cytotoxicity. Moreover, new prodrugs are surprisingly not only more potent than the prototype PSI-7851 (Table 1), but also have a more low cytotoxicity. Cell death at a concentration of 100 μM of prodrugs 1.2(2) and 1.2(9) is 14-16%, and at a concentration of 100 μM of the prototype PSI-7851 it is 31%.

The higher activity of the new prodrugs apparently related with a more efficient metabolism. Indeed prodrugs 1.2(2) and 1.2(9) are more efficiently metabolize into the key metabolite PSI-3 52707, which is a precursor to the 5′-mono- (PSI-7411), 5′-di- (PSI-7410) and 5′-triphosphate forms (PSI-7409), which are identical to the metabolites of the known prodrug PSI-7851. For example as compared to a PSI-7851 by the metabolism of prodrugs 1.2(2) and 1.2(9) in Huh7 cells is observed under comparable conditions a more efficient formation of the key metabolite PSI-3 52707 (see Table 2).

TABLE 2
Effectiveness of formation of the key metabolite
PSI-352707 by the metabolism of the prodrugs 1.2(2),
1.2(9) and prototype PSI-7851 in Huh7 cells.

| | Prodrugs | | |
|---|---|---|---|
| | 1.2(2) | 1.2(9) | The prototype PSI-7851 |
| Time, h | Concentration of PSI-352707, μM | | |
| 0.25 | 0.03 | 0.01 | 0.00 |
| 0.5 | 0.05 | 0.02 | 0.00 |
| 1 | 0.07 | 0.04 | 0.00 |
| 2 | 0.17 | 0.10 | 0.10 |
| 4 | 0.28 | 0.17 | 0.16 |
| 8 | 0.49 | 0.27 | 0.26 |

New phosphoramidite nucleoside prodrugs of general formula 1 are also effective chemotherapeutic agents against HBV (Table 3).

TABLE 3
Inhibitory activity and cytotoxicity of some new phosphoramidite
nucleoside prodrugs of general formula 1 against HBV.
Prodrug

| Prodrug | inhibition, % | viability, % | EC₅₀, μM | CC₅₀, μM |
|---|---|---|---|---|
| 1.2(50) | 100 | 95 | 0.04 ± 0.03 | >30 |
| 1.2(92) | 86 | 103 | 0.80 ± 0.61 | >30 |
| 1.2(93) | 93 | 94 | 0.04 ± 0.01 | >30 |
| 1.2(94) | 99 | 101 | 0.30 ± 0.09 | >30 |
| 1.2(96) | 96 | 89 | 0.32 ± 0.08 | >30 |
| 1.2(97) | 99 | 91 | 0.37 ± 0.05 | >30 |
| 1.2(67) | 88 | 95 | 0.76 ± 0.11 | >30 |

The present disclosure will now be described in connection with the certain embodiments, which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

### Examples

### Example 1. The General Procedure for the Synthesis of Phosphoramidate Nucleoside Prodrugs 1.1(1), 1.1(2)

To a solution of 0.42 mmol of (R)-1-(6-amino-9H-purin-9-yl)propan-2-ol (Nuc15) in 10 mL of THF was added tert-butyl magnesiumchloride 1M solution in THF (0.5 mL, 0.5 mmol, 1.2 eq) at 0° C. under Ar and the mixture was stirred for 0.5 h at room temperature. A solution of 0.5 mmol of the corresponding allyl ((S)-(perfluorophenoxy)(phenoxy)phosphorylamino)-propanoate 2(12) or 2-methoxy-ethyl ((S)-(perfluorophenoxy)(phenoxy)phosphorylamino)-propanoate (2(14)) 0.5 mmol in 2 mL of THF was added by syringe at 0-5° C. and reaction mixture was stirred for 15 h at room temperature under Ar. The reaction mixture was quenched with 0.5 mL of methanol and concentrated in vacuo, the residue was dissolved in DCM, washed with 5% citric acid, with brine, rotovapped and the desired prodrug 1.1(1) or 1.1(2) was separated by HPLC.

Alyl (S)-2-{[(R)-1-methyl-2-(6-amino-purin-9-yl)-ethoxy]-phenoxy-phosphorylamino}-propanoate (1.1(1)), LC-MS (ESI) 461 (M+H)⁺. ¹H NMR (DMSO-d₆, 400 MHz) δ 8.12 (s, 1H), 7.92 (s, 1H), 7.26 (t, J=8.0 Hz, 2H), 7.22 (s, 2H), 7.11 (t, J=7.2 Hz, 1H), 6.95 (d, J=8.4 Hz, 2H), 6.01 (dd, J₁=13.2 Hz, J₂=10.0 Hz, 1H), 5.89 (m, 1H), 5.32 (dd, J₁=17.2 Hz, J₂=1.2 Hz, 1H), 5.21 (dd, J₁=10.4 Hz, J₂=1.2 Hz, 1H), 4.87 (m, 1H), 4.56 (m, 2H), 4.30 (m, 2H), 3.75 (m, 1H), 1.27 (d, J=6.4 Hz, 3H), 1.15 (d, J=7.2 Hz, 3H).

2-Methoxy-ethyl (S)-2-{[(R)-1-methyl-2-(6-amino-purin-9-yl)-ethoxy]-phenoxy-phosphorylamino}-propanoate (1.1(2)), LC-MS (ESI) 479 (M+H)⁺. ¹H NMR (DMSO-d₆, 400 MHz) δ 8.12 (s, 1H), 7.92 (s, 1H), 7.26 (m, 2H), 7.19 (brs, 2H), 7.10 (m, 1H), 6.93 (m, 2H), 6.01 (dd, J₁=13.2 Hz, J₂=10.0 Hz, 1H), 4.85 (m, 1H), 4.29 (d, J=5.2 Hz, 2H), 4.16 (m, 2H), 3.71 (m, 1H), 3.52 (m, 2H), 3.25 (s, 3H), 1.27 (d, J=6.8 Hz, 3H), 1.13 (d, J=6.8 Hz, 3H).

### Example 2. The General Procedure for the Synthesis of Phosphoramidate Nucleoside Prodrugs 1.2(1)-1.2(3), 1.2(9)-1.2(11), 1.2(14)

To a mixture 0.75 mmol of the appropriate (chloro(phenoxy)phosphorylamino)-propanoate 2(1)-2(3), 2(6)-2(9) and 0.5 mmol of Nuc7, in 10 mL of acetonitrile was added 0.08 ml (1 mmol) N-methylimidazole and the mixture was stirred for 24 hours. At the end of the reaction (LC-MS control) the reaction mixture was concentrated in vacuo, the residue dissolved in dichloromethane, washed with 3% citric acid, brine, dried over sodium sulfate, evaporated and the desired prodrugs 1.2(1)-1.2(3), 1.2(9)-1.2(11), 1.2(14) were isolated by a suitable method.

(S)-1-Ethoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(1)), LC-MS (ESI) 588 (M+H)⁺; ¹H NMR (DMSO-d₆, 400 MHz) δ 11.52 (s, 1H), 7.55 (d, J=8.0 Hz, 1H), 7.38 (m, 2H), 7.20 (m, 3H), 6.18 (m, 1H), 6.03 (d, J=19.2 Hz, 1H), 5.87 (dd, J₁=24.0 Hz, J₂=7.2 Hz, 1H), 5.55 (m, 1H), 4.96 (m, 1H), 4.38 (m, 1H), 4.25 (m, 1H), 4.11 (m, 2H), 4.02 (m, 1H), 3.91 (m, 1H), 3.81 (m, 1H), 1.38 (dd, J₁=6.8 Hz, J₂=4.8 Hz, 3H), 1.26 (m, 6H), 1.17 (t, J=7.2 Hz, 3H).

(S)-1-(S)-1-Isopropyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino)}-propanoate (1.2(2)), LC-MS (ESI) 602 (M+H)⁺; ¹H NMR (DMSO-d₆, 400 MHz) δ 11.51 (s, 1H), 7.55 (d, J=7.6 Hz, 1H), 7.38 (m, 2H), 7.20 (m, 3H), 6.16 (m, 1H), 6.03 (d, J=20.0 Hz, 1H), 5.55 (m, 1H), 4.91 (m, 2H), 4.39 (m, 1H), 4.25 (m, 1H), 3.91 (m, 4H), 1.37 (m, 3H), 1.26 (m, 6H), 1.18 (dd, J₁=8.6 Hz, J₂=6.6 Hz, 6H).

(S)-1-Benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.3(3)), LC-MS (ESI) 650 (M+H)⁺; ¹H NMR (DMSO-d₆, 400 MHz) δ 11.52 (s, 1H), 7.54 (d, J=7.6 Hz, 1H), 7.36 (m, 7H), 7.20 (m, 3H), 6.17 (m, 1H), 6.03 (d, J=19.6 Hz, 1H), 5.86 (dd, J₁=24.0 Hz, J₂=6.8 Hz, 1H), 5.54 (t, J=8.0 Hz, 1H), 5.15 (m, 2H), 5.06 (m, 1H), 4.38 (m, 1H), 4.24 (m, 1H), 4.01 (m, 1H), 3.91 (m, 1H), 3.81 (m, 1H), 1.40 (dd, J₁=7.2 Hz, J₂=4.0 Hz, 3H), 1.24 (m, 6H).

Allyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(9)), LC-MS (ESI) 528 (M+H)⁺; ¹H NMR (DMSO-d₆, 400 MHz) δ 11.51 (s, 1H), 7.55 (m, 1H), 7.37 (m, 2H), 7.20 (m, 3H), 6.13 (q, J=12.0 Hz, 1H), 6.03 (d, J=19.2 Hz, 1H), 5.87 (m, 2H), 5.55 (t, J=8.0 Hz, 1H), 5.30 (d, J=19.2 Hz, 1H), 5.20 (dd, J₁=10.6 Hz, J₂=1.0 Hz, 1H), 4.54 (m, 2H), 4.39 (m, 1H), 4.26 (m, 1H), 4.03 (m, 1H), 3.88 (m, 1H), 3.81 (brs, 1H), 1.25 (m, 6H).

Prop-2-ynyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(10)), LC-MS (ESI) 526 (M+H)⁺.

2-Methoxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(11)), LC-MS (ESI) 546 (M+H)⁺; ¹H NMR (DMSO-d₆, 400 MHz) δ 11.52 (s, 1H), 7.55 (m, 1H), 7.37 (m, 2H), 7.21 (m, 3H), 6.07 (m, 2H), 5.87 (dd, J₁=24.0 Hz, J₂=5.6 Hz, 1H), 5.55 (dd, J₁=11.6 Hz, J₂=8.4 Hz, 1H), 4.39 (m, 1H), 4.25 (m, 1H), 4.13 (m, 2H), 4.03 (m, 1H), 3.84 (m, 2H), 3.49 (m, 2H), 3.24 (s, 3H), 1.24 (m, 6H).

Benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), LC-MS (ESI) 664 (M+H)⁺.

### Example 3

Isopropyl (S)-2-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionylamino)-propanoate (1.2(5)). To a solution of 946 mg (5 mmol) of (S)-2-(tert-butoxycarbonylamino)propanoic acid (4) and 0.824 mL (7.5 mmol) of 4-methylmorpholine in 20 mL of MeCN at −10-5° C. was added methyl chloroformate (0.398 mL, 5.15 mmol), the mixture was stirred for 10 min and 5.5 mmol of L-alanine isopropyl ester hydrochloride was added followed by 4-methylmorpholine (1.375 mL, 12.5 mmol) addition. The mixture was stirred for 15 h at room temperature then rotovapped, dissolved in DCM, washed with 5% citric acid solution, with water, dried over Na₂SO₄ and rotovapped to afford 1.5 g (99%) of (S)-isopropyl 2-((S)-2-(tert-butoxycarbonylamino)-propanamido)propanoate (6(5)a), ¹H NMR (DMSO-d₆, 400 MHz) δ 8.12 (d, J=7.2 Hz, 1H), 6.84 (d, J=7.6 Hz, 1H), 4.86 (m, 1H), 4.17 (p, J=6.8 Hz, 1H), 3.98 (p, J=7.6 Hz, 1H), 1.36 (s, 9H), 1.26 (d, J=7.2 Hz, 3H), 1.17 (d, J=7.6 Hz, 6H), 1.15 (d, J=7.6 Hz, 3H). The latter was converted in the conditions of the synthesis of compounds 7(1)-7(4) to (S)-isopropyl 2-((S)-2-aminopropanamido)propanoate hydrochloride (7(5)a), ¹H NMR (DMSO-d₆, 400 MHz) δ 8.86 (d, J=6.8 Hz, 1H), 8.27 (s, 3H), 4.88 (septet, J=7.2 Hz, 1H), 4.25 (p, J=7.2 Hz, 1H), 3.85 (q, J=7.2 Hz, 1H), 1.37 (d, J=7.2 Hz, 3H), 1.30 (d, J=7.2 Hz, 3H), 1.18 (dd, J₁=7.6 Hz, J₂=6.4 Hz, 6H). To a solution of compound Nuc7 (540 mg, 1.37 mmol) and phenyl dichlorophosphate (0.209 mL, 1.4 mmol) in 20 mL of DCM was added a solution of 1-methylimidazole (0.11 mL, 1.38 mmol) in 2 mL of DCM at −78° C. The stirred mixture was allowed to warm up to room temperature, compound 7(5)a (360 mg, 1.5 mmol) followed by 1-methylimidazole (0.24 mL, 3 mmol) in 2 mL of DCM addition. The mixture was stirred for 15 h, washed with 5% citric acid solution, with water, rotovapped and subjected to HPLC to afford 249 mg (25%) of Cbz-1.2(5). LC-MS (ESI) 735 (M+H)⁺. A solution of obtained Cbz-1.2(5) (248 mg, 0.338 mmol) in 20 ml of EtOAc was hydrogenated with 25 mg 10% Pd on carbon for 14 h at 1 atm of hydrogen. The mixture was filtered through Celite, rotovapped and dried in vacuo to obtain 197 mg (97%) of 1.2(5). LC-MS (ESI) 601 (M+H)⁺.

### Example 4

Synthesis of (S)-1-carboxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(6)). A solution of 520 mg (0.8 mmol) of (S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)) in 15 ml dioxane was stirred with 50 mg 10% Pd/C under a hydrogen atmosphere for 15 h. The resulting solution was filtered and evaporated in vacuo. The yield of 1.2(6) was quantitative. LC-MS (ESI) 560 (M+H)⁺; ¹H NMR (DMSO-d₆, 400 MHz) δ 13.02 (brs, 1H), 11.51 (s, 1H), 7.55 (d, J=7.2 Hz, 1H), 7.38 (m, 2H), 7.21 (m, 3H), 6.14 (m, 1H), 6.03 (m, 1H), 5.55 (m, 1H), 4.89 (q, J=6.9 Hz, 1H), 4.40 (m, 1H), 4.25 (m, 1H), 3.86 (m, 2H), 1.37 (dd, J₁=6.8 Hz, J₂=4.0 Hz, 3H), 1.26 (m, 6H).

### Example 5. The General Procedure for the Synthesis of Phosphoramidate Nucleoside Prodrugs (1.2(7), 1.2(8))

To a solution of 224 mg (0.4 mmol) of (S)-1-carboxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(6)) in 7.5 mL dioxane and 7.5 mL of DMF was added 0.4 mmol of the appropriate amine hydrochloride, 167 mg (0.44 mmol) HATU and 0.139 ml of diisopropylethylamine.

The mixture was stirred for 24 h, evaporated in vacuo to dryness and the desired product 1.2(7) and 1.2(8) was isolated by HPLC.

(S)-1-Carbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate ((1.2(7)), LC-MS (ESI) 559 (M+H)⁺.

(S)-1-Dimethylcarbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(8)), LC-MS (ESI) 587 (M+H)⁺; ¹H NMR (DMSO-d₆, 400 MHz) δ 11.52 (s, 1H), 7.55 (d, J=8.0 Hz, 1H), 7.38 (m, 2H), 7.20 (m, 3H), 6.07 (m, 2H), 5.87 (dd, J₁=18.4 Hz, J₂=6.0 Hz, 1H), 5.55 (dd, J₁=10.8 Hz, J₂=8.0 Hz, 1H), 5.32 (m, 1H), 4.39 (m, 1H), 4.26 (m, 1H), 4.03 (m, 1H), 3.85 (m, 2H), 2.98 (d, J=2.8 Hz, 3H), 2.81 (s, 3H), 1.26 (m, 9H).

### Example 6. The General Procedure for the Synthesis of Phosphoramidate Nucleoside Prodrugs 1.2(12), 1.2(16), 1.2(18), 1.2(29), 1.2(54), 1.2(50), 1.2(55), 1.2(63), 1.2(92), 1.2(93), 1.2(94), 1.2(97)

To a solution of the appropriate Nuc (0.42 mmol) in 10 mL of THF was added tert-butyl magnesiumchloride 1M solution in THF (0.92 mL, 0.92 mmol, 2.2 eq) at 0° C. under Ar and the mixture was stirred for 0.5 h at room temperature. A solution of appropriate substituted (S)-2-((S)-pentafluorophenyloxy-phenoxy-phosphorylamino)-propanoate (2(10)-2(14)) (0.5 mmol) in 2 mL of THF was added by syringe at 0-5° C. and reaction mixture was stirred for 15 h at room temperature under Ar. The reaction mixture was quenched with 0.5 mL of methanol and concentrated in vacuo, the residue was dissolved in DCM, washed with 5% citric acid, with brine, rotovapped and the desired prodrug 1.2(12), 1.2(16), 1.2(18), 1.2(29), 1.2(54), 1.2(50), 1.2(55), 1.2(63), 1.2(92), 1.2(93), 1.2(94), 1.2(97) was separated by HPLC.

(S)-1-Isopropoxycarbonyl-ethyl (S)-2-{(R)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate 1.2(12), LC-MS (ESI) 602 (M+H)⁺; ¹H NMR (DMSO-d₆, 400 MHz) δ 11.51 (s, 1H), 7.55 (d, J=8.0 Hz, 1H), 7.38 (m, 2H), 7.19 (m, 3H), 6.17 (dd, J₁=12.4 Hz, J₂=10.0 Hz, 1H), 6.03 (d, J=20.4 Hz, 1H), 5.89 (d, J=6.8 Hz, 1H), 5.56 (d, J=8.0 Hz, 1H), 4.91 (m, 2H), 4.42 (m, 1H), 4.27 (m, 1H), 4.04 (m, 1H), 3.90 (m, 1H), 3.80 (m, 1H), 1.37 (d, J=7.2 Hz, 3H), 1.27 (d, J=6.8 Hz, 3H), 1.24 (d, J=22.4 Hz, 3H), 1.18 (dd, J₁=9.2 Hz, J₂=6.4 Hz, 6H).

(S)-1-Isopropoxycarbonyl-ethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(16)), LC-MS (ESI) 570 (M+H)⁺; ¹H NMR (DMSO-d₆, 400 MHz) δ 11.32 (s, 1H), 7.59 (d, J=8.0 Hz, 1H), 7.37 (t, J=8.0 Hz, 2H), 7.20 (m, 3H), 6.15 (m, 2H), 5.53 (d, J=8.0 Hz, 1H), 5.40 (d, J=4.0 Hz, 1H), 4.91 (m, 2H), 4.22 (m, 1H), 4.16 (m, 1H), 4.08 (m, 1H), 3.93 (m, 2H), 2.08 (m, 2H), 1.37 (d, J=7.2 Hz, 3H), 1.29 (d, J=7.2 Hz, 3H), 1.18 (dd, J₁=8.8 Hz, J₂=6.4 Hz, 6H).

(S)-1-Isopropoxycarbonyl-ethyl S)-2-{(S)-[(2R,3S,5R)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(18)), LC-MS (ESI) 638 (M+H)⁺; ¹H NMR (DMSO-d₆, 400 MHz) δ 11.91 (s, 1H), 8.10 (s, 1H), 7.35 (m, 2H), 7.18 (m, 3H), 6.10 (dd, J₁=13.2 Hz, J₂=10.0 Hz, 1H), 6.06 (t, J=6.4 Hz, 1H), 5.40 (d, J=4.4 Hz, 1H), 4.90 (m, 2H), 4.21 (m, 2H), 4.07 (m, 1H), 4.01 (m, 1H), 3.89 (m, 1H), 2.19 (m, 2H), 1.36 (d, J=6.8 Hz, 3H), 1.27 (d, J=6.8 Hz, 3H), 1.17 (dd, J₁=8.8 Hz, J₂=6.4 Hz, 6H).

(S)-1-Isopropoxycarbonyl-ethyl (S)-2-{(S)-[(2R,3R,4R,5R)-5-(2,4-doxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(29)), LC-MS (ESI) 602 (M+H)⁺. ¹H NMR (DMSO-d₆, 400 MHz) δ 11.52 (s, 1H), 7.55 (d, J=8.0 Hz, 1H), 7.38 (m, 2H), 7.21 (m, 3H), 6.16 (dd, J₁=12.4 Hz, J₂=10.0 Hz, 1H), 6.03 (d, J=20.4 Hz, 1H), 5.84 (d, J=6.8 Hz, 1H), 5.54 (d, J=8.0 Hz, 1H), 4.91 (m, 2H), 4.35 (m, 1H), 4.22 (m, 1H), 3.99 (m, 2H), 3.84 (m, 1H), 1.36 (d, J=7.2 Hz, 3H), 1.29 (d, J=6.8 Hz, 3H), 1.26 (d, J=22.4 Hz, 3H), 1.18 (dd, J₁=9.2 Hz, J₂=6.4 Hz, 6H).

(S)-1-Isopropoxycarbonyl-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(54)), LC-MS (ESI) 570 (M+H)⁺; ¹H NMR (DMSO-d₆, 400 MHz) δ 11.33 (s, 1H), 7.55 (d, J=8.0 Hz, 1H), 7.36 (t, J=8.0 Hz, 2H), 7.22 (m, 3H), 6.16 (m, 2H), 5.59 (d, J=8.0 Hz, 1H), 5.41 (d, J=4.0 Hz, 1H), 4.92 (m, 2H), 4.19 (m, 1H), 4.13 (m, 2H), 3.93 (m, 2H), 2.07 (m, 1H), 1.96 (m, 1H), 1.36 (d, J=7.2 Hz, 3H), 1.30 (d, J=7.2 Hz, 3H), 1.17 (dd, J₁=8.8 Hz, J₂=6.4 Hz, 6H).

(S)-1-Isopropoxycarbonyl-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(50), LC-MS (ESI) 619 (M+H)⁺; ¹H NMR (DMSO-d₆, 400 MHz) δ 10.56 (s, 1H), 7.66 (s, 1H), 7.38 (m, 2H), 7.24 (m, 2H), 7.18 (m, 1H), 6.40 (s, 2H), 6.11 (dd, J₁=13.2 Hz, J₂=10.4 Hz, 1H), 5.38 (m, 1H), 5.16 (s, 1H), 4.93 (m, 2H), 4.61 (s, 1H), 4.24 (m, 1H), 4.20 (m, 1H), 4.08 (m, 1H), 3.95 (m, 1H), 2.74 (m, 1H), 2.30 (m, 1H), 2.07 (m, 1H), 1.38 (d, J=6.8 Hz, 3H), 1.31 (d, J=7.2 Hz, 3H), 1.18 (dd, J₁=9.2 Hz, J₂=6.4 Hz, 6H).

(S)-1-Isopropoxycarbonyl-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(55)), LC-MS (ESI) 584 (M+H)⁺; ¹H NMR (DMSO-d₆, 400 MHz) δ 11.31 (s, 1H), 7.45 (d, J=0.8 Hz, 1H), 7.36 (m, 2H), 7.21 (m, 2H), 7.17 (m, 1H), 6.18 (m, 2H), 5.40 (d, J=4.4 Hz, 1H), 4.23 (m, 1H), 4.15 (m, 2H), 3.91 (m, 2H), 2.02 (m, 2H), 1.76 (s, 3H), 1.35 (d, J=7.2 Hz, 3H), 1.30 (d, J=7.2 Hz, 3H), 1.17 (dd, J₁=9.2 Hz, J₂=6.4 Hz, 6H).

2-Methoxy-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(63)), LC-MS (ESI) 528 (M+H)⁺. ¹H NMR (DMSO-d₆, 400 MHz) δ 11.30 (s, 1H), 7.45 (d, J=0.8 Hz, 1H), 7.36 (m, 2H), 7.21 (m, 2H), 7.17 (m, 1H), 6.18 (dd, J₁=7.2 Hz, J₂=6.0 Hz, 1H), 6.11 (dd, J₁=13.2 Hz, J₂=10.0 Hz, 1H), 5.39 (d, J=4.4 Hz, 1H), 4.24 (m, 1H), 4.13 (m, 4H), 3.93 (m, 1H), 3.85 (m, 1H), 3.48 (t, J=4.4 Hz, 2H), 3.23 (s, 3H), 2.01 (m, 2H), 1.77 (s, 3H), 1.24 (d, J=7.2 Hz, 3H).

(S)-1-Isopropoxycarbonyl-ethyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1.2(92)), LC-MS (ESI) 571 (M+H)⁺.

(S)-1-Isopropoxycarbonyl-ethyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1.2(93)), LC-MS (ESI) 589 (M+H)⁺; ¹H NMR (DMSO-d₆, 400 MHz) δ 7.95 (d, J=7.2 Hz, 1H), 7.87 (brs, 1H), 7.63 (brs, 1H), 7.36 (m, 2H), 7.21 (m, 3H), 6.19 (m, 2H), 5.35 (m, 1H), 4.91 (m, 2H), 4.29 (m, 2H), 3.91 (m, 1H), 3.41 (m, 1H), 3.12 (m, 1H), 1.34 (d, J=6.8 Hz, 3H), 1.30 (d, J=6.8 Hz, 3H), 1.18 (dd, J₁=8.8 Hz, J₂=6.0 Hz, 6H), 0.94 (s, 3H).

Allyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(94)), LC-MS (ESI) 497 (M+H)⁺; ¹H NMR (DMSO-d₆, 400 MHz) δ 7.72 (d, J=7.6 Hz, 1H), 7.36 (m, 3H), 7.25 (brs, 1H), 7.22 (m, 2H), 7.18 (m, 1H), 6.23 (t, J=5.6 Hz, 1H), 6.16 (dd, J₁=13.2 Hz, J₂=10.0 Hz, 1H), 5.87 (m, 1H), 5.73 (d, J=7.6 Hz, 1H), 5.35 (t, J=4.4 Hz, 1H), 5.30 (dd, J₁=17.2 Hz, J₂=1.6 Hz, 1H), 5.20 (dd, J₁=10.4 Hz, J₂=1.2 Hz, 1H), 4.54 (m, 2H), 4.27 (m, 2H), 3.89 (m, 1H), 3.41 (dd, J₁=11.6 Hz, J₂=5.2 Hz, 1H), 3.05 (dd, J₁=11.6 Hz, J₂=6.0 Hz, 1H), 1.26 (d, J=6.8 Hz, 3H).

Allyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(97)), LC-MS (ESI) 515 (M+H)⁺. ¹H NMR (DMSO-d₆, 400 MHz) δ 7.95 (d, J=7.2 Hz, 1H), 7.87 (brs, 1H), 7.63 (brs, 1H), 7.35 (t, J=7.6 Hz, 2H), 7.21 (d, J=8.0 Hz, 2H), 7.17 (t, J=7.6 Hz, 1H), 6.17 (m, 2H), 5.86 (m, 1H), 5.35 (t, J=4.0 Hz, 1H), 5.29 (dd, J₁=17.2 Hz, J₂=1.2 Hz, 1H), 5.18 (dd, J₁=10.4 Hz, J₂=1.2 Hz, 1H), 4.53 (m, 2H), 4.29 (m, 2H), 3.88 (m, 1H), 3.41 (dd, J₁=11.6 Hz, J₂=5.2 Hz, 1H), 3.11 (dd, J₁=11.6 Hz, J₂=6.0 Hz, 1H), 1.25 (d, J=7.2 Hz, 3H).

### Example 7

(S)-1-Isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13)). To a solution 266 mg (0.5 mmol) of benzyl 1-((2R,4R,5R)-4-(benzyloxycarbonyloxy)-3,3-difluoro-5-(hydroxymethyl)-tetrahydrofuran-2-yl)-2-oxo-1,2-dihydropyrimidin-4-ylcarbamate (di-Cbz-Nuc18) [Pat. Appl. US 20150266918] in 10 mL of acetonitrile was added 0.75 mmol of (S)-((S)-1-isopropoxy-1-oxopropan-2-yl) 2-(chloro(phenoxy)phosphorylamino)propanoate (2(2)) and 0.08 mL (1 mmol) 1-methylimidazole and the mixture was stirred for 14 h at room temperature. The solvent was rotovapped, the residue was dissolved in DCM, washed with 5% citric acid solution, then with brine, dried over Na₂SO₄, rotovapped, and the product was separated by column chromatography on SiO₂ (PhMe:EtOAc from 1:1 to 1:2). Yield of di-Cbz-1(14) is 377 mg. LC-MS (ESI) 873 (M+H)⁺. The obtained di-Cbz-1.2(13) was hydrated on 50 mg 10% Pd/C in 10 mL of EtOAc and 3 mL of isopropanol, filtered through celite, rotovapped and purified by HPLC to afford 176 mg of prodrug 1.2(13).

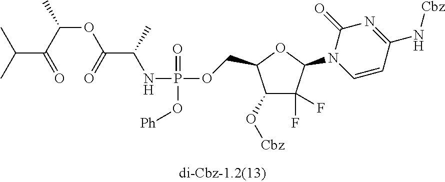 [SMILES: CC(C)C(=O)[C@H](C)OC(=O)[C@H](C)NP(=O)(OC[C@H]1O[C@@H](n2ccc(NC(=O)OCc3ccccc3)nc2=O)C(F)(F)[C@@H]1OC(=O)OCc1ccccc1)Oc1ccccc1]

(S)-1-Isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13), LC-MS (ESI) 605 (M+H)⁺. ¹H NMR (DMSO-d₆, 400 MHz) δ 7.46 (t, J=6.8 Hz, 1H), 7.38 (m, 4H), 7.20 (m, 3H), 6.41 (m, 1H), 6.18 (m, 2H), 5.74 (m, 1H), 4.24 (m, 3H), 4.02 (m, 1H), 3.92 (m, 1H), 1.36 (t, J=7.6 Hz, 3H), 1.29 (t, J=7.6 Hz, 3H), 1.17 (m, 6H).

### Example 8. The General Procedure for the Synthesis of Phosphoramidate Nucleoside Prodrugs 1.2(19), (1.2(40)

To a solution of benzyl 1-((2R,4R,5R)-4-(benzyloxycarbonyloxy)-3,3-difluoro-5-(hydroxymethyl)-tetrahydrofuran-2-yl)-2-oxo-1,2-dihydropyrimidin-4-ylcarbamate (di-Cbz-Nuc18) [US 20150266918] (300 mg, 0.56 mmol) in 20 mL of THF, tert-butylmagnesium chloride 1M solution in THF (0.70 mL, 0.62 mmol) was added at 0° C. in Ar atmosphere and the reaction mixture was stirred for 0.5 h at room temperature. The appropriate reagent 2(10) or (2(14) was added at 0-5° C. and reaction mixture was stirred for 14 h at room temperature in Ar atmosphere. The reaction mixture was diluted with methanol and concentrated in vacuo. The residue obtained after purification by column chromatography (silica gel, hexane:EtOAc, 1:1) gave respectively di-Cbz-1.2(19) or di-Cbz-1.2(40). Latest (0.4 mmol) in 15 ml of EtOAc and 10 ml of i-PrOH was hydrogenated with 22 mg of 10% Pd on carbon for 14 h at 1 atm of hydrogen. The resulting reaction mixture was filtered through Celite and evaporated. The residue was subjected to HPLC to afford of prodrug 1.2(19) or 1.2(40).

(S)-1-Isopropoxycarbonyl-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(19)), LC-MS (ESI) 605 (M+H)⁺; ¹H NMR (DMSO-d₆, 400 MHz) δ 7.45 (d, J=7.6 Hz, 1H), 7.39 (m, 4H), 7.21 (m, 3H), 6.39 (d, J=6.4 Hz, 1H), 6.18 (m, 2H), 5.74 (d, J=7.6 Hz, 1H), 4.30 (m, 1H), 4.19 (m, 2H), 3.99 (m, 1H), 3.93 (m, 1H), 1.35 (d, J=7.2 Hz, 3H), 1.30 (d, J=7.6 Hz, 3H), 1.18 (d, J₁=9.2 Hz, J₂=6.4 Hz, 6H).

2-Methoxyethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(40)), LC-MS (ESI) 549 (M+H)⁺. ¹H NMR (DMSO-d₆, 400 MHz) δ 7.45 (d, J=7.6 Hz, 1H), 7.42 (brs, 1H), 7.38 (m, 3H), 7.21 (m, 3H), 6.40 (d, J=6.4 Hz, 1H), 6.15 (m, 2H), 5.74 (d, J=7.6 Hz, 1H), 4.31 (m, 1H), 4.23 (m, 1H), 4.14 (m, 3H), 4.00 (m, 1H), 3.87 (m, 1H), 3.49 (t, J=4.8 Hz, 2H), 3.24 (s, 3H), 1.25 (d, J=7.2 Hz, 3H).

### Example 9. The General Procedure for the Synthesis of Phosphoramidate Nucleoside Prodrugs 1.2(21), 1.2(44), 1.2(46)

To a solution of 104 mg (0.35 mmol)2′-C-methyl-2′,3′-O-(1-methylethylidene)-cytidine (3(9)) [M. Donghi, B. Attenni, C. Gardelli et al. Synthesis and evaluation of novel phosphoramidate prodrugs of 2′-methyl cytidine as inhibitors of hepatitis c virus NS5B polymerase. Bioorg. Med. Chem. Lett. 2009, 19, 1392-1395.] in 10 mL of THF was added tert-butylmagnesium chloride 1M solution in THF (0.4 mL, 0.4 mmol) at 0° C. under Ar and the mixture was stirred for 0.5 h at room temperature. A solution of 0.4 mmol of the appropriate pentafluoroxy derivate 2(10), 2(12), 2(14) in 2 mL of THF was added by syringe at 0-5° C. and reaction mixture was stirred for 15 h at room temperature under Ar. The reaction mixture was quenched with 0.5 mL of methanol and concentrated in vacuo, the residue was dissolved in DCM, washed with 5% citric acid, with brine and rotovapped. The residue was dissolved in 10 mL of 80% aqueos TFA and stirred for 30 min, rotovapped and subjected to HPLC to afford of prodrugs 1.2(21), 1.2(44), 1.2(46).

(S)-1-Isopropoxycarbonyl-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(21)), LC-MS (ESI) 599 (M+H)⁺; ¹H NMR (DMSO-d₆, 400 MHz) δ 7.51 (d, J=7.2 Hz, 1H), 7.38 (m, 2H), 7.21 (m, 4H), 7.09 (brs, 1H), 6.12 (dd, J₁=12.4 Hz, J₂=11.2 Hz, 1H), 5.93 (s, 1H), 5.67 (d, J=7.2 Hz, 1H), 5.21 (d, J=7.2 Hz, 1H), 5.10 (s, 1H), 4.91 (m, 2H), 4.33 (m, 1H), 4.18 (m, 1H), 3.93 (m, 2H), 3.57 (m, 1H), 1.35 (d, J=6.8 Hz, 3H), 1.29 (d, J=6.8 Hz, 3H), 1.18 (dd, J₁=8.8 Hz, J₂=6.0 Hz, 6H), 0.94 (s, 3H).

Allyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(44)), LC-MS (ESI) 525 (M+H)⁺; ¹H NMR (DMSO-d₆, 400 MHz) δ 7.51 (d, J=7.6 Hz, 1H), 7.37 (m, 2H), 7.20 (m, 4H), 7.08 (brs, 1H), 6.09 (dd, J₁=12.4 Hz, J₂=10.4 Hz, 1H), 5.88 (m, 2H), 5.66 (d, J=7.6 Hz, 1H), 5.29 (m, 1H), 5.23 (d, J=7.2 Hz, 1H), 5.19 (m, 1H), 5.09 (s, 1H), 4.53 (m, 2H), 4.34 (m, 1H), 4.21 (m, 1H), 3.92 (m, 2H), 3.58 (m, 1H), 1.26 (d, J=7.2 Hz, 3H), 0.93 (s, 3H).

2-Methoxy-ethyl (S)-2-{(S)-[(2R,3R,4R,5R)-5-(4-amino-2-oxo-2H-pyrimidin-1-yl)-3,4-dihydroxy-4-methyl-tetrahydro-furan-2-ylmethoxy]-phenoxy-phosphorylamino}-propanoate (1.2(46)), LC-MS (ESI) 543 (M+H)⁺. ¹H NMR (DMSO-d₆, 400 MHz) δ 7.51 (d, J=7.2 Hz, 1H), 7.38 (m, 2H), 7.20 (m, 4H), 7.08 (brs, 1H), 6.07 (dd, J₁=12.8 Hz, J₂=10.4 Hz, 1H), 5.92 (brs, 1H), 5.66 (d, J=7.6 Hz, 1H), 5.22 (d, J=7.2 Hz, 1H), 5.09 (s, 1H), 4.34 (m, 1H), 4.21 (m, 1H), 4.12 (m, 2H), 3.94 (m, 1H), 3.89 (m, 1H), 3.58 (m, 1H), 3.48 (t, J=4.8 Hz, 2H), 3.24 (s, 3H), 1.24 (d, J=7.2 Hz, 3H), 0.93 (s, 3H).

### Example 10. The General Procedure for the Synthesis of Phosphoramidate Nucleoside Prodrugs 1.2(25), 1.2(33), 1.2(38), 1.2(39), 1.2(49), 1.2(57), 1.2(75)

To a solution of 0.4 mmol of the appropriate Cbz-Nu2, Nu7, Nu9, Nu23, Nu24, in 10 mL of THF was added tert-butylmagnesium chloride 1M solution in THF (0.5 mL, 0.5 mmol) at 0° C. under Ar and the mixture was stirred for 0.5 h at room temperature. A solution of 0.5 mmol of the appropriate perfluorophenoxy-derivative 2(11)-2(14) in 2 mL of THF was added by syringe at 0-5° C. and reaction mixture was stirred for 15 h at room temperature under Ar. The reaction mixture was quenched with 0.5 mL of methanol and concentrated in vacuo, the residue was dissolved in DCM, washed with 5% citric acid, with brine, rotovapped and the Cbz-prodrugs (Cbz-1.2(25), Cbz-1.2(33), Cbz-1.2(38), Cbz-1.2(39), Cbz-1.2(49), Cbz-1.2(57), Cbz-1.2(75)). was separated by column chromatography on silica gel (hexane:EtOAc=1:1). A solution of 0.315 mmol of the Cbz-prodrugs (Cbz-1.2(25), Cbz-1.2(33), Cbz-1.2(38), Cbz-1.2(39), Cbz-1.2(49), Cbz-1.2(57), Cbz-1.2(75)) in 15 ml of EtOAc and 4 ml of i-PrOH was hydrogenated with 22 mg 10% Pd on carbon for 14 h at 1 atm of hydrogen. The mixture was filtered through Celite, rotovapped, purified by HPLC, and obtained the prodrug 1.2(25), 1.2(33), 1.2(38), 1.2(39), 1.2(49), 1.2(57), 1.2(75).

2-Methoxyethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(25)), LC-MS (ESI) 514 (M+H)⁺; ¹H NMR (DMSO-d₆, 400 MHz) δ 11.32 (s, 1H), 7.59 (d, J=8.0 Hz, 1H), 7.37 (m, 2H), 7.22 (m, 2H), 7.18 (m, 1H), 6.16 (t, J=6.8 Hz, 1H), 6.08 (dd, J₁=12.0 Hz, J₂=11.2 Hz, 1H), 5.52 (d, J=8.0 Hz, 1H), 5.40 (d, J=3.6 Hz, 1H), 4.23 (m, 1H), 4.13 (m, 4H), 3.93 (m, 1H), 3.87 (m, 1H), 3.50 (t, J=4.0 Hz, 2H), 3.24 (s, 3H), 2.08 (m, 2H), 1.24 (d, J=7.2 Hz, 3H).

2-Methoxyethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(33)), LC-MS (ESI) 546 (M+H)⁺; ¹H NMR (DMSO-d₆, 400 MHz) δ 11.50 (s, 1H), 7.55 (d, J=8.0 Hz, 1H), 7.39 (m, 2H), 7.23 (m, 2H), 7.18 (m, 1H), 6.09 (dd, J₁=12.8 Hz, J₂=10.0 Hz, 1H), 6.02 (d, J=20.8 Hz, 1H), 5.83 (d, J=6.8 Hz, 1H), 5.41 (dd, J₁=8.0 Hz, J₂=1.6 Hz, 1H), 4.34 (m, 1H), 4.24 (m, 1H), 4.13 (m, 2H), 4.01 (m, 1H), 3.86 (m, 2H), 3.49 (t, J=4.8 Hz, 2H), 3.24 (s, 3H), 1.25 (d, J=22.4 Hz, 3H), 1.25 (d, J=7.2 Hz, 3H).

Allyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(38)), LC-MS (ESI) 531 (M+H)⁺. ¹H NMR (DMSO-d₆, 400 MHz) δ 7.45 (d, J=7.6 Hz, 1H), 7.38 (m, 4H), 7.20 (m, 3H), 6.39 (d, J=6.4 Hz, 1H), 6.16 (m, 2H), 5.87 (m, 1H), 5.74 (d, J=7.6 Hz, 1H), 5.30 (dd, J₁=17.2 Hz, J₂=1.6 Hz, 1H), 5.20 (dd, J₁=10.4 Hz, J₂=1.2 Hz, 1H), 4.54 (m, 2H), 4.31 (m, 1H), 4.20 (m, 2H), 4.00 (m, 1H), 3.91 (m, 1H), 1.26 (d, J=7.2 Hz, 3H).

Prop-2-ynyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(39)), LC-MS (ESI) 529 (M+H)⁺. ¹H NMR (DMSO-d₆, 400 MHz) δ 7.45 (d, J=7.6 Hz, 1H), 7.38 (m, 4H), 7.21 (m, 3H), 6.39 (d, J=6.8 Hz, 1H), 6.19 (m, 2H), 5.73 (d, J=7.6 Hz, 1H), 4.69 (m, 2H), 4.32 (m, 1H), 4.20 (m, 2H), 4.00 (m, 1H), 3.92 (m, 1H), 3.56 (t, J=2.4 Hz, 1H), 1.26 (d, J=7.2 Hz, 3H).

2-Methoxy-ethyl (S)-2-{(S)-[(2R,3S,5R)-5-(6-amino-purin-9-yl)-3-hydroxy-tetrahydro-furan-2-ylmethoxy]-phenoxy-phosphorylamino}-propanoate (1.2(49)), LC-MS (ESI) 537 (M+H)⁺; ¹H NMR (DMSO-d₆, 400 MHz) δ 8.28 (s, 1H), 8.14 (s, 1H), 7.34 (m, 2H), 7.26 (brs, 2H), 7.18 (m, 3H), 6.36 (t, J=6.8 Hz, 1H), 6.03 (dd, J₁=12.8 Hz, J₂=10.0 Hz, 1H), 5.46 (d, J=4.4 Hz, 1H), 4.46 (m, 1H), 4.22 (m, 1H), 4.07 (m, 4H), 3.84 (m, 1H), 3.45 (t, J=4.4 Hz, 2H), 3.21 (s, 3H), 2.76 (m, 1H), 2.31 (m, 1H), 1.20 (d, J=7.2 Hz, 3H).

(S)-1-Isopropoxycarbonyl-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(57)), LC-MS (ESI) 593 (M+H)⁺; ¹H NMR (DMSO-d₆, 400 MHz) δ 8.25 (s, 1H), 8.13 (s, 1H), 7.32 (m, 2H), 7.26 (brs, 2H), 7.17 (m, 3H), 6.36 (t, J=6.8 Hz, 1H), 6.11 (dd, J₁=13.2 Hz, J₂=10.0 Hz, 1H), 5.47 (d, J=4.4 Hz, 1H), 4.89 (m, 2H), 4.43 (m, 1H), 4.20 (m, 1H), 4.11 (m, 1H), 4.01 (m, 1H), 3.87 (m, 1H), 2.71 (m, 1H), 2.29 (m, 1H), 1.29 (d, J=6.8 Hz, 3H), 1.25 (d, J=7.2 Hz, 3H), 1.29 (dd, J₁=10.0 Hz, J₂=6.4 Hz, 6H).

2-Methoxy-ethyl (S)-2-{(S)-[(2S,3R,5S)-5-(6-amino-purin-9-yl)-3-hydroxy-tetrahydro-furan-2-ylmethoxy]-phenoxy-phosphorylamino}-propanoate (1.2(75)), LC-MS (ESI) 537 (M+H)⁺; ¹H NMR (DMSO-d₆, 400 MHz) δ 8.25 (s, 1H), 8.13 (s, 1H), 7.32 (m, 2H), 7.27 (brs, 2H), 7.17 (m, 3H), 6.36 (t, J=6.8 Hz, 1H), 6.05 (dd, J₁=12.8 Hz, J₂=10.0 Hz, 1H), 5.46 (d, J=4.0 Hz, 1H), 4.44 (m, 1H), 4.20 (m, 1H), 4.12 (m, 2H), 4.07 (m, 1H), 4.03 (m, 1H), 3.81 (m, 1H), 3.44 (m, 2H), 3.21 (s, 3H), 2.71 (m, 1H), 2.29 (m, 1H), 1.20 (d, J=7.2 Hz, 3H).

### Example 11. The General Procedure for the Synthesis of Phosphoramidate Nucleoside Prodrugs 1.2(32), 1.2(34)

To a solution of Boc-Nuc7 (0.5 mmol) in 10 mL of THF was added tert-butylmagnesium chloride 1M solution in THF (1.1 mL, 1.1 mmol) at 0° C. under Ar and the mixture was stirred for 0.5 h at room temperature. A solution of the appropriate reagent 2(13) or 2(14) (0.6 mmol) in 2 mL of THF was added by syringe at 0-5° C. and reaction mixture was stirred for 15 h at room temperature under Ar. The reaction mixture was quenched with 0.5 mL of methanol and concentrated in vacuo, the residue was dissolved in DCM, washed with 5% citric acid solution, with brine, and rotovapped. The residue was dissolved in 4 mL of DCM, 4 mL of TFA was added and the mixture was stirred for 3 h, rotovapped, dissolved in DCM, washed with saturated NaHCO₃ solution, rotovapped and subjected to HPLC to give of the prodrug 1.2(32), 1.2(34).

Prop-2-ynyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(32), LC-MS (ESI) 526 (M+H)⁺. ¹H NMR (DMSO-d₆, 400 MHz) δ 11.51 (s, 1H), 7.55 (d, J=8.0 Hz, 1H), 7.38 (m, 2H), 7.23 (m, 2H), 7.19 (m, 1H), 6.17 (dd, J₁=12.4 Hz, J₂=10.0 Hz, 1H), 6.02 (d, J=19.2 Hz, 1H), 5.84 (d, J=6.8 Hz, 1H), 5.54 (d, J=8.0 Hz, 1H), 4.69 (m, 2H), 4.37 (m, 1H), 4.23 (m, 1H), 4.01 (m, 1H), 3.92 (m, 1H), 3.83 (m, 1H), 3.56 (t, J=2.4 Hz, 1H), 1.26 (d, J=7.2 Hz, 3H), 1.25 (d, J=22.4 Hz, 3H).

Allyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(34)), LC-MS (ESI) 528 (M+H)⁺. ¹H NMR (DMSO-d₆, 400 MHz) δ 11.51 (s, 1H), 7.56 (d, J=7.6 Hz, 1H), 7.36 (t, J=8.0 Hz, 2H), 7.22 (d, J=8.4 Hz, 2H), 7.18 (t, J=7.6 Hz, 1H), 6.12 (dd, J₁=12.4 Hz, J₂=10.0 Hz, 1H), 6.02 (m, 1H), 5.86 (m, 2H), 5.54 (d, J=7.6 Hz, 1H), 5.30 (dd, J₁=17.2 Hz, J₂=1.6 Hz, 1H), 5.20 (dd, J₁=10.4 Hz, J₂=1.2 Hz, 1H), 4.54 (m, 2H), 4.37 (m, 1H), 4.24 (m, 1H), 4.01 (m, 1H), 3.86 (m, 2H), 1.26 (m, 6H).

### Example 12. the General Procedure for the Synthesis of the (S)-1-alkoxycarbonyl-ethyl (S)-2-(chloro-phenoxy-phosphorylamino)-propanoates 2(1)-2(4)

To a solution of 19.3 g (0.102 mol) of (S)-2-(tert-butoxycarbonylamino)propanoic acid (4) and 0.1 mol of the corresponding L-lactate 5(1)-5(4) in 250 mL of DCM at 0-5° C. was added DCC (21.05 g, 0.102 mol) and DMAP (1.22 g, 10 mmol). The mixture was stirred for 4 h at 0-5° C., filtered, the precipitate was washed with ether, the filtrate was rotovapped and purified by column chromatography on silica gel (PhMe:EtOAc 19:1) to obtain of the corresponding ester of (S)-1-carboxy-ethyl (S)-2-tert-butoxycarbonylaminopropaionic acid 6(1)-6(4). (S)-1-Ethoxycarbonyl-ethyl (S)-2-tert-butoxycarbonylamino-propanoate (6(1)): ¹H NMR (DMSO-d₆, 400 MHz) δ 5.14 (q, J=7.2 Hz, 2H), 5.02 (bs, 1H), 4.39 (m, 1H), 4.10 (dq, J₁=7.2 Hz, J₂=0.8 Hz, 2H), 1.53 (d, J=7.2 Hz, 3H), 1.47 (d, J=7.2 Hz, 3H), 1.46 (s, 9H), 1.28 (t, J=7.2 Hz, 3H). (S)-1-Isopropoxycarbonyl-ethyl (S)-2-tert-butoxycarbonylamino-propanoate (6(2)): ¹H NMR (DMSO-d₆, 400 MHz) δ 7.31 (d, J=6.8 Hz, 1H), 4.93 (m, 2H), 4.05 (p, J=7.2 Hz, 1H), 1.39 (d, J=6.8 Hz, 3H), 1.37 (s, 8H), 1.34 (brs, 1H), 1.28 (d, J=7.6 Hz, 3H), 1.18 (dd, J₁=8.8 Hz, J₂=6.0 Hz, 6H). (S)-1-Benzyloxycarbonyl-ethyl (S)-2-tert-butoxycarbonylamino-propanoate (6(3)): ¹H NMR (DMSO-d₆, 400 MHz) δ 7.36 (m, 6H), 5.15 (s, 2H), 5.12 (m, 1H), 4.04 (m, 1H), 1.43 (d, J=7.2 Hz, 3H), 1.37 (s, 9H), 1.23 (t, J=7.2 Hz, 3H). To a solution of 98 mmol of the corresponding ester of (S)-1-carboxy-ethyl (S)-2-tert.-butoxycarbonylaminopropanoic acid 6(1)-6(4) in 200 mL of dioxane was added 200 mL of 3M HCl solution in dioxane. The mixture was stirred for 15 h, then evaporated to dryness and treated with 300 mL of ether. After crystallization the salt was grinded under ether, filtered off, washed with ether and dried in vacuum to obtain of ester of aminoacid 7(1)-7(4). (S)-1-Isopropoxycarbonyl-ethyl (S)-2-amino-propanoic acid hydrochloride (7(2)): ¹H NMR (DMSO-d₆, 400 MHz) δ 8.72 (s, 3H), 5.09 (q, J=6.8 Hz, 1H), 4.93 (septet, J=6.4 Hz, 1H), 4.12 (q, J=7.2 Hz, 1H), 1.48 (d, J=7.2 Hz, 3H), 1.44 (d, J=6.8 Hz, 3H), 1.20 (t, J=6.0 Hz, 6H). A solution of 8 mmol of the corresponding L-alanine ester hydrochloride 7(1)-7(4) and 1.195 ml (8 mmol) of phenyl dichlorophosphate in 30 ml of dichloromethane, cooled with dry ice and acetone to a temperature of <−70° C. was added dropwise 4.4 ml (16 mmol) of triethylamine, 2.166 g (10.5 mmol) DCC and 0.244 g (2 mmol) DMAP, stirred for 15 hours, filtered, washed with ether, the filtrate was evaporated and the residue was purified on silica gel (PhMe:EtOAc=9:1) to give the corresponding ester of (S)-2-[(S)-2-(chloro-phenoxy-phosphorylamino)-propanoylamino]-propanoate 2(1)-2(4).

(S)-1-Ethoxycarbonyl-ethyl (S)-2-chloro-phenoxy-phosphorylamino)-propanoate (2(1)): ¹H NMR (CDCl₃, 400 MHz) δ 7.39 (m, 2H), 7.27 (m, 3H), 5.18 (m, 1H), 4.26 (m, 4H), 1.61 (dd, J₁=6.4 Hz, J₂=4.8 Hz, 3H), 1.56 (dd, J₁=6.8 Hz, J₂=4.0 Hz, 3H), 1.29 (dt, J₁=7.2 Hz, J₂=2.8 Hz, 3H).

(S)-1-Isopropoxycarbonyl-ethyl (S)-2-chloro-phenoxy-phosphorylamino)-propanoate (2(2)): ¹H NMR (CDCl₃, 400 MHz) δ 7.39 (m, 2H), 7.28 (m, 3H), 5.14 (m, 1H), 5.07 (m, 1H), 4.27 (m, 2H), 1.61 (dd, J₁=6.4 Hz, J₂=4.8 Hz, 3H), 1.54 (dd, J₁=6.4 Hz, J₂=3.6 Hz, 3H), 1.27 (m, 6H).

(S)-1-Benzyloxycarbonyl-ethyl (S)-2-chloro-phenoxy-phosphorylamino)-propanoate (2(3)): ¹H NMR (CDCl₃, 400 MHz) δ 7.39 (m, 2H), 7.27 (m, 3H), 5.06 (septet, J=6.4 Hz, 1H), 4.29 (m, 1H), 2.61 (t, J=6.0 Hz, 2H), 1.27 (t, J=6.4 Hz, 6H).

### Example 13. the General Procedure for the Synthesis of the Esters of (S)-2-(chloro-phenoxy-phosphorylamino)-propanoic acid 2(6)-2(9)

(S)-2-(chloro-phenoxy-phosphorylamino)-propanoic acid 2(6)-2(9) were obtained by analogy with the synthesis of compounds 2(1)-2(4), described in example 12, starting from phenyl dichlorophosphate and the corresponding esters of (S)-2-amino-propionic acid 6(6)-6(9).

Prop-2-ynyl (S)-2-(chloro-phenoxy-phosphorylamino)-propanoate (2(7)): ¹H NMR (CDCl₃, 400 MHz) δ 7.40 (m, 2H), 7.28 (m, 3H), 4.79 (m, 2H), 4.24 (m, 2H), 2.52, 2.54 (2t, J=2.4 Hz, 1H), 1.56, 1.57 (2d, J=6.8 Hz, 3H)

2-Methoxy-ethyl (S)-2-(chloro-phenoxy-phosphorylamino)-propanoate (2(8)): ¹H NMR (CDCl₃, 400 MHz) δ 7.39 (m, 2H), 7.27 (m, 3H), 4.35 (m, 2H), 4.26 (m, 2H), 3.63 (m, 2H), 3.40 (d, J=4.8 Hz, 3H), 1.55 (m, 3H) was obtained starting from 2-methoxy-ethyl (S)-2-(tert-butoxycarbonylamino)propanoate: ¹H NMR (CDCl₃, 400 MHz) δ 5.07 (brs, 1H), 4.31 (m, 3H), 3.60 (t, J=4.8 Hz, 2H), 3.38 (s, 3H), 1.45 (s, 9H), 1.40 (d, J=7.2 Hz, 3H).

Benzyl 4-[(S)-2-(chloro-phenoxy-phosphorylamino)-propionyloxy]-butanoate (2(9)): ¹H NMR (CDCl₃, 400 MHz) δ 7.36 (m, 7H), 7.27 (m, 3H), 5.14, 5.15 (2s, 2H), 4.44 (m, 1H), 4.24 (m, 2H), 4.17 (m, 1H), 2.47 (m, 2H), 2.04 (m, 2H), 1.50, 1.51 (2d, J=7.2 Hz, 3H) was obtained starting from benzyl 4-((S)-2-(tert-butoxycarbonylamino)propanoyloxy)butanoate: ¹H NMR (DMSO-d₆, 400 MHz) δ 7.35 (m, 5H), 7.26 (d, J=7.6 Hz, 1H), 5.09 (s, 2H), 4.06 (m, 2H), 3.97 (m, 1H), 2.45 (t, J=7.6 Hz, 2H), 1.84 (m, 2H), 1.36 (s, 8H), 1.31 (brs, 1H), 1.22 (d, J=7.2 Hz, 3H).

### Example 14. The General Procedure for the Synthesis of the Esters (S)-2-(pentafluorophenyloxy-phenoxy-phosphorylamino)-propanoic acid 2(10)-2(14)

To a stirred suspension of 91 mmol of the corresponding L-alanine ester hydrochloride 6(6)-6(9) in 250 mL of anhydrous DCM was added phenyl dichlorophosphate (13.6 mL, 91 mmol), the mixture was cooled to −70° C. and a solution of triethylamine (24.7 mL, 182 mmol) in 75 mL of dichloromethane was added over 1 h. After 1 h of stirring at −70° C. the mixture was allowed to warm up to −20° C. and a solution of pentafluorophenol (16.75 g, 91 mmol) and triethylamine (12.3 mL, 91 mmol) in 150 mL of dichloromethane was added over 1 h. The mixture was stirred at 4° C. for 15 h, concentrated under reduced pressure, the residue was treated with 300 mL of benzene, the solution was filtered and concentrated again to a volume of about 50 mL. The residue was treated with 0.5 L of hexane. After 1 h the formed precipitate was filtered, washed with hexane and dried on air. Additional crystallization from hexane (1:50 ratio) afforded of reagent 2(10)-2(14). The mother liquor left from 2(11) was rotovapped, purified by column chromatography on silica gel (hexane:EtOAc from 20:1 to 5:1) and twice recrystallized from hexane (1:25 ratio) to obtain 3.4 g of 2(10).

(S)-1-Isopropoxycarbonyl-ethyl (S)-2-((R)-pentafluorophenyloxy-phenoxy-phosphorylamino)-propanoate (2(10)), LC-MS (ESI) 526 (M+H)⁺. ¹H NMR (DMSO-d₆, 400 MHz) δ 7.41 (m, 2H), 7.25 (m, 3H), 6.95 (dd, J₁=14.0 Hz, J₂=10.0 Hz, 1H), 4.93 (m, 2H), 4.07 (m, 1H), 1.37 (d, J=7.2 Hz, 3H), 1.36 (d, J=6.8 Hz, 3H), 1.18 (dd, J₁=7.6 Hz, J₂=6.4 Hz, 6H).

(S)-1-Isopropoxycarbonyl-ethyl (S)-2-((S)-pentafluorophenyloxy-phenoxy-phosphorylamino)-propanoate (2(11)): LC-MS (ESI) 526 (M+H)⁺. ¹H NMR (DMSO-d₆, 400 MHz) δ 7.41 (m, 2H), 7.24 (m, 3H), 6.95 (dd, J₁=14.0 Hz, J₂=10.0 Hz, 1H), 4.91 (m, 2H), 4.05 (m, 1H), 1.38 (d, J=7.2 Hz, 3H), 1.34 (d, J=7.2 Hz, 3H), 1.17 (dd, J₁=7.6 Hz, J₂=6.4 Hz, 6H).

2-Methoxyethyl (S)-2-((S)-pentafluorophenyloxy-phenoxy-phosphorylamino)-propanoate (2(12)): LC-MS (ESI) 470 (M+H)⁺. ¹H NMR (DMSO-d₆, 400 MHz) δ 7.41 (m, 2H), 7.24 (m, 3H), 6.90 (dd, J₁=14.0 Hz, J₂=10.0 Hz, 1H), 4.14 (m, 2H), 3.99 (m, 1H), 3.49 (m, 2H), 3.23 (s, 3H), 1.29 (d, J=7.2 Hz, 3H).

Allyl (S)-2-((S)-pentafluorophenyloxy-phenoxy-phosphorylamino)-propanoate (2(13)): LC-MS (ESI) 452 (M+H)⁺. ¹H NMR (DMSO-d₆, 400 MHz) δ 7.41 (m, 2H), 7.23 (m, 3H), 6.91 (dd, J₁=14.0 Hz, J₂=10.0 Hz, 1H), 5.86 (m, 1H), 5.28 (m, 1H), 5.19 (m, 1H), 4.54 (d, J=5.2 Hz, 2H), 4.03 (m, 1H), 1.30 (d, J=7.2 Hz, 3H).

Prop-2-ynyl (S)-2-((S)-pentafluorophenyloxy-phenoxy-phosphorylamino)-propanoate (2(14)): LC-MS (ESI) 450 (M+H)⁺. ¹H NMR (DMSO-d₆, 400 MHz) δ 7.41 (m, 2H), 7.24 (m, 3H), 6.96 (dd, J₁=14.0 Hz, J₂=10.0 Hz, 1H), 4.70 (d, J=2.8 Hz, 2H), 4.03 (m, 1H), 3.56 (t, J=2.8 Hz, 1H), 1.29 (d, J=7.2 Hz, 3H).

### Example 15. The General Procedure for the Synthesis of the Reagents Cbz-Nuc2, Cbz-Nuc7, Cbz-Nuc23, Cbz-Nuc24

To a solution of (TBDMS-Nuc: 5.17 mmol) and DMAP (1.263 g, 10.34 mmol) in 50 mL of DCM was added dropwise benzyl chloroformate (1.107 mL, 7.75 mmol) at 0-5° C. Then the reaction mixture was warmed to room temperature and stirred overnight, then washed with 5% citric acid solution and brine. After drying over Na₂SO₄ and rotovapping TBDMS-Cbz-Nuc was used for the next step without additional purification. Yield quantitative. Benzyl (2R,3R,4R,5R)-2-((tert-butyldimethylsilyloxy)methyl)-5-(2,4-dioxo-3,4-dihydropyrimidin-1 (2H)-yl)-4-fluoro-4-methyl-tetrahydrofuran-3-yl carbonate (TBDMS-Cbz-Nuc7), LC-MS (ESI) 509 (M+H)⁺. ¹H NMR (DMSO-d₆, 400 MHz) δ 11.56 (s, 1H), 7.82 (d, J=8.0 Hz, 1H), 7.39 (m, 5H), 6.04 (d, J=18.8 Hz, 1H), 5.62 (d, J=8.0 Hz, 1H), 5.21 (s, 2H), 5.14 (m, 1H), 4.18 (d, J=9.2 Hz, 1H), 4.18 (dd, J₁=12.0 Hz, J₂=2.4 Hz, 1H), 3.77 (dd, J₁=12.0 Hz, J₂=3.2 Hz, 1H), 1.33 (d, J=22.8 Hz, 3H), 0.86 (s, 9H), 0.08 (d, J=3.2 Hz, 6H). To a solution of TBDMS-Cbz-Nuc (5.15 mmol) in 50 mL of DCM was added Et₃N.3HF (4.21 mL, 25.75 mmol) and the mixture was stirred for 24 h. The mixture was washed with water, dried over Na₂SO₄, diluted with 50 ml of toluene and rotovapped to a volume of about 30 mL. The precipitated product 3(11) was filtered off, washed with hexane and dried. Yield of Cbz-Nuc over 85%.

Benzyl (2R,3S,5R)-5-(2,4-dioxo-3,4-dihydropyrimidin-1 (2H)-yl)-2-(hydroxymethyl)-tetrahydrofuran-3-yl carbonate (Cbz-Nuc2), LC-MS (ESI) 363 (M+H)⁺.

Benzyl (2R,3R,4R,5R)-5-(2,4-dioxo-3,4-dihydropyrimidin-1 (2H)-yl)-4-fluoro-2-(hydroxymethyl)-4-methyl-tetrahydrofuran-3-yl carbonate (Cbz-Nuc7), LC-MS (ESI) 395 (M+H)⁺. ¹H NMR (DMSO-d₆, 400 MHz) δ 11.53 (s, 1H), 7.94 (d, J=8.0 Hz, 1H), 7.40 (m, 5H), 6.05 (d, J=19.2 Hz, 1H), 5.72 (d, J=8.0 Hz, 1H), 5.36 (brs, 1H), 5.22 (m, 2H), 5.12 (dd, J₁=20.0 Hz, J₂=8.4 Hz, 1H), 4.12 (m, 1H), 3.80 (m, 1H), 3.61 (m, 1H), 1.33 (d, J=23.2 Hz, 3H).

(2S,3R,5S)-5-(6-Amino-9H-purin-9-yl)-2-(hydroxymethyl)tetrahydrofuran-3-yl benzyl carbonate (Nuc23), LC-MS (ESI) 386 (M+H)⁺).

(2R,3S,5S)-5-(6-Amino-9H-purin-9-yl)-2-(hydroxymethyl)tetrahydrofuran-3-yl benzyl carbonate (Nuc24), LC-MS (ESI) 386 (M+H)⁺).

### Example 16. The General Procedure for the Synthesis of the Reagents Boc-Nuc3, Boc-Nuc7

To a solution of TBDMS-Nuc (8.85 mmol) and Boc₂O (19.3 g, 88.5 mmol) in 180 mL of dioxane was added 180 mL of 1N KOH solution. The mixture was stirred at room temperature for 15 h, then diluted with water and extracted with DCM. The organic extract was washed with 5% citric acid solution and brine, dried over Na₂SO₄ and rotovapped. Column chromatography on silica gel (hexane:EtOAc 3:1, 1:1) to give the product (TBDMS-Boc-Nuc). tert-Butyl (2S,3S,4R,5S)-2-((tert-butyldimethylsilyloxy)methyl)-4-fluoro-5-(5-methyl-2,4-dioxo-3,4-dihydropyrimidin-1 (2H)-yl)-tetrahydrofuran-3-yl carbonate (TBDMS-Boc-Nuc3), LC-MS (ESI) 475 (M+H)⁺. ¹H NMR (DMSO-d₆, 400 MHz) δ 11.52 (s, 1H), 7.38 (m, 1H), 6.15 (dd, J₁=17.6 Hz, J₂=4.4 Hz, 1H), 5.40 (m, 0.5H), 5.27 (m, 0.5H), 5.17 (m, 1H), 4.05 (m, 1H), 3.87 (m, 2H), 1.77 (d, J=0.8 Hz, 3H), 1.45 (s, 9H), 0.89 (s, 9H), 0.09 (s, 6H). TBDMS-Boc-Nuc7), LC-MS (ESI) 475 (M+H)⁺. ¹H NMR (DMSO-d₆, 400 MHz) δ 11.57 (s, 1H), 7.85 (d, J=8.0 Hz, 1H), 6.05 (d, J=19.2 Hz, 1H), 5.61 (d, J=8.0 Hz, 1H), 5.08 (dd, J₁=22.4 Hz, J₂=5.6 Hz, 1H), 4.18 (d, J=9.2 Hz, 1H), 4.01 (d, J=12.0 Hz, 1H), 3.76 (dd, J₁=12.0 Hz, J₂=2.4 Hz, 1H), 1.45 (s, 9H), 1.31 (d, J=22.8 Hz, 3H), 0.89 (s, 9H), 0.08 (s, 6H). To a solution of TBDMS-Boc-Nuc (3.6 mmol) in 50 mL of DCM was added triethylamine (5 mL, 36 mmol) and Et₃N.3HF (2.92 mL, 18 mmol) and the mixture was stirred for 24 h. The mixture was washed with water, dried over Na₂SO₄, diluted with 50 ml of hexane and rotovapped to a volume of about 20 mL. The precipitated product was filtered off, washed with hexane and dried. Yield of Boc-Nuc over 95%.

tert-Butyl (2S,3S,4R,5S)-4-fluoro-2-(hydroxymethyl)-5-(5-methyl-2,4-dioxo-3,4-dihydropyrimidin-1(2H)-yl)-tetrahydrofuran-3-yl carbonate (Boc-Nuc3), LC-MS (ESI) 361 (M+H)⁺. ¹H NMR (DMSO-d₆, 400 MHz) δ 11.49 (s, 1H), 7.60 (s, 1H), 6.11 (dd, J₁=17.6 Hz, J₂=4.4 Hz, 1H), 5.38 (m, 0.5H), 5.25 (m, 0.5H), 5.20 (m, 1.5H), 5.13 (m, 0.5H), 4.00 (m, 1H), 3.72 (m, 1H), 3.63 (m, 1H), 1.79 (s, 3H), 1.45 (s, 9H).

tert-Butyl (2R,3R,4R,5R)-5-(2,4-dioxo-3,4-dihydropyrimidin-1 (2H)-yl)-4-fluoro-2-(hydroxymethyl)-4-methyl-tetrahydrofuran-3-yl carbonate (Boc-Nuc7), LC-MS (ESI) 361 (M+H)⁺. ¹H NMR (DMSO-d₆, 400 MHz) δ 11.52 (s, 1H), 7.96 (d, J=8.0 Hz, 1H), 6.04 (d, J=18.4 Hz, 1H), 5.72 (d, J=8.0 Hz, 1H), 5.38 (brs, 1H), 5.05 (dd, J₁=20.4 Hz, J₂=9.2 Hz, 1H), 4.09 (m, 1H), 3.79 (m, 1H), 3.59 (m, 1H), 1.45 (s, 9H), 1.31 (d, J=22.8 Hz, 3H).

### Example 17

Preparation of a pharmaceutical composition in the form of a tablet. Starch (1600 mg), ground lactose (1600 mg), talc (400 mg), and 1000 mg of chemotherapeutic agent 1.2(19), 1.2(26), 1.2(29), 1.2(32), 1.2(33), 1.2(67), or 1.2(93) were mixed together and pressed into a bar. The resulting bar was comminuted into granules and sifted through a sieve to collect granules of 14-16 mesh. The granules thus obtained were shaped into tablets of a suitable form weighing 300-600 mg each.

### Example 18

Preparation of a pharmaceutical composition in the form of capsules. Prodrug 1.2(19), 1.2(26), 1.2(29), 1.2(32), 1.2(33), 1.2(67), or 1.2(93) and lactose powder were carefully mixed in the ratio 2:1. The resultant powdery mixture was packed into gelatin capsules of a suitable size, 300-600 mg in each capsule.

### Example 19

Preparation of a pharmaceutical composition in the form of compositions for intramuscular, intraperitoneal, or hypodermic injections. Prodrug 1.2(19), 1.2(26), 1.2(29), 1.2(32), 1.2(33), 1.2(67), or 1.2(93) (500 mg), chlorobutanol (300 mg), propylene glycol (2 ml), and injectable water (100 ml) were mixed together. The resultant solution was filtered, placed into 5 ml ampoules, and sealed.

### Example 20

Preparation of a pharmaceutical composition in the form of capsules. Prodrug 1.2(19), 1.2(26), 1.2(29), 1.2(32), 1.2(33), 1.2(67), or 1.2(93), the HCV NSSA inhibitor (Declatasvir, AV-4025, AV-4056, AV-4058, AV-4067, or AV-4084) and lactose powder were carefully mixed in the ratio 2:0.6:1. The resultant powdery mixture was packed into gelatin capsules of a suitable size, 300-600 mg in each capsule.

### Example 21

Anti-HCV activity (EC₅₀) and cytotoxicity (CC₅₀) of prodrugs of general formula 1. The HCV replicon assay was used to determine the antiviral activity of chemotherapeutic agents of general formula 1 (test compounds). Sovaldi (PSI-7977) was used as the reference drug. The test cell line used in the HCV Replicon Assay was the human hepatoma cell line Huh7 incorporating the HCV replicons synthesized by an outside vendor. 96-well plates were seeded with cells at a density of 7.5×10³ cells per well in 50 μl of assay media. The compound stock solution was made up freshly in an assay medium (DMEM 1×, Cellgro; cat. #10-013-CV) as a 2× stock. A total of 11 serial 3-fold dilutions of test compounds were prepared from the 2× stock in the assay media ranging from 20 nM-0.2 pM final concentrations. At least 4 hours after seeding the cells, compound treatment was initiated by adding 50 μl of compound dilution to the plates. The final concentrations of compound therefore ranged from 10 nM to 0.1 pM when diluted 1:1 in culture media. The final DMSO concentration was 0.5%. Cells and inhibitors were incubated for 3 days at 37° C./5% CO₂. The media was removed from the plates by gentle tapping. The cells were fixed with 100 μl 1:1 acetone:methanol for 1 minute, washed three times with PBS buffer, and then blocked with 150 μl/well 10% Fetal Bovine Serum (FBS) in PBS for 1 hour at room temperature. The cells were then washed three times with PBS buffer and incubated with 100 μl/well anti-hepatitis C core mAb (Affinity BioReagents; cat. # MA1-080, 1 mg/ml stock diluted 1:4,000 in 10% FBS-PBS) for 2 hours at 37° C. Then, the cells were washed three times with PBS and incubated with 100 μl/well HRP-Goat Anti-Mouse antibody (diluted 1:3.500 in 10% FBS-PBS) for 1 hour at 37° C. The cells were then washed three times with PBS and developed with an OPD solution, 100 μl/well (1 OPD tablet+12 ml citrate/phosphate buffer+5 μl 30% H₂O₂ per plate), for 30 minutes in the dark at room temperature. The reaction was stopped with 2N H₂SO₄ (100 μl/well), and the absorbance was measured at A₄₉₀ X on a Victor³ V 1420 multilabel counter (Perkin Elmer). The EC₅₀ values were calculated for test compounds from the resulting best-fit equations determined by Xlfit software (Table 1).

The cytotoxicity of the test compounds was studied in parallel using the same cell line, Huh7. Cell viability was determined using the ATPLite Kit (Perkin-Elmer, Boston, USA), according to manufacturer's instructions. 96-well black/transparent bottom plates were seeded with cells at a density of 7.5×10³ cells per well in 50 μl medium. After 18 hours, compound treatment was initiated by adding 50 μl of compound dilution into the plates. Each compound dilution was tested in triplicates. The cells and inhibitors were then incubated for 96 hours at 37° C./5% CO₂. The plates were washed twice with PBS (0.2 ml/well), and then lysed by adding lysis buffer, 0.05 ml/well (all reagents were included with the ATPLite Kit). After rocking for 5 min on a rocking platform, substrate buffer was added (0.05 ml/well). After additional 5-min incubation, the plates were kept in dark for 10 min, and the luminescence was read using TopCount NXT (Packard, Perkin Elmer). CC₅₀ values for all test compounds were determined using XLfit 4.1 software.

### Example 22

Anti-HBV activity of prodrugs of general formula 1 determination in the AD-38 cell line using a real-time quanitative PCR. a) Cell cultures. HepG2 AD38 cell line, harboring an integrated over-the-length HBV genomic DNA (Lander S, et. al, Antimicrobal Agents and Chemotherapy, 1997, pg. 1715-1720) were provided by Dr. C. Seeger, Fox Chase Cancer Center, Philadelphia, Pa.). Cells were grown in complete media (DMEM/F12 with 2 mM L-Glutamine (Thermo Scientific, Cat #11320033), 10% Fetal Bovine Serum (ThermoFisher Scientific, Cat#), 1% Antibiotic-antimitotic solution (ThermoFisher Scientific, Cat#15240096), and 0.3 μg/ml tetracyclin (Sigma, Cat # T7660-5G). Cells were plated onto Corning Biocoat 96-well plates (Corning, Cat #356407) in 225 ul of complete media (without teracyclin), 20000 cells/well. The prodrugs of general formula 1 were dissolved first in DMSO (Sigma cat. D2650), then in DMEM/F12 and 9 serial 3× dilutions were prepared and added to cells in 22.5 μl. The final prodrugs concentrations ranged from 10 μM to 1 nM. Cells grown in the presence of tetracyclin were used as a positive control, as HBV replication in this cell line is turned off by thetracyclin. Cell plates were incubated for 4 days at 37 C, 5% CO2. b) HBV DNA isolation. After 4 days, viral DNA was isolated from culture supernatants using PureLink® Pro 96 Genomic DNA Purification Kit (ThermoFisher Scientific, Cat # K183104A), according to manufacturer's recommendations. Eluted DNA samples were stored at −20 C. c) Quantitative real-time PCR (qPCR). PCR was performed on the CFX96™ Real-Time System (Bio-Rad, Hercules, Calif.) using AmpliTaq Gold® DNA Polymerase (Applied Biosystems®).

### Reaction Mix:

| 10X reaction buffer | 2 | μl |
|---|---|---|
| 25 mM MgCl₂ | 2 | μl |
| dNTP mix (ATP, GTP, CTP - 2 mM, UTP - 4 mM) | 2 | μl |
| ROX, 50 μM (Fisher Scientific117545000 | 0.04 | μl |
| 20X primers/probe mix (primers 6 μM, probe 5 μM) | 1 | μl |
| Uracil-DNA Glycosylase (1 U/μL) | 0.15 | μl |
| AmpliTaq Gold ® DNA Polymerase (5 U/μl) | 0.1 | μl |
| Water | 12.11 (up to 19.4 μl) | |
| DNA | 0.6 | μl |

### Cycling Program:

| | | | | |
|---|---|---|---|---|
| 50° C. | 2 minutes | | | |
| 95° C. | 10 minutes | | {close oversize brace} | |
| 95° C. | 15 seconds | | | |
| 60° C. | 30 seconds | 40 cycles | | |
| 72° C. | 30 seconds + 1 second | - read on | | the last step of cycle |
| | per cycle | | | |

Primers and Probe were Ordered from IDT (San Diego, Calif.):

HBV_rcDNA-S_FAM
56FAM/ATCCTCAAC/ZEN/CACCAGCACGGGACCA/3IABkFQ/
HBV_rcDNA-S_R
GAGGGATACATAGAGGTTCCTTGA
HBV_rcDNA-S_F
GTTGCCCGTTTGTCCTCTAATTC

Ct values (cycle threshold) were obtained for each prodrug sample and normalized to untreated cells, according to formula E=(1/(1+100%))̂(Ct[studied prodrug sample]−Ct[K-]), where E is a normalized expression of studied gene, Ct[K-] and Ct[studied prodrug sample] are Ct values of untreated cells and studied prodrug sample. Inhibition curves were obtained and EC50 values were calculated using the Graph Prizm software (Table 3).

### Example 23

### Stability of Prodrugs of General Formula 1 in Huh7 4A Cells.

Stock solutions of test compounds of general formula 1 and PSI-352707 (10 mM) were prepared in DMSO (Panreac) and stored at −20° C. Calibrating samples of PSI-352707 (20, 10, 5, 2 and 1, 0.5, 0.2, 0.1 μM) were prepared by dilution stock solution with 70% methanol-water mixture (Optima LC/MS, Fischer Scientific) mixture (1/1, v/v). Huh7 4A cells were seeded in 12-well plate (5·10⁵ cells per well) and incubated for 48 hours at 37° C. in 5% CO2 atmosphere in 1 mL of growth media (DMEM containing 10% fetal bovine serum, 100 IU/ml penicillin/100 μg/mL streptomycin, essential and nonessential amino acids, sodium piruvate and L-glutamine solution (all PanEco)). Then cells were incubated with 10 NM test compounds in fresh medium for up to 48 h. At selected times, the medium was removed, centrifuged at 14 000 rpm for 5 min, diluted 10 times by 70% methanol and analyzed by HPLC-MS. Cell layer was twice washed by PBS, and suspended in 0.5 mL cold 70% methanol (with 20 mM EDTA) and incubated overnight at −20° C. The samples were centrifugated at 14 000 rpm for 5 min, and the supernatants were analyzed by HPLC-MS. LC-MS/MS analysis was performed on QTrap 5500 System (AB Sciex) combined with 1290 UPLC System (Agilent). Separation was achieved on BioBasic AX column (50×2.1 mm, 5μ, Thermo Scientific). Mobile phase contained 10 mM ammonium formate (Panreac) in water-acetonitrile mixture (7/3, v/v), pH 8.75. Elution was performed in isocratic mode (0.7 mL/min for 1 min). The injection volume was 1 μL. ABSciex QTrap 5500 Source (TurbolonSpray) was operated in negative ionization mode (Source temperature 650° C., Source gas 1 and Source gas 2-65 and 40 psi respectively). PSI-352707 and IS (tolbutamide) were detected in MRM mode by transitions with m/z 410 to 150 for PSI-352707 (collision energy −30 kV). Data analysis and quantitation (Table 2) was performed in Analyst 1.5.2 Software (AB Sciex).

1. The phosphoramidate nucleoside prodrug of the general formula 1, a stereoisomer, isotope-enriched analogue, pharmaceutically acceptable salt, hydrate, solvate, or crystalline or polymorphic form thereof, 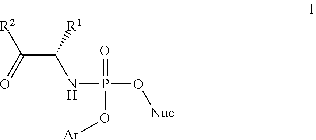
wherein:
Ar is aryl or hetaryl;
R¹ is H or CH₃;
R² is the substituent selected from OCH₂CH═CH₂, OCH₂C≡CH, OCH₂CH₂OCH₃, 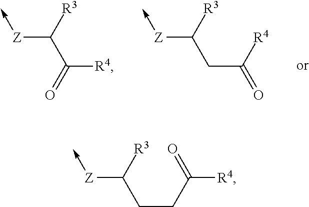
an arrow (→) indicates the place of substituent connection:
R³ is H or CH₃;
R⁴ is OH, OR⁵, NR⁶R⁷;
R⁵ is C₁-C₄-alkyl;
R⁶ and R⁷ are not necessarily the same substituents selected from H or CH₃;
Z═O, or NH;
Nuc is 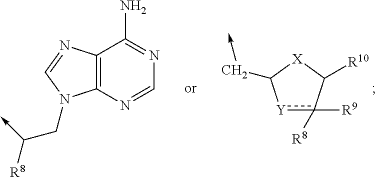
an arrow (→) indicates the place of substituent connection;
R⁸ and R⁹ are not necessarily the same substituents selected from H, F, Cl, CH₃ or OH provided when continuous line and its accompanying dotted line ( [symbol] ) together are the single carbon-carbon (C—C) bond
or
R⁸ and R⁹ are hydrogen provided when continuous line and its accompanying dotted line ( [symbol] ) together are the double carbon-carbon bond (C═C);
R¹⁰ is the substituent selected from R^{10.1}-R^{10.5}; 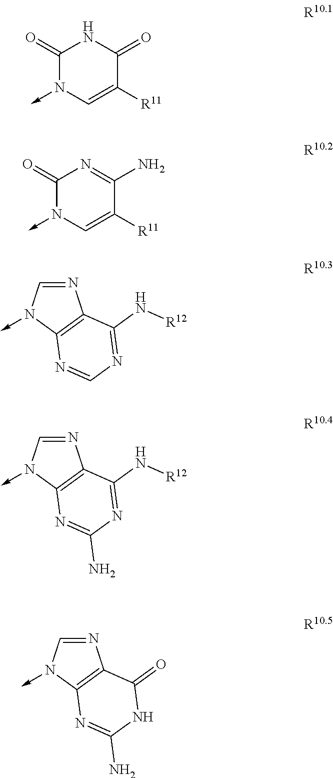
an arrow (→) indicates the place of substituent connection,
R¹¹ is the substituent selected from H, F, Cl, CH₃, or CF₃;
R¹² is hydrogen, C₁-C₄-alkyl or C₃-C₆-cycloalkyl;
X is oxygen or ethanediyl-1,1 (C═CH₂);
Y is O, S, CH₂, or HO—CH group provided when continuous line and its accompanying dotted line ( [symbol] ) together are the single carbon-carbon (C—C) bond,
or
Y is CH group provided when continuous line and its accompanying dotted line ( [symbol] ) together are the double carbon-carbon bond (C═C), and compound of the general formula 1, stereoisomers, isotope-enriched analogues, pharmaceutically acceptable salts, hydrates, solvates, or crystalline or polymorphic forms thereof, wherein:
Ar is aryl or hetaryl;
R¹ is H or CH₃;
R² is isopropyl;
Nuc is 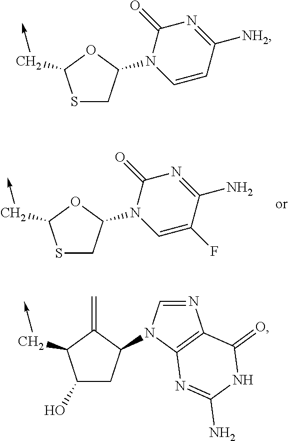
(S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)),
(S)-1-cyclopropoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(4)),
benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), 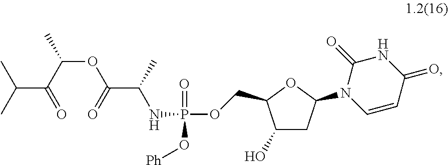 [SMILES: CC(C)C(=O)[C@H](C)OC(=O)[C@H](C)N[P@](=O)(OC[C@H]1O[C@@H](n2ccc(=O)[nH]c2=O)C[C@@H]1O)Oc1ccccc1]
(S)-1-isopropoxycarbonyl-ethyl (S)-2-{(S)-[(1S,4R)-4(2-amino-6-cyclopropylamino-9H-purin-9-yl)-cyclopent-2-enylmethoxy]phenoxy-phosphorylamino}-propanoate (1.2(76)),
allyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(77)),
prop-2-ynyl (S)-2-{(S)-[(1S,4R)-4-(2-amino-6-cyclopropylamino-9H-purin-9-yl)-cyclopent-2-enylmethoxy]-phenoxy-phosphorylamino}propanoate (1.2(78)),
2-methoxy-ethyl (S)-2-{(S)-[(1S,4R)-4-(2-amino-6-cyclopropylamino-9H-purin-9-yl)-cyclopent-2-enylmethoxy]-phenoxy-phosphorylamino}propanoate (1.2(79)),
isopropyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1(1)),
isopropyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1(2)),
isopropyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1(3)),
with the exception of following compounds:

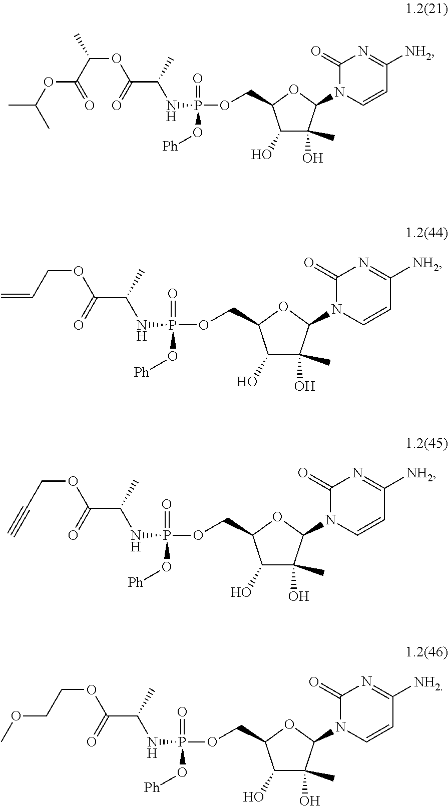 [SMILES: CC(C)OC(=O)[C@H](C)OC(=O)[C@H](C)N[P@](=O)(OC[C@H]1O[C@@H](n2ccc(N)nc2=O)[C@](C)(O)[C@@H]1O)Oc1ccccc1 ; C=CCOC(=O)[C@H](C)N[P@](=O)(OC[C@H]1O[C@@H](n2ccc(N)nc2=O)[C@](C)(O)[C@@H]1O)Oc1ccccc1 ; C#CCOC(=O)[C@H](C)N[P@](=O)(OC[C@H]1O[C@@H](n2ccc(N)nc2=O)[C@](C)(O)[C@@H]1O)Oc1ccccc1 ; COCCOC(=O)[C@H](C)N[P@](=O)(OC[C@H]1O[C@@H](n2ccc(N)nc2=O)[C@](C)(O)[C@@H]1O)Oc1ccccc1]

2. The phosphoramidate nucleoside prodrug of claim 1 is a compound of formula 1.1 and 1.2, a stereoisomer, isotope-enriched analogue, pharmaceutically acceptable salt, hydrate, solvate, or crystalline or polymorphic form thereof, 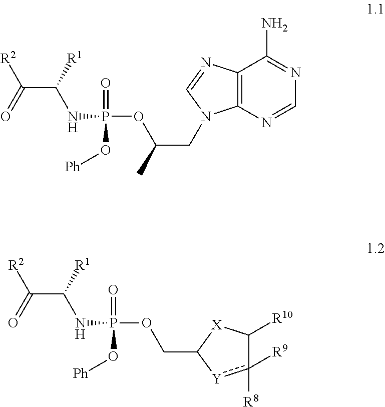
wherein: R¹, R², R⁸, R⁹, R¹⁰, X, Y, and continuous line and its accompanying dotted line ( [symbol] ) have the meaning, defined in claim 1.

3. The phosphoramidate nucleoside prodrug of claim 1 selected from the group consisting of: allyl (S)-2-{(S)-[(R)-1-methyl-2-(6-amino-9H-purin-9-yl)-ethoxy]-phenoxy-phosphorylamino}-propanoate (1.1(1)), 2-methoxy-ethyl (S)-2-{(S)-[(R)-1-methyl-2-(6-amino-9H-purin-9-yl)-ethoxy]-phenoxy-phosphorylamino}-propanoate (1.1(2)), (S)-1-ethoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(1)), (S)-1-isopropyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(2)), (S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)), (S)-1-cyclopropoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(4)), isopropyl (S)-2-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionylamino)-propanoate (1.2(5)), (S)-1-carboxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionate (1.2(6)), (S)-1-carbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(7)), (S)-1-dimethylcarbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(8)), allyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(9)), prop-2-ynyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(10)), 2-methoxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(11)), (S)-1-isopropoxycarbonyl-ethyl (S)-2-{(R)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(12)), (S)-1-isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13)), benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), 4-((S)-2-{[(2R,3R,4R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butyric acid (1.2(15)), (S)-1-isopropoxycarbonyl-ethyl (S)-2-{(S)-[(2R,3S,5R)-5-(2,4-dioxo-5-methyl-3,4-dihydro-2H-pyrimidin-1-yl)-3-hydroxy-tetrahydro-furan-2-ylmethoxy]-phenoxy-phosphorylamino}-propanoate (1.2(17)), (S)-1-isopropoxycarbonyl-ethyl (S)-2-{(S)-[(2R,3S,5R)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(18)), (S)-1-isopropoxycarbonyl-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(19)), (S)-1-isopropoxycarbonyl-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(20)), (S)-1-isopropoxycarbonyl-ethyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(22)), allyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(23)), prop-2-ynyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(24)), 2-methoxy-ethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(25)), allyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(26)), (S)-1-isopropoxycarbonyl-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(27)), (S)-1-isopropoxycarbonyl-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(28)), (S)-1-isopropoxycarbonyl-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(29)), prop-2-ynyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(30)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(31)), prop-2-ynyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(32), 2-methoxy-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(33), allyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(34), allyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(35), prop-2-ynyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(36)), 2-methoxy-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(37)), allyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(38)), prop-2-ynyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(39)), 2-methoxy-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(40)), allyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(41)), prop-2-ynyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(42)), 2-methoxy-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(43)), allyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(47)), prop-2-ynyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(48)), 2-methoxy-ethyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(49)), (S)-1-isopropoxycarbonyl-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(50)), allyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(51)), prop-2-ynyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(52)), 2-methoxy-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(53)), (S)-1-isopropoxycarbonyl-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(54)), (S)-1-isopropoxycarbonyl-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(55)), (S)-1-isopropoxycarbonyl-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(56)), (S)-1-isopropoxycarbonyl-ethyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(57)), allyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(58)), prop-2-ynyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(59)), 2-methoxy-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(60)), allyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (11.2(61))), prop-2-ynyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(62)), 2-methoxy-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(63)), allyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(64)), prop-2-ynyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(65)), 2-methoxy-ethyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(66)), allyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(67)), prop-2-ynyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(68)), 2-methoxy-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(69)), allyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(70)), prop-2-ynyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(71)), 2-methoxy-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(72)), allyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(73)), prop-2-ynyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(74)), 2-methoxy-ethyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(75)), (S)-1-isopropoxycarbonyl-ethyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(76)), allyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(77)), prop-2-ynyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(78)), 2-methoxy-ethyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(79)), (S)-1-isopropoxycarbonyl-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(80)), (S)-1-isopropoxycarbonyl-ethyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(81)), (S)-1-isopropoxycarbonyl-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(82)), allyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(83)), prop-2-ynyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(84)), 2-methoxy-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(85)), allyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(86)), prop-2-ynyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(87)), 2-methoxy-ethyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(88)), allyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(89)), prop-2-ynyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(90)), 2-methoxy-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(91)), (S)-1-isopropoxycarbonyl-ethyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1.2(92)), (S)-1-isopropoxycarbonyl-ethyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1.2(93)), allyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(94)), prop-2-ynyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(95)), 2-methoxy-ethyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(96)), allyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(97)), prop-2-ynyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(98)), 2-methoxy-ethyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(99)), isopropyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1(1)), isopropyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1(2)), isopropyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1(3)), their stereoisomers, isotope-enriched analogues, pharmaceutically acceptable salts, hydrates, solvates, or crystalline or polymorphic forms thereof.

4. A composition comprising the compound of claims 1-3 or its stereoisomer, isotope-enriched analogue, pharmaceutically acceptable salt, hydrate, solvate, or crystalline or polymorphic form thereof and a pharmaceutically acceptable medium.

5. The composition for treating viral and cancerous diseases, which comprises an effective amount of the compound of claims 1-3, or its stereoisomer, isotope-enriched analogue, pharmaceutically acceptable salt, hydrate, solvate, or crystalline or polymorphic form thereof.

6. The composition for treating a hepatitis C virus, which comprises an effective amount of the compound of claims 1-3 or its, stereoisomer, isotope-enriched analogue, pharmaceutically acceptable salt, hydrate, solvate, or crystalline or polymorphic form thereof and a pharmaceutically acceptable medium.

7. The composition for treating a hepatitis C virus, which comprises an effective amount of the compound of claims 1-3 or its stereoisomer, isotope-enriched analogue, pharmaceutically acceptable salt, hydrate, solvate, or crystalline or polymorphic form thereof and effective amount of the NS5A inhibitor and a pharmaceutically acceptable medium.

8. The composition for treating a hepatitis C virus, which comprises an effective amount of the compound of claims 1-3 or its stereoisomer, isotope-enriched analogue, pharmaceutically acceptable salt, hydrate, solvate, or crystalline or polymorphic form thereof, and effective amount of AV-4056 or AV-4058, or AV-4067 and a pharmaceutically acceptable medium.

9. The composition for treating a hepatitis B virus, which comprises an effective amount of the compound of claims 1-3 or its stereoisomer, isotope-enriched analogue, pharmaceutically acceptable salt, hydrate, solvate, or crystalline or polymorphic form thereof and a pharmaceutically acceptable medium.

10. The composition for treating a human immunodeficiency virus, which comprises an effective amount of the compound of claims 1-3 or its stereoisomer, isotope-enriched analogue, pharmaceutically acceptable salt, hydrate, solvate, or crystalline or polymorphic form thereof and a pharmaceutically acceptable medium.

11. The composition comprising the compound as claimed in claim 3 or its stereoisomer, isotope-enriched analogue, pharmaceutically acceptable salt, hydrate, solvate, or crystalline or polymorphic form thereof.

12. A method of treatment viral and cancerous diseases in a subject in need thereof, said method comprises alternatively or concurrently administering a therapeutically effective of a compound of claims 1-3 and another antiviral or anticancer agent to the subject.

13. The method of treatment a hepatitis C virus infection in a subject in need thereof, which comprises administering to the subject an effective amount of the compound of claims 1-3 or a stereoisomer, a hydrate, a solvate, a crystalline form thereof.

14. The method of treatment a hepatitis B virus infection in a subject in need thereof, which comprises administering to the subject an effective amount of the compound of claims 1-3 or a stereoisomer, a hydrate, a solvate, a crystalline form thereof.

15. The method of treatment a human immunodeficiency virus infection in a subject in need thereof, which comprises administering to the subject an effective amount of the of the compound of claims 1-3 or a stereoisomer, a hydrate, a solvate, a crystalline form thereof.

16. The method of treatment a hepatitis C virus and a hepatitis B virus infection in a subject in need thereof, which comprises administering to the subject an effective amount of the compound of claims 1-3 or a stereoisomer, a hydrate, a solvate, a crystalline form thereof.

17. The method of treating a hepatitis C virus and a human immunodeficiency virus infection in a subject in need thereof, which comprises administering to the subject an effective amount of the compound of claims 1-3 or a stereoisomer, a hydrate, a solvate, a crystalline form thereof.

18. The method of treating a hepatitis B virus and a human immunodeficiency virus infection in a subject in need thereof, which comprises administering to the subject an effective amount of the compound of claims 1-3 or a stereoisomer, a hydrate, a solvate, a crystalline form thereof.

19. The method of treating a hepatitis C virus, a hepatitis B virus and a human immunodeficiency virus infection in a subject in need thereof, which comprises administering to the subject an effective amount of the of the compound of claims 1-3 or a stereoisomer, a hydrate, a solvate, a crystalline form thereof.

20. The method of treating according to claim 12, wherein the compound is selected from: allyl (S)-2-{(S)-[(R)-1-methyl-2-(6-amino-9H-purin-9-yl)-ethoxy]-phenoxy-phosphorylamino}-propanoate (1.1(1)), 2-methoxy-ethyl (S)-2-{(S)-[(R)-1-methyl-2-(6-amino-9H-purin-9-yl)-ethoxy]-phenoxy-phosphorylamino}-propanoate (1.1(2)), (S)-1-ethoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(1)), (S)-1-isopropyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(2)), (S)-1-benzyloxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(3)), (S)-1-cyclopropoxycarbonyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(4)), isopropyl (S)-2-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionylamino)-propanoate (1.2(5)), (S)-1-carboxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionate (1.2(6)), (S)-1-carbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(7)), (S)-1-dimethylcarbamoyl-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(8)), allyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(9)), prop-2-ynyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(10)), 2-methoxy-ethyl (S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(11)), (S)-1-isopropoxycarbonyl-ethyl (S)-2-{(R)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(12)), (S)-1-isopropoxycarbonyl-ethyl (S)-2-{[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(13)), benzyl 4-((S)-2-{[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butanoate (1.2(14)), 4-((S)-2-{[(2R,3R,4R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propionyloxy)-butyric acid (1.2(15)), (S)-1-isopropoxycarbonyl-ethyl (S)-2-{(S)-[(2R,3S,5R)-5-(2,4-dioxo-5-methyl-3,4-dihydro-2H-pyrimidin-1-yl)-3-hydroxy-tetrahydro-furan-2-ylmethoxy]-phenoxy-phosphorylamino}-propanoate (1.2(17)), (S)-1-isopropoxycarbonyl-ethyl (S)-2-{(S)-[(2R,3S,5R)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(18)), (S)-1-isopropoxycarbonyl-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(19)), (S)-1-isopropoxycarbonyl-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(20)), (S)-1-isopropoxycarbonyl-ethyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(22)), allyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(23)), prop-2-ynyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(24)), 2-methoxy-ethyl (S)-2-{(S)-[(2R,3S,5R)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(25)), allyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(26)), (S)-1-isopropoxycarbonyl-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(27)), (S)-1-isopropoxycarbonyl-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(28)), (S)-1-isopropoxycarbonyl-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(29)), prop-2-ynyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(30)), 2-methoxy-ethyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(31)), prop-2-ynyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(32), 2-methoxy-ethyl (S)-2-{(S)-[(2R,3R,4R,5R)-5-(2,4-dioxo-3,4-dihydro-2H-pyrimidin-1-yl)-4-fluoro-3-hydroxy-4-methyl-tetrahydro-furan-2-ylmethoxy]-phenoxy-phosphorylamino}-propanoate (1.2(33), allyl (S)-2-{(S)-[(2R,3R,5R)-5-(2,4-dioxo-3,4-dihydro-2H-pyrimidin-1-yl)-4,4-difluoro-3-hydroxy-tetrahydro-furan-2-ylmethoxy]-phenoxy-phosphorylamino}-propanoate (1.2(34), allyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(35), prop-2-ynyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(36)), 2-methoxy-ethyl (S)-2-{(S)-[(2R,3R,4R,5R)-5-(4-amino-2-oxo-2H-pyrimidin-1-yl)-4-fluoro-3-hydroxy-4-methyl-tetrahydro-furan-2-ylmethoxy]-phenoxy-phosphorylamino}-propanoate (1.2(37)), allyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(38)), prop-2-ynyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(39)), 2-methoxy-ethyl (S)-2-{(S)-[(2R,3R,5R)-5-(4-amino-2-oxo-2H-pyrimidin-1-yl)-4,4-difluoro-3-hydroxy-tetrahydro-furan-2-ylmethoxy]-phenoxy-phosphorylamino}-propanoate (1.2(40)), allyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(41)), prop-2-ynyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(42)), 2-methoxy-ethyl (S)-2-{(S)-[(2R,3R,4S,5R)-5-(4-amino-2-oxo-2H-pyrimidin-1-yl)-4-chloro-4-fluoro-3-hydroxy-tetrahydro-furan-2-ylmethoxy]-phenoxy-phosphorylamino}-propanoate (1.2(43)), allyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(47)), prop-2-ynyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(48)), 2-methoxy-ethyl (S)-2-{(S)-[(2R,3S,5R)-5-(6-amino-9H-purin-9-yl)-3-hydroxy-tetrahydro-furan-2-ylmethoxy]-phenoxy-phosphorylamino}-propanoate (1.2(49)), (S)-1-isopropoxycarbonyl-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(50)), allyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(51)), prop-2-ynyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(52)), 2-methoxy-ethyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1.2(53)), (S)-1-isopropoxycarbonyl-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(54)), (S)-1-isopropoxycarbonyl-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(55)), (S)-1-isopropoxycarbonyl-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(56)), (S)-1-isopropoxycarbonyl-ethyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(57)), allyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(58)), prop-2-ynyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(59)), 2-methoxy-ethyl (S)-2-{(S)-[(2S,3R,5S)-5-(2,4-dioxo-3,4-dihydro-2H-pyrimidin-1-yl)-3-hydroxy-tetrahydro-furan-2-ylmethoxy]-phenoxy-phosphorylamino}-propanoate (1.2(60)), allyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (11.2(61))), prop-2-ynyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(62)), 2-methoxy-ethyl (S)-2-{(S)-[(2S,3R,5S)-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(63)), allyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(64)), prop-2-ynyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(65)), 2-methoxy-ethyl (S)-2-{(S)-[(2S,3R,5S)-5-(2,4-dioxo-5-trifluoromethyl-3,4-dihydro-2H-pyrimidin-1-yl)-3-hydroxy-tetrahydro-furan-2-ylmethoxy]-phenoxy-phosphorylamino}-propanoate (1.2(66)), allyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(67)), prop-2-ynyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(68)), 2-methoxy-ethyl (S)-2-{(S)-[(2S,3S,4R,5S)-4-fluoro-3-hydroxy-5-(5-methyl-2,4-dioxo-3,4-dihydro-2H-pyrimidin-1-yl)-tetrahydro-furan-2-ylmethoxy]-phenoxy-phosphorylamino}-propanoate (1.2(69)), allyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(70)), prop-2-ynyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(71)), 2-methoxy-ethyl (S)-2-{(S)-[(2S,3R,5S)-5-(4-amino-2-oxo-2H-pyrimidin-1-yl)-3-hydroxy-tetrahydro-furan-2-ylmethoxy]-phenoxy-phosphorylamino}-propanoate (1.2(72)), allyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(73)), prop-2-ynyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(74)), 2-methoxy-ethyl (S)-2-{(S)-[(2S,3R,5S)-5-(6-amino-9H-purin-9-yl)-3-hydroxy-tetrahydro-furan-2-ylmethoxy]-phenoxy-phosphorylamino}-propanoate (1.2(75)), (S)-1-isopropoxycarbonyl-ethyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(76)), allyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(77)), prop-2-ynyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(78)), 2-methoxy-ethyl (S)-2-{(S)-[(1S,4R)-4-(2-amino-6-cyclopropylamino-9H-purin-9-yl)-cyclopent-2-enylmethoxy]-phenoxy-phosphorylamino}-propanoate (1.2(79)), (S)-1-isopropoxycarbonyl-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(80)), (S)-1-isopropoxycarbonyl-ethyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(81)), (S)-1-isopropoxycarbonyl-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(82)), allyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(83)), prop-2-ynyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(84)), 2-methoxy-ethyl (S)-2-{(S)-[(2R,4R)-4-(2,4-dioxo-3,4-dihydro-2H-pyrimidin-1-yl)-[1,3]dioxolan-2-ylmethoxy]-phenoxy-phosphorylamino}-propanoate (1.2(85)), allyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(86)), prop-2-ynyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(87)), 2-methoxy-ethyl (S)-2-{(S)-[(2R,4R)-4-(6-cyclopropylamino-9H-purin-9-yl)-[1,3]dioxolan-2-ylmethoxy]-phenoxy-phosphorylamino}-propanoate (1.2(88)), allyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(89)), prop-2-ynyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(90)), 2-methoxy-ethyl (S)-2-{(S)-[(2R,4R)-4-(2-amino-6-oxo-1,6-dihydro-purin-9-yl)-[1,3]dioxolan-2-ylmethoxy]-phenoxy-phosphorylamino}-propanoate (1.2(91)), (S)-1-isopropoxycarbonyl-ethyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1.2(92)), (S)-1-isopropoxycarbonyl-ethyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1.2(93)), allyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(94)), prop-2-ynyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(95)), 2-methoxy-ethyl (S)-2-{(S)-[(2R,5S)-5-(2,4-dioxo-3,4-dihydro-2H-pyrimidin-1-yl)-[1,3]oxathiolan-2-ylmethoxy]-phenoxy-phosphorylamino}-propanoate (1.2(96)), allyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(97)), prop-2-ynyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(98)), 2-methoxy-ethyl (S)-2-{(S)-[(2R,5S)-5-(5-fluoro-2,4-dioxo-3,4-dihydro-2H-pyrimidin-1-yl)-[1,3]oxathiolan-2-ylmethoxy]-phenoxy-phosphorylamino}-propanoate (1.2(99)), isopropyl (S)-2-{(S)-[(2R,5S)-5-(4-amino-2-oxo-2H-pyrimidin-1-yl)-[1,3]oxathiolan-2-ylmethoxy]-phenoxy-phosphorylamino}-propanoate (1(1)), isopropyl (S)-2-{(S)-[(2R,5S)-5-(4-amino-5-fluoro-2-oxo-2H-pyrimidin-1-yl)-[1,3]oxathiolan-2-ylmethoxy]-phenoxy-phosphorylamino}-propanoate (1(2)), isopropyl (S)-2-{(S)-[(1R,3S,5S)-3-(2-amino-6-oxo-1,6-dihydro-purin-9-yl)-5-hydroxy-2-methylene-cyclopentylmethoxy]-phenoxy-phosphorylamino}-propanoate (1(3)), their stereoisomers, isotope-enriched analogues, pharmaceutically acceptable salts, hydrates, solvates, or crystalline or polymorphic forms thereof.

21. A process for preparing the phosphoramidate nucleoside prodrug of claims 1-3, or its stereoisomers, isotope-enriched analogues, pharmaceutically acceptable salts, hydrates, solvates, crystalline or polymorphic forms thereof, comprising the procedure for the synthesis of phosphoramidate nucleoside prodrugs where a compound of formula 2 or a stereoisomer thereof undergoes a reaction with substituted alcohol of formula 3 or stereoisomer thereof 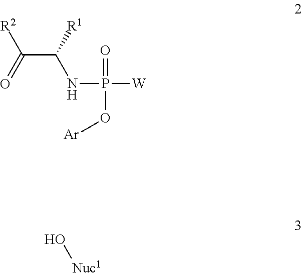
wherein: Ar, R¹ and R² have the meaning, according to claim 1; W is Cl or pentafluorophenyloxy; Nuc¹ is Nuc which has the above meaning and not necessarily having an O-protecting group or/and a N-protecting group.

22. The process according to claim 21 for preparing a nucleoside prodrug phosphoric acid of claims 1-3 or a stereoisomer, isotopically enriched analog, pharmaceutically acceptable salt, hydrate, solvate or crystalline or polymorphic forms thereof, wherein the reagent of formula 2 is a compound of formulas 2(1)-2(14) 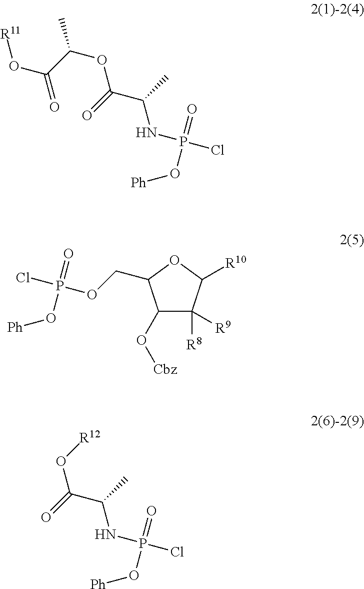
wherein: 2(1): R¹¹=Et. 2(2): R¹¹=i-Pr. 2(3): R¹¹═CH₂Ph. 2(4): R¹¹=c-Pr. 2(5): Cbz=protected group, X has the meaning, according to claim 1, 2(6): R¹²═CH₂—CH═CH₂. 2(7): R¹²═CH₂—C≡CH. 2(8): R¹²═CH₂CH₂OCH₃. 2(9): R¹²═CH₂CH₂CH₂CO₂CH₂Ph. 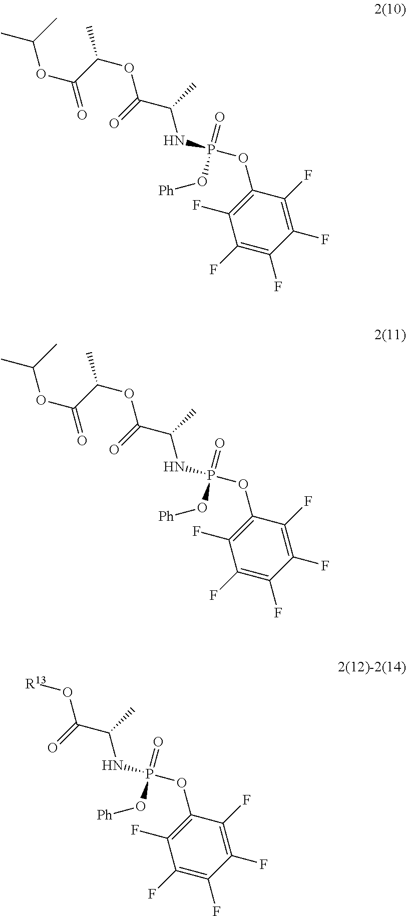
2(12): R¹³=allyl; 2(13): R¹³=prop-2-ynyl; 2(14): R¹³=2-methoxyethyl.
